(19) United States
(12) Patent Application Publication
(10) Pub. No.: US 2026/0014259 A1
(43) Pub. Date: Jan. 15, 2026

(54) PEG TARGETING COMPOUNDS FOR DELIVERY OF THERAPEUTICS

(71) Applicant: ModernaTX, Inc., Cambridge, MA (US)

(72) Inventors: Mohindra SEEPERSAUD, Acton, MA (US); Junyong KIM, Cambridge, MA (US); Sathyajith Ellalahewage KUMARASINGHE, Acton, MA (US); Sreyoshi SUR, Rochester, NY (US)

(73) Assignee: ModernaTX, Inc., Cambridge, MA (US)

(21) Appl. No.: 19/335,869

(22) Filed: Sep. 22, 2025

Related U.S. Application Data
(63) Continuation of application No. PCT/US2024/021352, filed on Mar. 25, 2024.
(60) Provisional application No. 63/454,253, filed on Mar. 23, 2023.

(51) Int. Cl. A61K 47/54; A61K 47/60; A61K 47/69; C12N 9/22

(52) U.S. Cl. CPC A61K 47/544; A61K 47/60; A61K 47/6911; C12N 9/222

(57) ABSTRACT

Provided herein are targeting compounds (e.g., a compound of Formula I, a stereoisomer thereof, a tautomer thereof, and/or a pharmaceutically acceptable salt thereof), lipid nanoparticle (LNP) compositions comprising such targeting compounds and the use thereof. The LNP compositions described herein may further comprise one or more selected from ionizable lipids, PEG-lipids, phospholipids, and structural lipids.

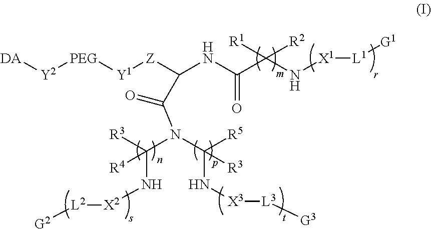

### CROSS-REFERENCE TO RELATED APPLICATIONS

This application is a continuation of International PCT Application No. PCT/US2024/021352 filed Mar. 25, 2024, which application claims the benefit of priority to U.S. Provisional Application No. 63/454,253, filed Mar. 23, 2023, the entire contents of both of which are incorporated herein by reference.

### TECHNICAL FIELD

Provided herein are PEG targeting compounds for use in targeted lipid assemblies (TLAs) and processes for their preparation, as well as compositions and use of TLAs.

### BACKGROUND

The following description of the background of the present technology is provided simply as an aid in understanding the present technology and is not admitted to describe or constitute prior art to the present technology.

The use of lipid nanoparticle (LNP) compositions comprising payload molecules, such as nucleic acid molecules, for the effective delivery of such molecules to target tissues represents a continuing medical challenge. In particular, the delivery of nucleic acid molecules to target cells is complicated by the relative instability and low cell permeability of such molecules. Accordingly, there is a need to develop targeting compounds and compositions thereof to facilitate the delivery of therapeutic or prophylactic payload molecules, such as nucleic acid molecules, to the target cells.

### SUMMARY

The present disclosure provides, among other things, targeting compounds (e.g., a compound of Formula I) and processes for their preparation, as well as targeted lipid assembly (TLA) compositions (e.g., targeted lipid nanoparticle (TLNP) compositions) comprising such targeting compounds and the use thereof. Thus. in one aspect, the present technology provides targeting compounds of Formula I, a stereoisomer thereof, a tautomer thereof, and/or a pharmaceutically acceptable salt thereof:

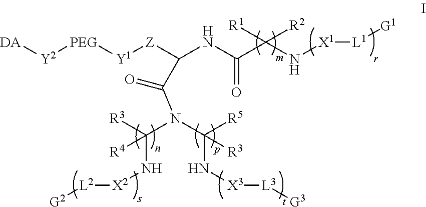

wherein,
DA is di(C₁₂₋₂₄ alkanoyl)glycero or di(C₁₂₋₂₄ alkyl)glycero;
G¹, G², and G³ are each independently selected from an asialoglycoprotein receptor targeting monosaccharide;
L¹, L², and L³ are, at each occurrence, independently selected from alkylene, alkenylene, heteroalkylene, cycloalkylene, heterocyclylene, arylene, or heteroarylene groups, or any combination of 2 or 3 of the foregoing groups;
PEG is a poly(ethylene glycol) having 1 to 100 ethylene oxy subunits;
X¹, X² and X³ are, at each occurrence, independently absent, C(O), C(O)O, or C(O)NH;
Y¹ is absent or a C₁₋₆ alkylene, O, C₁₋₆ alkylene-O, NH, C₁₋₆ alkylene-NH, C(O), C(O)NH, or C₁₋₆ alkylene-C(O)NH group;
Y² is absent or an O, C₁₋₆ alkylene-O, NH, C₁₋₆ alkylene-NH, C(O), C(O)O, or C(O)NH group;
Z is C₁₋₆ alkylene or

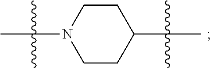 [SMILES: *C1CCN(*)CC1]

R¹, R², R³, R⁴, R⁵, and R⁶ at each occurrence are independently H or a C₁₋₆ alkyl group;
m is 1, 2, 3, 4, 5, or 6;
n, and p are each independently selected from 2, 3, 4, 5 or 6; and
r, s, and t are each independently 1, 2, 3, 4, or 5.

In another aspect, the present technology provides targeted lipid assembly (TLA) and pharmaceutically acceptable salts thereof, wherein the TLA comprises: a targeting compound as described herein; an ionizable lipid; a structural lipid; a phospholipid; and a PEG-lipid.

In another aspect, the technology provides methods of specifically delivering a therapeutic and/or prophylactic agent to a target cell within a subject comprising administering any TLA as described herein to the subject. The target cell may be a hepatocyte.

In still another aspect, there are provided method of producing a polypeptide of interest in a target cell within a subject, comprising administering any TLA as described herein to the subject. The target cell may be a hepatocyte.

In yet another aspect, the present technology provides methods of editing a gene in a target cell within a subject comprising administering any TLA including a gene editing system as described herein to the subject. The target cell may be a hepatocyte.

### DETAILED DESCRIPTION

The present disclosure provides, inter alia, targeting compounds (e.g., a compound of Formula I) and processes for their preparation, as well as targeted lipid assembly (TLA) compositions, including targeted lipid nanoparticle (TLNP) compositions comprising such targeting compounds and the use thereof. In some embodiments, the targeting compounds described herein are useful as liposomal compositions or as components of liposomal compositions to facilitate the delivery to, and/or subsequent transfection of one or more target cells. In some embodiments, the TLA/TLNP compositions described herein further comprise one or more selected from ionizable lipids, PEG-lipids, phospholipids, and structural lipids.

### Targeting Compounds

Targeting compounds disclosed herein comprise a targeting moiety (e.g., an asialoglycoprotein receptor targeting monosaccharide). In embodiments, the targeting compounds described herein can provide one or more advantageous characteristics or properties. That is, in certain embodiments, a targeted lipid assembly (TLA) comprising the targeting compound described herein (e.g., a compound of Formula I) can be characterized as having one or more properties that afford such TLA advantages relative to delivery systems (e.g., liposomal-based vehicles) without such targeting compounds. For example, the targeting compound described herein can allow enhanced editing percentage in a CRISPR/Cas9 technology.

In one aspect, provided herein is a targeting compound having Formula I, a stereoisomer thereof, a tautomer thereof, and/or a pharmaceutically acceptable salt thereof:

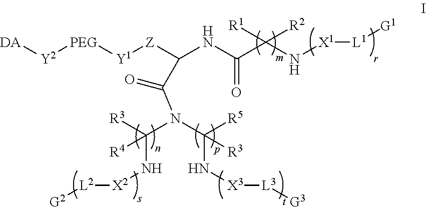

wherein,
DA is di(C₁₂₋₂₄ alkanoyl)glycero or di(C₁₂₋₂₄ alkyl)glycero;
G¹, G², and G³ are each independently selected from an asialoglycoprotein receptor targeting monosaccharide;
L¹, L², and L³ are, at each occurrence, independently selected from alkylene, alkenylene, heteroalkylene, cycloalkylene, heterocyclylene, arylene, or heteroarylene groups, or any combination of 2 or 3 of the foregoing groups;
PEG is a poly(ethylene glycol) having 1 to 100 ethylene oxy subunits;
X¹, X² and X³ are, at each occurrence, independently absent, C(O), C(O)O, or C(O)NH;
Y¹ is absent or a C₁₋₆ alkylene, O, C₁₋₆ alkylene-O, NH, C₁₋₆ alkylene-NH, C(O), C(O)NH, or C₁₋₆ alkylene-C(O)NH group;
Y² is absent or an O, C₁₋₆ alkylene-O, NH, C₁₋₆ alkylene-NH, C(O), C(O)O, or C(O)NH group;
Z is C₁₋₆ alkylene or

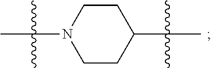 [SMILES: *C1CCN(*)CC1]

R¹, R², R³, R⁴, R⁵, and R⁶ at each occurrence are independently H or a C₁₋₆ alkyl group;
m is 1, 2, 3, 4, 5, or 6;
n, and p are each independently selected from 2, 3, 4, 5 or 6; and
r, s, and t are each independently 1, 2, 3, 4, or 5.

In some embodiments the targeting compound of Formula I has the structure of Formula

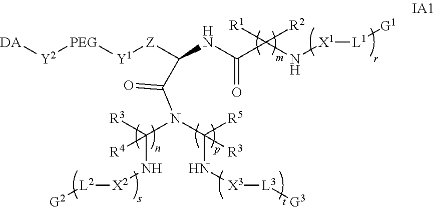

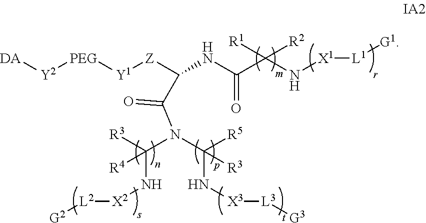

In some embodiments of the targeting compounds herein, Z may be C₁₋₆ alkylene.

In some embodiments, the targeting compound of Formula I has the structure of Formula IB.

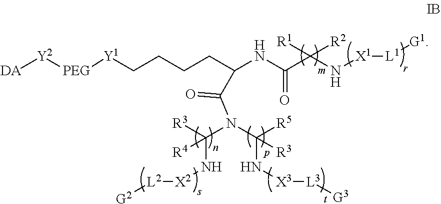

In some embodiments of the targeting compounds herein, Z may be

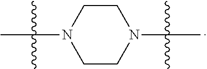 [SMILES: *N1CCN(*)CC1]

In some embodiments, the targeting compound of Formula I has the structure of Formula IC:

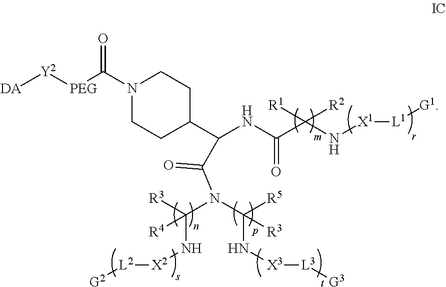

In some embodiments of the targeting compounds herein, X¹ may be C(O) or C(O)O. In some embodiments, X¹ may be C(O).

In some embodiments of the targeting compounds herein, X² may be C(O) or C(O)O. In some embodiments, X² may be C(O).

In some embodiments of the targeting compounds herein, X³ may be C(O) or C(O)O. In some embodiments, X³ may be C(O).

In some embodiments, the targeting compound of Formula I has the structure of Formula ID:

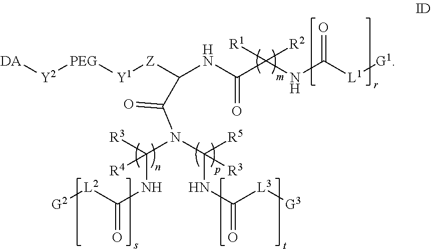

In some embodiments of the targeting compounds herein, r, s, and t may each independently be 1, 2, 3, or 4. In some embodiments, r, s, and t may each independently be 1, 2, or 3. In some embodiments, r may be 1 or 2. In some embodiments, s may be 1 or 2. In some embodiments, t may be 1 or 2. In some embodiments, r, s, and t are each 1. In some embodiments, r, s, and t are each 2.

In some embodiments, the targeting compound of Formula I has the structure of Formula IE:

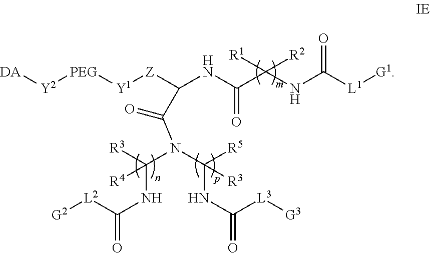

In some embodiments, the targeting compound of Formula I has the structure of Formula IF or IG:

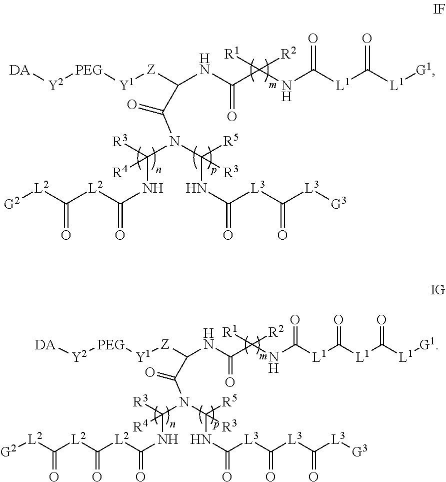

In some embodiments of the targeting compounds herein, m may be selected from 1, 2, 3, 4, 5, or 6, and n and p may be each independently selected from 2, 3, 4, 5 or 6. In some embodiments, m may be 1, 2, 3, or 4. In some embodiments, m may be 2 or 3. In some embodiments, n may be 2, 3, or 4. In some embodiments, n may be 2 or 3. In some embodiments, p may be 2, 3 or 4. In some embodiments, p may be 2 or 3.

In some embodiments of the targeting compounds herein, L¹, L², and L³ are, at each occurrence, independently selected from alkylene, alkenylene, heteroalkylene, cycloalkylene, heterocyclylene, arylene, or heteroarylene groups, or any combination of 2 or 3 of the foregoing groups. In some embodiments, the alkylene, alkenylene, heteroalkylene or cycloalkylene may be optionally substituted with one or more hydroxyl, halogen, benzyl, or oxo (C═O) groups (e.g., 1, 2, or 3 such groups), and the arylene and heteroarylene groups are optionally substituted with 1, 2, 3, or 4 C₁₋₄ alkyl or halogen groups. In some embodiments, L¹, L², and L³ at each occurrence are each independently selected from linear alkylene, linear heteroalkylene, arylene, or heteroarylene groups, or any combination of 2 of the foregoing groups, wherein the alkylene or heteroalkylene are optionally substituted with one or more (e.g., 1, 2, or 3) hydroxyl, halogen, benzyl, or oxo (C═O) groups, and the arylene and heteroarylene groups are optionally substituted with 1, 2, 3, or 4 C₁₋₄ alkyl or halogen groups. In some embodiments, L¹, L², and L³ at each occurrence are each independently selected from a linear alkylene group or a linear heteroalkylene group having 1 to 4 heteroatoms selected from NH, O, or S, wherein the alkylene or heteroalkylene groups are optionally substituted with 1 or 2 oxo (C═O) groups. In some embodiments, L¹, L², and L³ at each occurrence are each independently an unsubstituted linear C₁₋₆ alkylene group or an unsubstituted linear NH—C₁₋₆ alkylene group.

In some embodiments of the targeting compounds herein, L¹, L², and L³ at each occurrence are each independently selected from a linear alkylene group, a linear heteroalkylene group having 1 to 4 heteroatoms selected from NH, O, or S, an arylene group, or a heteroarylene group, wherein the alkylene or heteroalkylene groups are optionally substituted with 1 or 2 oxo (C═O) groups, or a combination of a linear alkylene group and either an arylene group or a heteroarylene group. In some embodiments of the targeting compounds herein, L¹, L², and L³ at each occurrence are each independently an unsubstituted linear C₁₋₆ alkylene group, an unsubstituted linear NH—C₁₋₆ alkylene group, an unsubstituted linear C₁₋₆ heteroalkylene group having an O atom, an unsubstituted aryl-alkylene group, or an unsubstituted heteroaryl-alkylene group. In some embodiments of the targeting compounds herein, L¹, L², and L³ at each occurrence are each independently a C₆₋₁₀ aryl-C₁₋₆ alkylene or a C₁₋₉ heteroaryl-C₁₋₆ alkylene. In some embodiments, the foregoing heteroaryl has 1, 2, 3, or 4 heteroatoms selected from O, N, NH, or S. In some embodiments of the targeting compounds herein, L¹, L², and L³ at each occurrence are each independently a C₁₋₉ heteroaryl-C₁₋₆ alkylene having 1, 2, or 3 N atoms, e.g., C₂₋₅ heteoraryl with 1 or 2 N atoms.

In some embodiments of the targeting compounds herein, L¹, L², and L³ at each occurrence are each independently selected from —(CH₂)—, —(CH₂)₂—, —(CH₂)₃—, —(CH₂)₄—, —CH₂—C(CH₃)₂—(CH₂)₂—, —CH₂—CH(CH₃)—(CH₂)₂—, —NH(CH₂)—, —NH(CH)(CH₃)—, —NH(CH₂)₂—, —NH(CH₂)₃—, —NH(CH₂)₄—, —CH₂—O—CH₂—CH₂—CH₂—, —CH₂—O—CH₂—CH₂—, —CH₂—S—CH₂—CH₂—CH₂—, phenylene, phenylmethylene, pyridinylene, pyridinylmethylene, triazolylene, triazolylmethylene, triazolylethylene, pyrazinylene, pyrazinylmethylene, pyrrolylene, or pyrrolylmethylene. In some embodiments, L¹, L², and L³ at each occurrence are independently selected from —NH(CH₂)—, —NH(CH₂)₂—, —NH(CH₂)₃—, —NH(CH)(CH₃)—, or —NH(CH₂)₄—.

In some embodiments of the targeting compounds herein, R¹, R², R³, R⁴, R⁵, and R⁶ at each occurrence may be independently H or C₁₋₆ alkyl. In some embodiments, the C₁₋₆ alkyl may be optionally substituted with 1 or more halogens, e.g., 1, 2, or 3 F or C₁. In some embodiments, R¹ and R² at each occurrence are H. In some embodiments, R³ and R⁴ at each occurrence are H. In some embodiments, R⁵ and R⁶ at each occurrence are H. In some embodiments, at least one of R¹, R², R³, R⁴, R⁵, and R⁶ is not H. In some embodiments, at least one occurrence of one of R¹, R², R³, R⁴, R⁵, and R⁶ may be a C₁₋₃ alkyl group, e.g., a methyl group. In some embodiments, one occurrence of R¹ or R² may be a methyl group. In some embodiments, one occurrence of R³ or R⁴ may be a methyl group. In some embodiments, one occurrence of R⁵ or R⁶ may be a methyl group. In some embodiments, R¹ and R² are both methyl. In some embodiments, R¹, R², R³, R⁴, R⁵, and R⁶ at each occurrence are H.

In some embodiments of the targeting compounds herein, DA may be di(C₁₂₋₂₄ alkanoyl)glycero or di(C₁₂₋₂₄alkyl)glycerol. In some embodiments, DA may be di(C₁₆₋₂₄ alkanoyl)glycero or di(C₁₆₋₂₄ alkyl)glycero. In some embodiments, DA may be di(C₁₆₋₂₀ alkanoyl)glycero or di(C₁₆₋₂₀ alkyl)glycero. In some embodiments, DA may be distearoylglycero or dioctadecylglycero.

In some embodiments of the targeting compounds herein, Y¹ may be absent, a C₁₋₆ alkylene, O, C₁₋₆ alkylene-O, NH, C₁₋₆ alkylene-NH, C(O), C(O)NH, or C₁₋₆ alkylene-C(O)NH group. In some embodiments, Y¹ may be absent, O, ethyleneoxy, or C₁₋₆ alkylene-C(O)NH group. In some embodiments, Y¹ may be —(CH₂)—C(O)NH.

In some embodiments of the targeting compounds herein, Y² may be absent or an O, C₁₋₆ alkylene-O, NH, C₁₋₆ alkylene-NH, C(O), C(O)O, or C(O)NH group. In some embodiments, Y² may be C(O)NH.

In some embodiments of the targeting compounds herein, G¹, G², and G³ may be each independently selected from an asialoglycoprotein receptor targeting monosaccharide. For example, G¹, G², and G³ each independently may have the structure of Formula A or B:

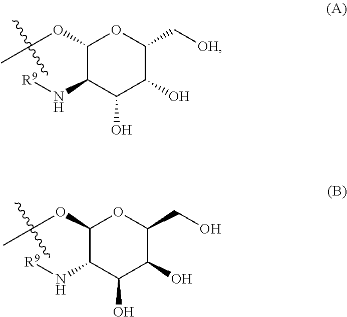

wherein
   R⁹ is R¹⁰C(O), R¹⁰S(O)₂ or R¹⁰OC(O); and
   R¹⁰ is an alkyl or alkenyl group.

In some embodiments, R¹⁰ is a C₁₋₄ alkyl group optionally substituted with 1, 2, or 3 halogens.

In some embodiments of the targeting compounds herein, PEG may be a poly(ethylene glycol) having 1 to 100 ethylene oxy subunits. For example, the PEG may have 5, 10, 15, 20, 25, 30, 35, 40, 45, 50, 55, 60, 65, 70, 75, 80, 85, 90, 95, or 100 ethylene oxy subunits or a range between and including any two of the foregoing values, e.g., 40-50 subunits or about 45 subunits. In some embodiments the PEG has a weight average molecular weight of about 2000. As is known to those of skill in the art, PEG being a polymer, often exhibits polydispersity. Thus, compounds including PEG may be present as polydisperse mixtures. For example, a PEG compound, such as the PEG targeting compounds herein, may be represented as having 45 ethylene oxy subunits but may be present in a mixture also containing PEGs of 44 or 46 ethylene oxy subunits. It will therefore be understood that representations of a PEG compound having a particular number of ethylene oxy subunits, discloses that exact compound and polydisperse mixtures containing that compound.

### Exemplary Targeting Compounds

In some embodiments, a targeting compound is any one described in Table A, a stereoisomer thereof, a tautomer thereof, and/or a pharmaceutically acceptable salt thereof.

TABLE A
Exemplary compounds of Formula I.

| Structure | Compound |
|---|---|
| 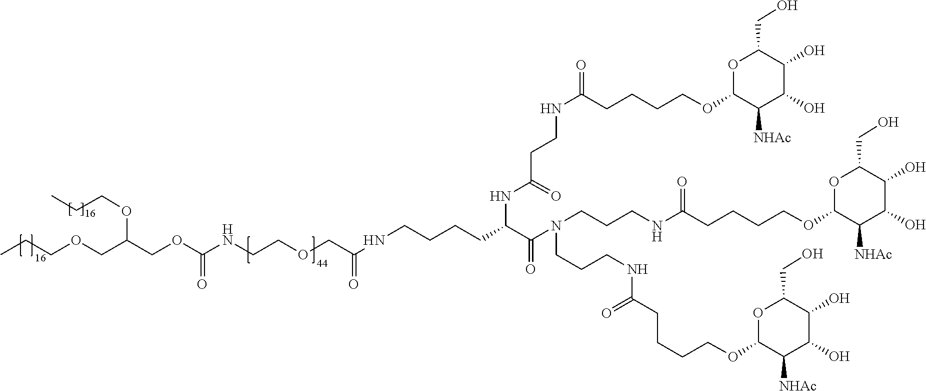 | 14 |
| 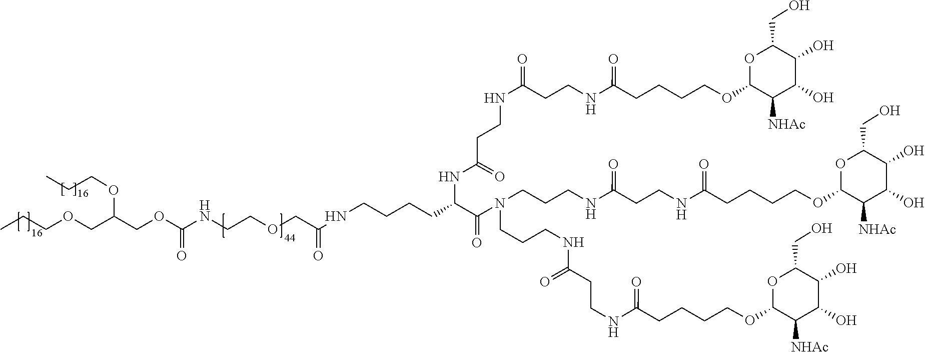 | 17 |
| 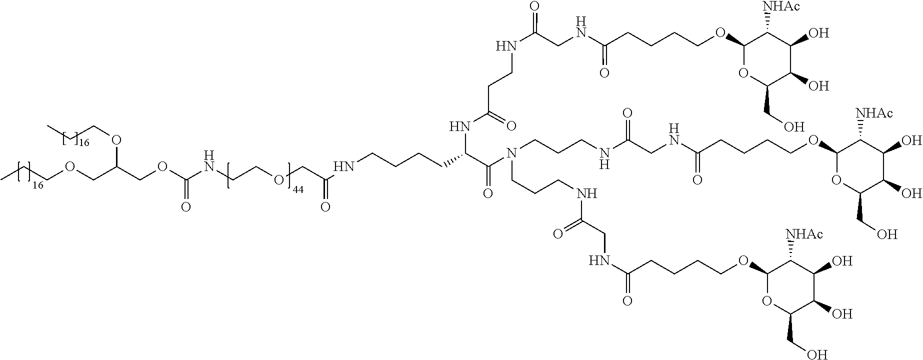 | 20 |
| 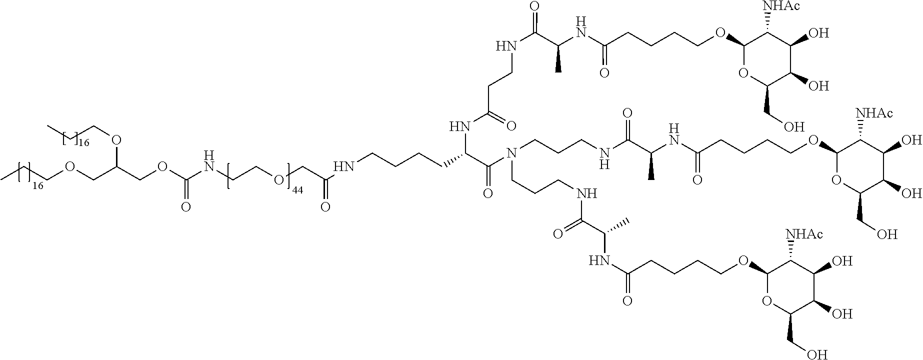 | 23 |
| 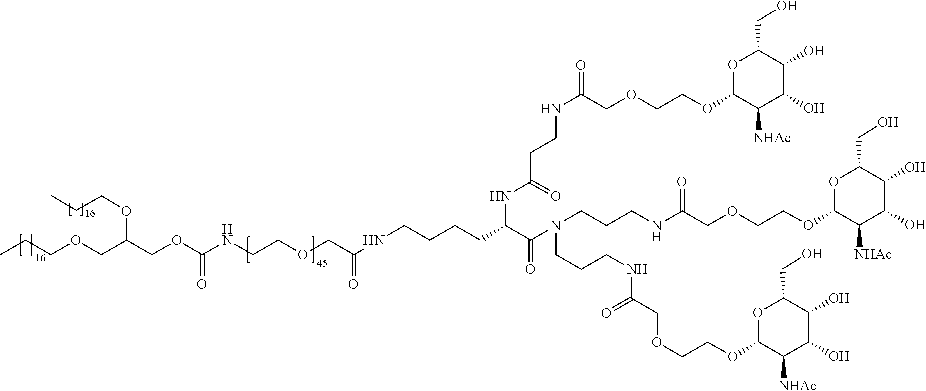 | 38 |
| 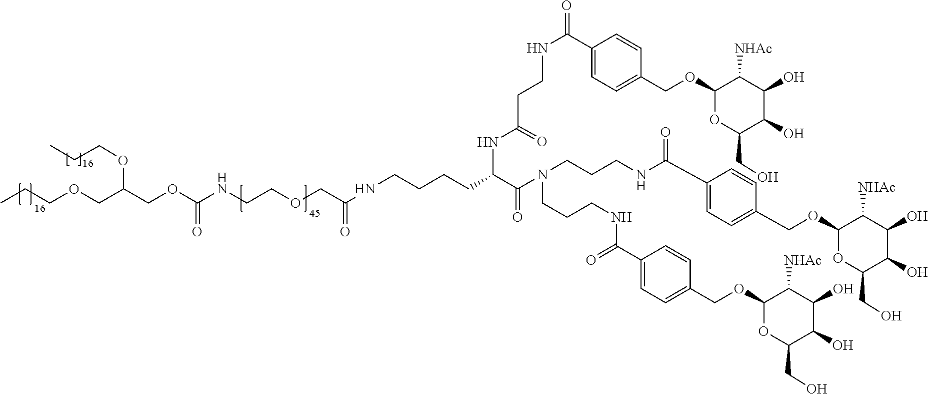 | 39 |
| 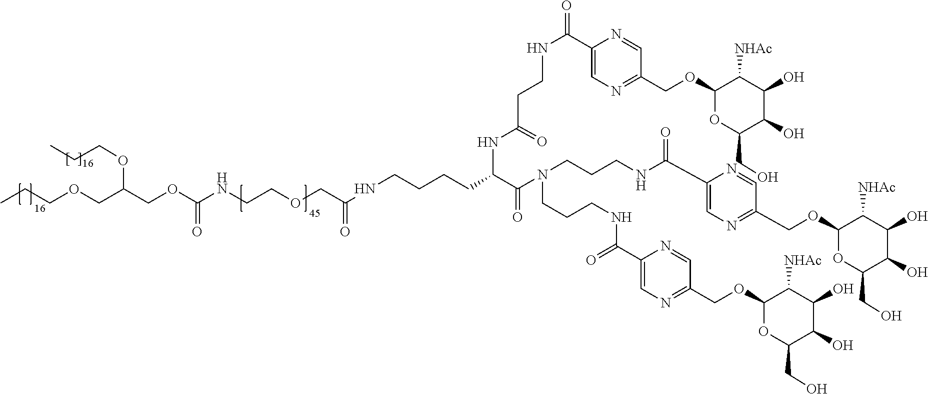 | 40 |

### Targeted Lipid Assembly

Targeting compounds described herein (e.g., a compound of Formula I) may be used in the preparation of targeted lipid assembly (TLA) for delivering payload materials (e.g., gene editing system such as Cas9 and sgRNA) to a target tissue in a subject. In some embodiments, the TLA (e.g., a lipid assembly comprising a targeting compound of Formulas I, IA1, IA2, IB, IC, ID, IE, IF, or IG) further comprises one or more lipids selected from an ionizable lipid, a structural lipid, a phospholipid, and a PEG-lipid. In some embodiments, a TLA comprises a targeting compound such as a compound of Formula I, an ionizable lipid, a structural lipid, a phospholipid (or an alternative lipid), and a PEG-lipid.

In some embodiments, a composition is a suitable delivery vehicle such as a TLA. In embodiments, a composition is a liposomal delivery vehicle, e.g., a lipid nanoparticle.

In some embodiments, a TLA (e.g., a lipid nanoparticle) can have a net positive charge. In embodiments, a TLA (e.g., a lipid nanoparticle) can have a net negative charge. In embodiments, a TLA (e.g., a lipid nanoparticle) can have a net neutral charge.

The amount of a targeting compound as described herein (e.g., a compound of Formula I) in a composition can be described as a percentage (“mol %”) of the combined molar amounts of total lipids of a composition (e.g., the combined molar amounts of all lipids present in a TLA).

In some embodiments, a targeting compound described herein (e.g., a compound of Formula I) is present in an amount that is about 0.025 mol % to about 10 mol % of the combined molar amounts of all lipids present in a composition such as a TLA, including about 0.035 mol %, 0.05 mol %, about 0.1 mol % to about 5 mol %, about 0.1 mol % to about 2 mol %, or about 0.1 mol % to about 1 mol %. In some embodiments, a targeting compound described herein (e.g., a compound of Formula I) is present in an amount that is about 0.1 mol %, about 0.25 mol %, about 0.5 mol %, about 0.75 mol %, about 1 mol %, about 1.5 mol %, about 2 mol %, about 3 mol %, about 4 mol %, or about 5 mol %, or a range between and including any two of the foregoing values, of the combined molar amounts of all lipids present in a composition such as a TLA. In some embodiments, a targeting compound described herein (e.g., a compound of Formula I) is present in an amount that is about 0.1 mol % to about 1 mol % of the combined molar amounts of all lipids present in a composition such as a TLA.

In some embodiments, a TLA further comprises one or more lipids (e.g., one or more lipids selected from the group consisting of one or more ionizable lipids, one or more PEG-lipids, one or more phospholipids (or one or more alternative lipids), and one or more structural lipids).

### Ionizable Lipids

The TLA and lipid nanoparticle compositions of the present technology may include an ionizable lipid (e.g., an ionizable lipid described herein). As used herein, the term “ionizable lipid” has its ordinary meaning in the art and may refer to a lipid comprising one or more charged moieties. In some embodiments, an ionizable lipid may be positively charged or negatively charged. For instance, an ionizable lipid may be positively charged at lower pHs, in which case it could be referred to as “cationic lipid.” In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

In some embodiments, the lipid nanoparticle compositions described herein comprise about 30 mol % to about 65 mol % of ionizable lipid, including about 35 mol % to about 60 mol %, about 40 mol % to about 55 mol %, or about 45 mol % to about 50 mol %. In some embodiments, the nanoparticle described herein comprises about 30 mol %, about 35 mol %, about 40 mol %, about 45 mol %, about 50 mol %, about 55 mol %, about 60 mol %, or about 65 mol % of ionizable lipid, or a range between and including any two of the foregoing values. In some embodiments, the lipid nanoparticle compositions described herein comprise about 45 mol % to about 50 mol % of ionizable lipid.

In some embodiments, the ionizable lipid is an ionizable amino lipid. In some embodiments, the ionizable amino lipid may have a positively charged hydrophilic head and a hydrophobic tail that are connected via a linker structure.

In some embodiments, the ionizable lipid is a compound of Formula (IL):

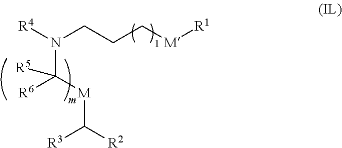

or an N-oxide or a salt thereof, wherein:
R²¹ is

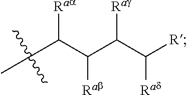

wherein

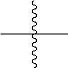

denotes a point of attachment;
R^{aα}, R^{aβ}, R^{aγ}, and R^{aδ} are each independently selected from H, C₂₋₁₂ alkyl, and C₂₋₁₂ alkenyl;
R²² and R²³ are each independently selected from C₁₋₁₄ alkyl and C₂₋₁₄ alkenyl;
R²⁴ is selected from —(CH₂)ₙₙOH and

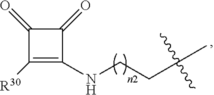

wherein nn is selected from 1, 2, 3, 4, and 5;
wherein

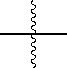

denotes a point of attachment,
wherein R³⁰ is N(R)₂;
wherein each R is independently selected from C₁₋₆ alkyl, C₂₋₃ alkenyl, and H; wherein n2 is selected from 1, 2, 3, 4, 5, 6, 7, 8, 9, and 10;
each R²⁵ is independently selected from C₁₋₃ alkyl, C₂₋₃ alkenyl, and H;
each R²⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
ll is selected from 1, 2, 3, 4, and 5; and
mm is selected from 5, 6, 7, 8, 9, 10, 11, 12, and 13.

In some embodiments, the ionizable lipid is a compound of Formula (IL), or an N-oxide or a salt thereof, wherein R^{aα}, R^{aβ}, R^{aγ}, and R^{aδ} are each H. In some embodiments R^{aα} is H such that R²¹ is

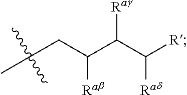

In some embodiments, R^{aα} is C₂₋₁₂ alkyl, and R^{aβ}, R^{aγ}, and R^{aδ} are each H. In some embodiments, R^{aα}, R^{aβ}, and R^{aδ} are each H, and R^{aγ} is C₂₋₁₂ alkyl, e.g., C₂₋₆ alkyl. In some embodiments, R′ is C₁₋₁₂ alkyl, e.g., C₂₋₇ alkyl or C₃₋₈ alkyl.

In some embodiments, the ionizable lipid is a compound of Formula (IL), or an N-oxide or a salt thereof, wherein the combined number of carbons for R²² and R²³ together is at least 6, at least 7, at least 8, at least 9, at least 10, at least 11, at least 12, at least 13, at least 14, at least 15, or at least 16 carbons. In some embodiments, the combined number of carbons for R²² and R²³ together is at least 9 carbons. In some embodiments, R²² and R²³ are independently selected from the group consisting of C₅₋₁₄ alkyl and C₅₋₁₄ alkenyl. In some embodiments, R²² and R²³ are each independently C₅₋₁₄ alkyl. In some embodiments R²² and R²³ are each independently C₆₋₁₀ alkyl. In some embodiments, R²² and R²³ are each C₈ alkyl.

In some embodiments, the ionizable lipid is a compound of Formula (IL), or an N-oxide or a salt thereof, wherein R²⁴ is —(CH₂)ₙₙOH. In some embodiments, nn is 1, 2, 3, or 4. In some embodiment, nn is 2. In some embodiments, R²⁴ is

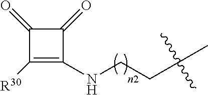

In some embodiments, R³⁰ is —NH(C₁₋₆ alkyl). In some embodiments, R³⁰ is NH(CH₃) In some embodiments, n2 is 1, 2, 3, or 4. In some embodiments, n2 is 2. In some embodiments, R³⁰ is —NH(C₁₋₆ alkyl) (e.g., NH(CH₃)), and n2 is 2.

In some embodiments, the ionizable lipid is a compound of Formula (IL), or an N-oxide or a salt thereof, wherein each R²⁵ is H and/or each R²⁶ is H.

In some embodiments, the ionizable lipid is a compound of Formula (IL), or an N-oxide or a salt thereof, wherein M and M′ are each —C(O)O—.

In some embodiments, the ionizable lipid is a compound of Formula (IL), or an N-oxide or a salt thereof, wherein 1 is 3, 4, or 5. In some embodiments 1 is 5. In some embodiments, m is 5, 6, 7, 8, or 9. In some embodiments, m is 7. In some embodiments, 1 is 3 and m is 7. In certain embodiments, 1 is 5 and m is 7.

In some embodiments, the ionizable lipid is a compound of Formula (IL), or an N-oxide or a salt thereof, wherein:
R¹ is

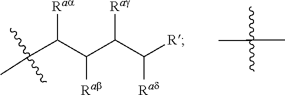

wherein

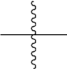

denotes a point of attachment;
R^{aα}, R^{aβ}, R^{aγ}, and R^{aδ} are each H;
R²² and R²³ are each C₁₋₁₄ alkyl;
R²⁴ is —(CH₂)ₙₙOH;
nn is 2;
each R²⁵ is H;
each R²⁶ is H;
M and M′ are each —C(O)O—;
R′ is C₁₋₁₂ alkyl;
ll is 5; and
mm is 7.

In some embodiments, the ionizable lipid is a compound of Formula (IL), or an N-oxide or a salt thereof, wherein:
R²¹ is

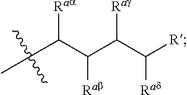

wherein

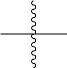

denotes a point of attachment;
R^{aα} is C₂₋₁₂ alkyl;
R^{aβ}, R^{aγ}, and R^{aδ} are each H;
R²² and R²³ are each C₁₋₁₄ alkyl;
R²⁴ is

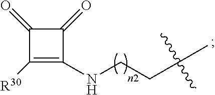

R³⁰ is —NH(C₁₋₆ alkyl);
n2 is 2;
each R²⁵ is H;
each R²⁶ is H;
M and M′ are each —C(O)O—;
R′ is C₁₋₁₂ alkyl;
ll is 5; and
mm is 7.

In some embodiments, the ionizable lipid is a compound of Formula (IL), or an N-oxide or a salt thereof, wherein:
R²¹ is

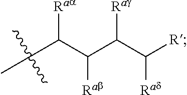

wherein

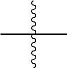

denotes point of attachment;
R^{aα}, R^{aβ}, and R^{aδ} are each H;
R^{aγ} is C₂₋₁₂ alkyl;
R²² and R²³ are each C₁₋₁₄ alkyl;
R²⁴ is —(CH₂)ₙₙOH;
nn is 2;
each R²⁵ is H;
each R²⁶ is H;
M and M′ are each —C(O)O—;
R′ is C₁₋₁₂ alkyl;
ll is 5; and
mm is 7.

In some embodiments, the ionizable lipid is selected from:

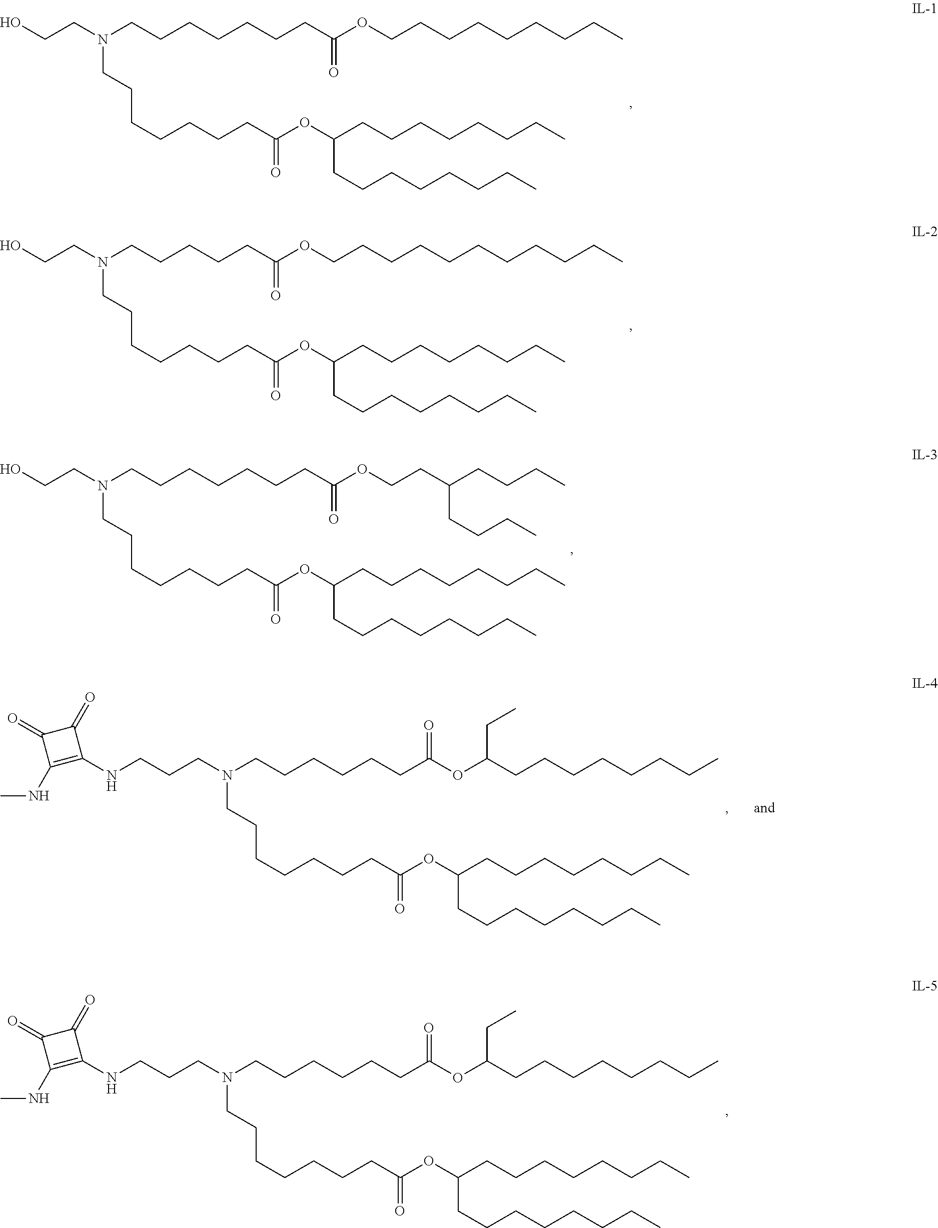

or an N-oxide or a salt thereof.

In some embodiments, the ionizable lipid is a compound of Formula (IL), or an N-oxide or a salt thereof, wherein:
R²¹ is

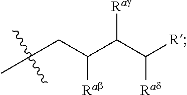

wherein

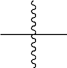

denotes a point of attachment;
R^{aβ}, R^{aγ}, and R^{aδ} are each H;
R²² and R²³ are each C₁₋₁₄ alkyl;
R²⁴ is —(CH₂)ₙₙOH;
nn is 2;
each R²⁵ is H;
each R²⁶ is H;
M and M′ are each —C(O)O—;
R′ is C₁₋₁₂ alkyl;
ll is 3; and
mm is 7.

In some embodiments, the ionizable lipid is a compound of Formula (IL), or an N-oxide or a salt thereof, wherein:
R²¹ is

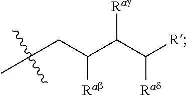

wherein

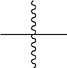

denotes a point of attachment;
R^{aβ} and R^{aδ} are each H;
R^{aγ} is C₂₋₁₂ alkyl;
R²² and R²³ are each C₁₋₁₄ alkyl;
R²⁴ is —(CH₂)ₙₙOH;
nn is 2;
each R²⁵ is H;
each R²⁶ is H;
M and M′ are each —C(O)O—;
R′ is C₁₋₁₂ alkyl;
ll is 5; and
mm is 7.

In some embodiments:
R²¹ is

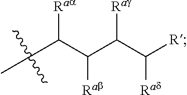

wherein

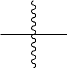

denotes a point of attachment;
R^{aβ}, R^{aγ}, and R^{aδ} are each H;
R^{aα} is C₂₋₁₂ alkyl;
R²² and R²³ are each C₁₋₁₄ alkyl;
R²⁴ is

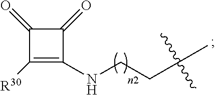

wherein

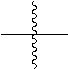

denotes a point of attachment;
wherein R³⁰ is NH(C₁₋₆ alkyl);
wherein n2 is 2;
each R²⁵ is H;
each R²⁶ is H;
M and M′ are each —C(O)O—;
R′ is C₁₋₁₂ alkyl;
ll is 5; and
mm is 7.

In some embodiments:
R²¹ is

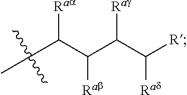

wherein

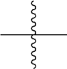

denotes a point of attachment;
R^{aα}, R^{aβ}, and R^{aδ} are each H;
R^{aγ} is C₂₋₁₂ alkyl;
R²² and R²³ are each C₁₋₁₄ alkyl;
R²⁴ is

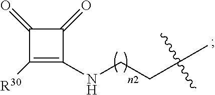

wherein

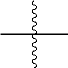

denotes a point of attachment;
wherein R³⁰ is NH(C₁₋₆ alkyl);
wherein n2 is 2;
each R²⁵ is H;
each R²⁶ is H;
M and M′ are each —C(O)O—;
R′ is C₁₋₁₂ alkyl;
ll is 5; and
mm is 7.

The TLA/TLNP may include one or more positively charged ionizable lipids (e.g., lipids that may have a positive or partial positive charge at physiological pH) in addition to an ionizable lipid (e.g., a compound of Formula (IL)) described herein. Cationic and/or ionizable lipids may be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, a cationic lipid may also be a lipid including a cyclic amine group.

Other examples of cationic/ionizable amino lipids can be found in, e.g., U.S. Pat. No. 11,066,355, published Jul. 20, 2021; International PCT Application Publication Nos. WO 2017/049245, published Mar. 23, 2017; WO 2017/112865, published Jun. 29, 2017; WO 2018/170306, published Sep. 20, 2018; WO 2018/232120, published Dec. 20, 2018; WO 2020/061367, published Mar. 26, 2020; WO 2021/055835, published Mar. 25, 2021; WO 2021/055833, published Mar. 25, 2021; WO 2021/055849, published Mar. 25, 2021; and WO 2022/204288, published Sep. 29, 2022, the entire contents of each of which (including any generic or specific structures disclosed therein) is incorporated herein by reference.

In some embodiments, ionizable lipids can be tertiary amine lipids such as described in.

### PEG-Lipids

The TLA and lipid nanoparticle compositions of the present technology optionally include a PEG lipid (e.g., a PEG-lipid described herein). PEG-lipid component of a lipid nanoparticle composition may include one or more molecules comprising polyethylene glycol, such as PEG or PEG-modified lipids. Such species may be alternately referred to as PEGylated lipids. A PEG-lipid is a lipid modified with polyethylene glycol.

In some embodiments, the lipid nanoparticle compositions described herein comprise about 0.15 mol % to about 15 mol % of PEG-lipid, including about 0.15 mol % to about 10 mol %, about 0.15 mol % to about 5 mol %, about 0.15 mol % to about 3 mol %, or about 1 mol % to about 3 mol %. In some embodiments, the nanoparticle described herein comprises about 0.15 mol %, about 0.5 mol %, about 1 mol %, about 1.5 mol %, about 2 mol %, about 2.5 mol %, about 3 mol %, about 3.5 mol %, about 4 mol %, about 4.5 mol %, about 5 mol %, about 10 mol %, or about 15 mol % of PEG-lipid, or a range between and including any two of the foregoing values. In some embodiments, the lipid nanoparticle compositions described herein comprise about 0.15 mol % to about 5 mol % of PEG-lipid. In some embodiments, the lipid nanoparticle compositions described herein comprise about 1 mol % to about 3 mol % of PEG-lipid.

A PEG-lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG-lipid may be PEG-c-DOMG, PEG-DMG (e.g., PEG-DMG 2000 or DMG-PEG 2000), PEG-DLPE, PEG-DMPE, PEG-DPPC, or a PEG-DSPE lipid.

In some embodiments, the PEG-lipid is PEG-DMG (DMG-PEG or 1,2-dimyristoyl-rac-glycero-3-methoxypolyethylene glycol). In some embodiments, the PEG-lipid is PEG-DMG 2000 (or DMG-PEG 2000), where the 2000 represents an average molecular weight. Representative PEG-DMG structures are below.

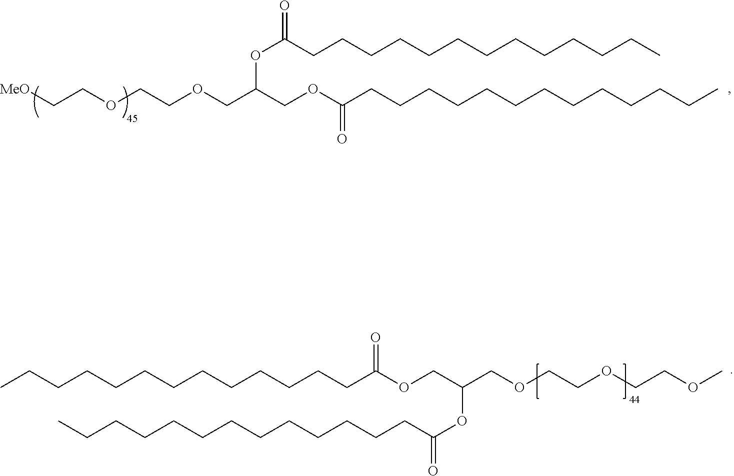

In some embodiments, PEG-lipids can be PEGylated lipids such as described in International Publication Nos. WO 2012/099755 and WO 2017/099823, the contents of each of which is herein incorporated by reference in its entirety. Any of these exemplary PEG-lipids described herein may be modified to comprise a hydroxyl group on the PEG chain. In certain embodiments, the PEG-lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

In certain embodiments, a PEG-lipid is a PEGylated fatty acid. In certain embodiments, a PEG-lipid is a compound of Formula (PGL-I). Provided herein are compounds of Formula (PGL-I):

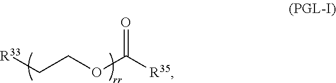

or a salts thereof, wherein:
R³³ is —OR^{O};
R^{O} is hydrogen, optionally substituted alkyl or an oxygen protecting group;
rr is an integer between 1 and 100, inclusive;
R³⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R³⁵ are replaced with optionally substituted carbocyclylene, optionally substituted heterocyclylene, optionally substituted arylene, optionally substituted heteroarylene, —N(R^{N})—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—,
   —S(O)N(R^{N})—, —N(R^{N})S(O)N(R^{N})—, —OS(O)N(R^{N})—, —N(R^{N})S(O)O—, —S(O)₂—, —N(R^{N})S(O)₂—,
   —S(O)₂N(R^{N})—, —N(R^{N})S(O)₂N(R^{N})—, —OS(O)₂N(R^{N})—, or —N(R^{N})S(O)₂O—; and
each instance of R^{N} is independently hydrogen, optionally substituted alkyl, or a nitrogen protecting group.

In certain embodiments, the compound of Formula (PGL-I) is of Formula (PGL-I-OH):

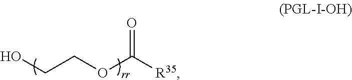

or a salt thereof.

In certain embodiments, a compound of Formula (PGL-I) is of one of the following formulae:

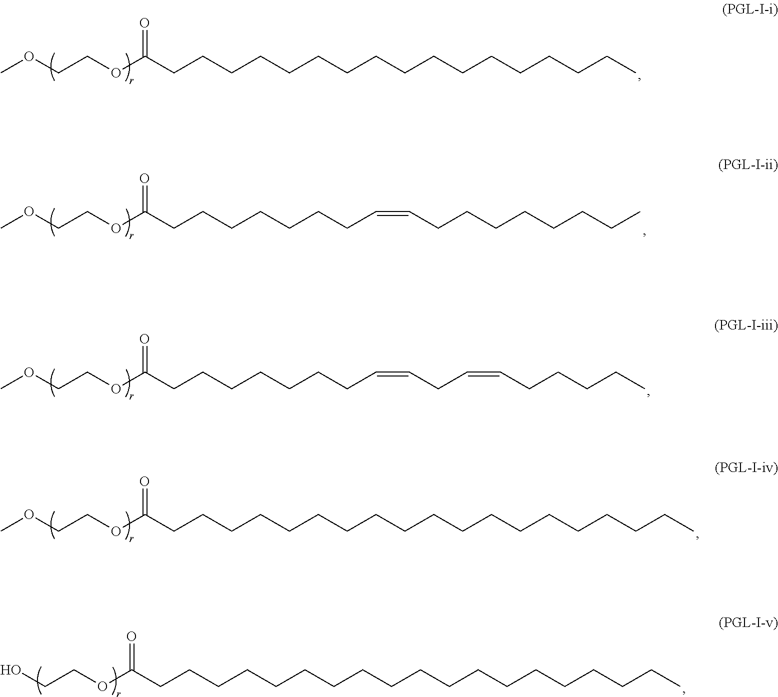

or a salt thereof. In some embodiments, rr is 43, 44, 45, or 46. In some embodiments, rr is 45.

In yet other embodiments the compound of Formula (PGL-I) has the formula:

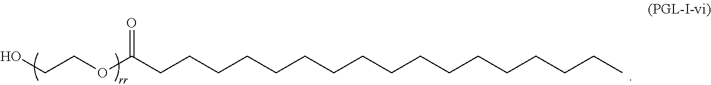

In some embodiments, the compound of Formula (PGL-I) is

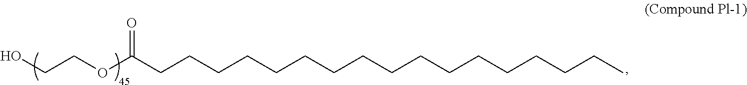

or In certain embodiments, certain embodiments, the PEG-lipid is one of the following formula:

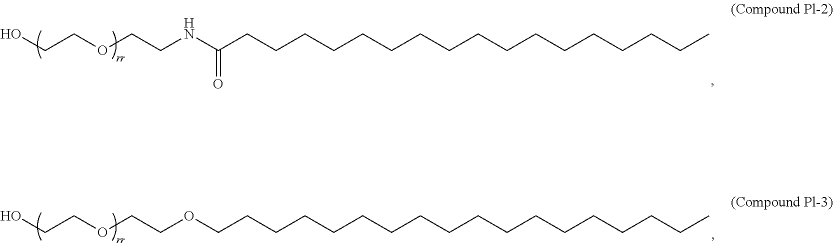

or a salt thereof. In some embodiments, rr is 45.

Suitable additional PEG-lipids are described in WO 2017/099823 which is herein incorporated by reference in its entirety.

### Phospholipids

The TLA and lipid nanoparticle compositions of the present technology optionally include a phospholipid. Phospholipids, as defined herein, are lipids that comprise a phosphate group. The lipid component of a lipid nanoparticle composition may include one or more phospholipids, such as one or more (poly)unsaturated lipids. Phospholipids may assemble into one or more lipid bilayers. In general, phospholipids may include a phospholipid moiety and one or more fatty acid moieties. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

In some embodiments, the lipid nanoparticle compositions described herein can comprise about 5 mol % to about 25 mol % of phospholipid, including about 5 mol % to about 15 mol %, or about 8 mol % to about 13 mol %. In some embodiments, the nanoparticle described herein comprises about 5 mol %, about 7.5 mol %, about 10 mol %, about 12.5 mol %, about 15 mol %, about 20 mol %, or about 25 mol %, of phospholipid, or a range between and including any two of the foregoing values. In some embodiments, the lipid nanoparticle composition comprises about 8 mol % to about 13 mol % of phospholipid. In some embodiments, the lipid nanoparticle composition comprises about 10 mol % to about 12 mol % of phospholipid.

Suitable phospholipids include:
1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-diphytanoyl-sn-glycero-3-phosphoethanolamine (ME 16.0 PE),
1,2-diphytanoyl-sn-glycero-3-phosphocholine (4ME 16:0 PC),
1,2-diphytanoyl-sn-glycero-3-phospho-(1′-rac-glycerol) (sodium salt) (4ME 16:0 PG),
1,2-diphytanoyl-sn-glycero-3-phospho-L-serine (sodium salt) (4ME 16:0 PS),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), and sphingomyelin.

Each possibility represents a separate embodiment.

In some embodiments, the phospholipid is selected from the group consisting of DOMG, DMG, DLPE, DMPE, DPPC, DSPE and a combination of any two or more thereof. In some embodiments, the phospholipid is DSPC. In certain embodiments, the phospholipid is DOPE. In some embodiments, the phospholipid includes both DSPC and DOPE.

Examples of suitable phospholipids include, but are not limited to, the following:

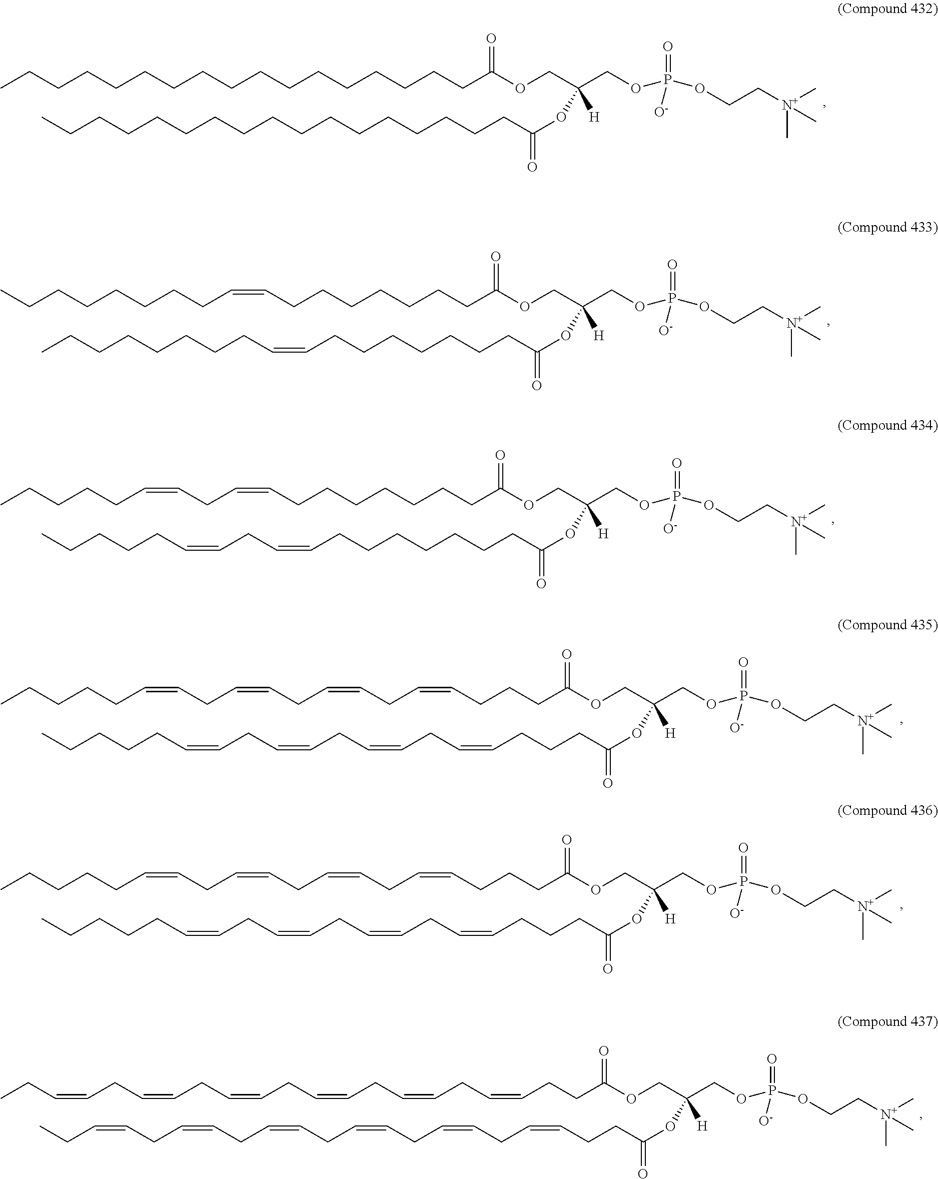

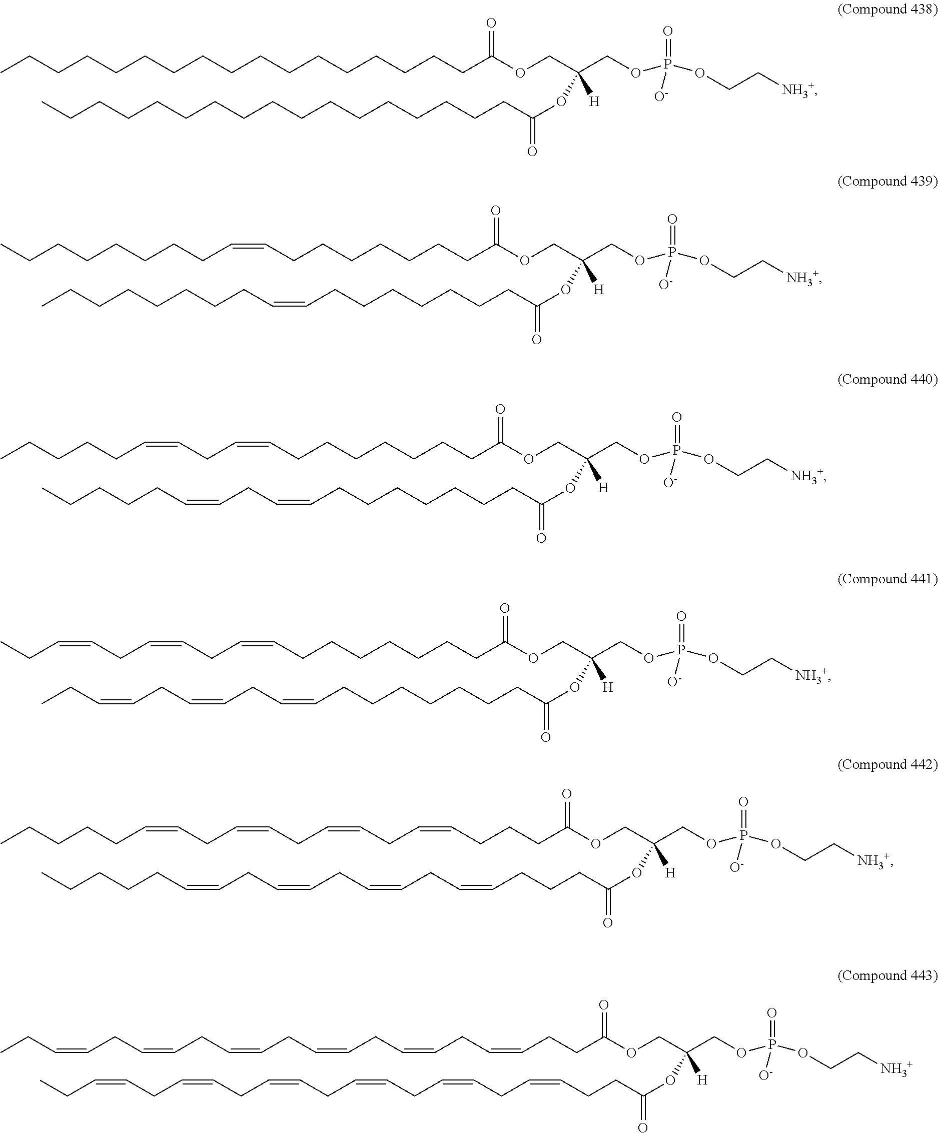

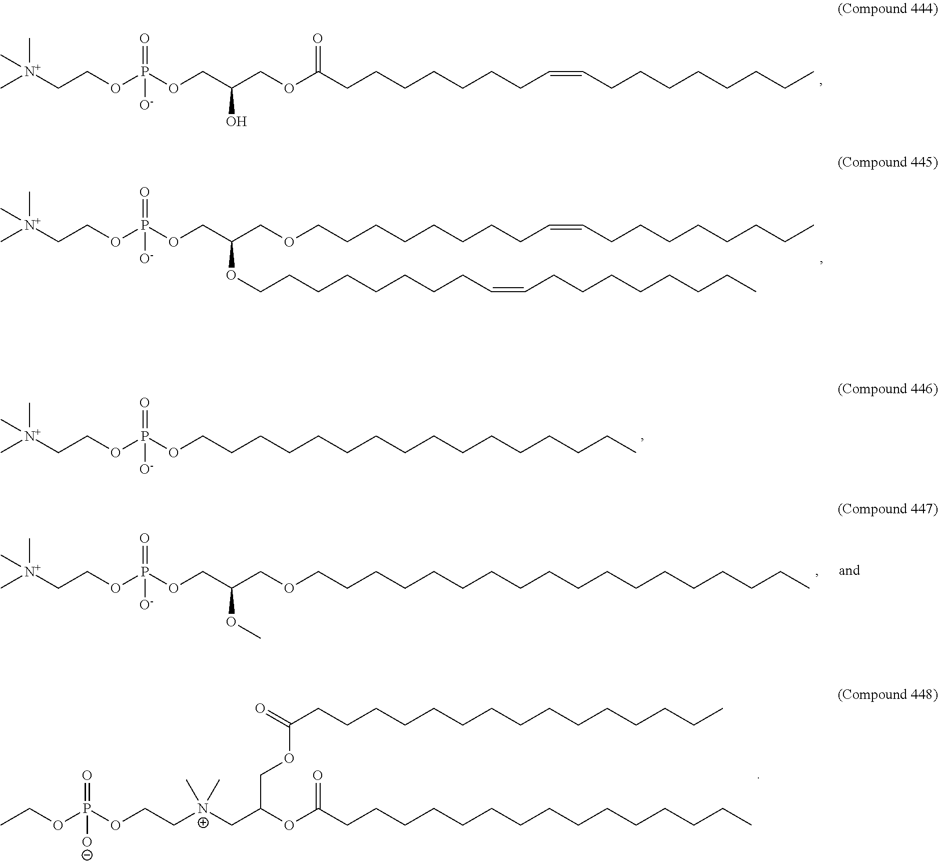

In certain embodiments, a phospholipid is a compound of Formula (PHL-I):

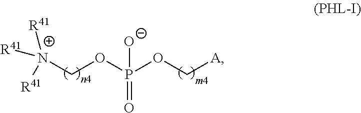

or a salt thereof, wherein:
each R⁴¹ is independently H or optionally substituted alkyl; or optionally two R⁴¹ are joined together with the intervening atoms to form optionally substituted monocyclic carbocyclyl or optionally substituted monocyclic heterocyclyl; or optionally three R⁴¹ are joined together with the intervening atoms to form optionally substituted bicyclic carbocyclyl or optionally substitute bicyclic heterocyclyl;
n4 is 1, 2, 3, 4, 5, 6, 7, 8, 9, or 10;
m4 is 0, 1, 2, 3, 4, 5, 6, 7, 8, 9, or 10;
A is of the formula:

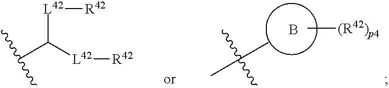

each instance of L⁴² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R⁴² is independently optionally substituted C₁₋₃₀ alkyl, optionally substituted C₁₋₃₀ alkenyl, or optionally substituted C₁₋₃₀ alkynyl; optionally wherein one or more methylene units of R⁴² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)₂N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p4 is 1 or 2;
provided that the compound is not of the formula:

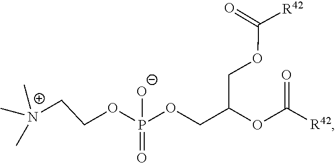

wherein each instance of R⁴² is independently unsubstituted alkyl, unsubstituted alkenyl, or unsubstituted alkynyl.

In certain embodiments, a suitable phospholipid is an analog or variant of DSPC such as a compound of Formula (PHL-I):

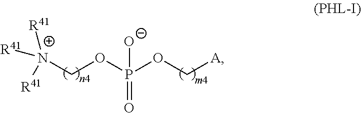

or a salt thereof, wherein:
each R⁴¹ is independently optionally substituted alkyl; or optionally two R⁴¹ are joined together with the intervening atoms to form optionally substituted monocyclic carbocyclyl or optionally substituted monocyclic heterocyclyl; or optionally three R⁴¹ are joined together with the intervening atoms to form optionally substituted bicyclic carbocyclyl or optionally substitute bicyclic heterocyclyl;
n4 is 1, 2, 3, 4, 5, 6, 7, 8, 9, or 10;
m4 is 0, 1, 2, 3, 4, 5, 6, 7, 8, 9, or 10;
A is of the formula:

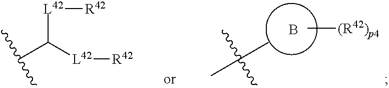

each instance of L⁴² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R⁴² is independently optionally substituted C₁₋₃₀ alkyl, optionally substituted C₁₋₃₀ alkenyl, or optionally substituted C₁₋₃₀ alkynyl; optionally wherein one or more methylene units of R⁴² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p4 is 1 or 2.
provided that the compound is not of the formula:

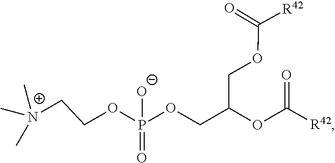

wherein each instance of R⁴² is independently unsubstituted alkyl, unsubstituted alkenyl, or unsubstituted alkynyl.

In some embodiments, the compound is not of the formula:

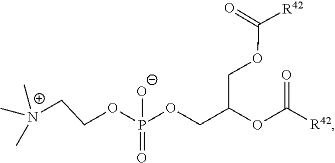

wherein each instance of R⁴² is independently unsubstituted alkyl, unsubstituted alkenyl, or unsubstituted alkynyl.

In certain embodiments, a suitable phospholipid comprises a modified phospholipid head (e.g., a modified choline group). In certain embodiments, a phospholipid with a modified head is DSPC, or analog thereof, with a modified quaternary amine. For example, in embodiments of Formula (PHL-I), at least one of R⁴¹ is not methyl. In certain embodiments, at least one of R⁴¹ is not hydrogen or methyl. In certain embodiments, the compound of Formula (PHL-I) is of one of the following formulae:

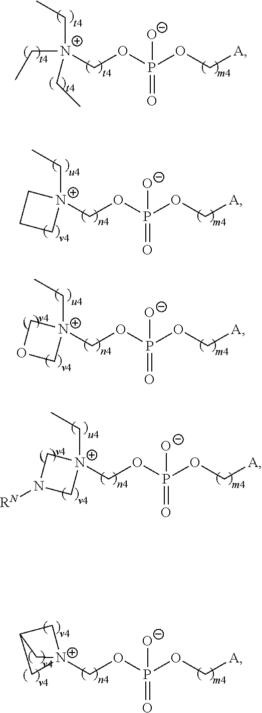

or a salt thereof, wherein:
each t4 is independently 1, 2, 3, 4, 5, 6, 7, 8, 9, or 10;
each u4 is independently 0, 1, 2, 3, 4, 5, 6, 7, 8, 9, or 10; and
each v4 is independently 1, 2, or 3.

In certain embodiments, the compound of Formula (PHL-I) is of one of the following formulae:

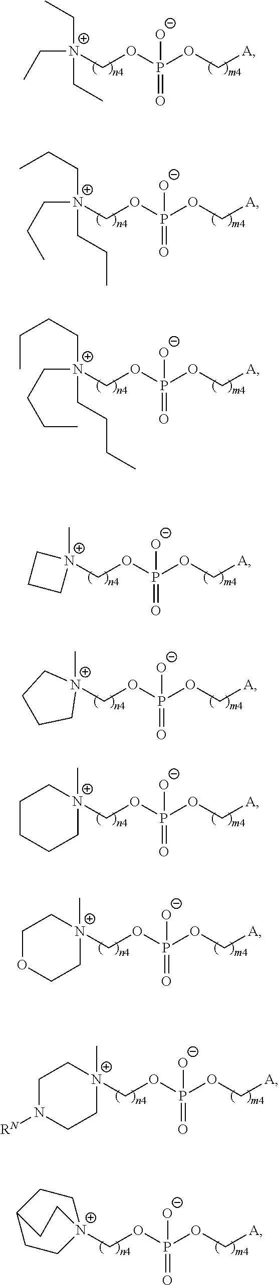

or a salt thereof.

In certain embodiments, a compound of Formula (PHL-I) is one of the following:

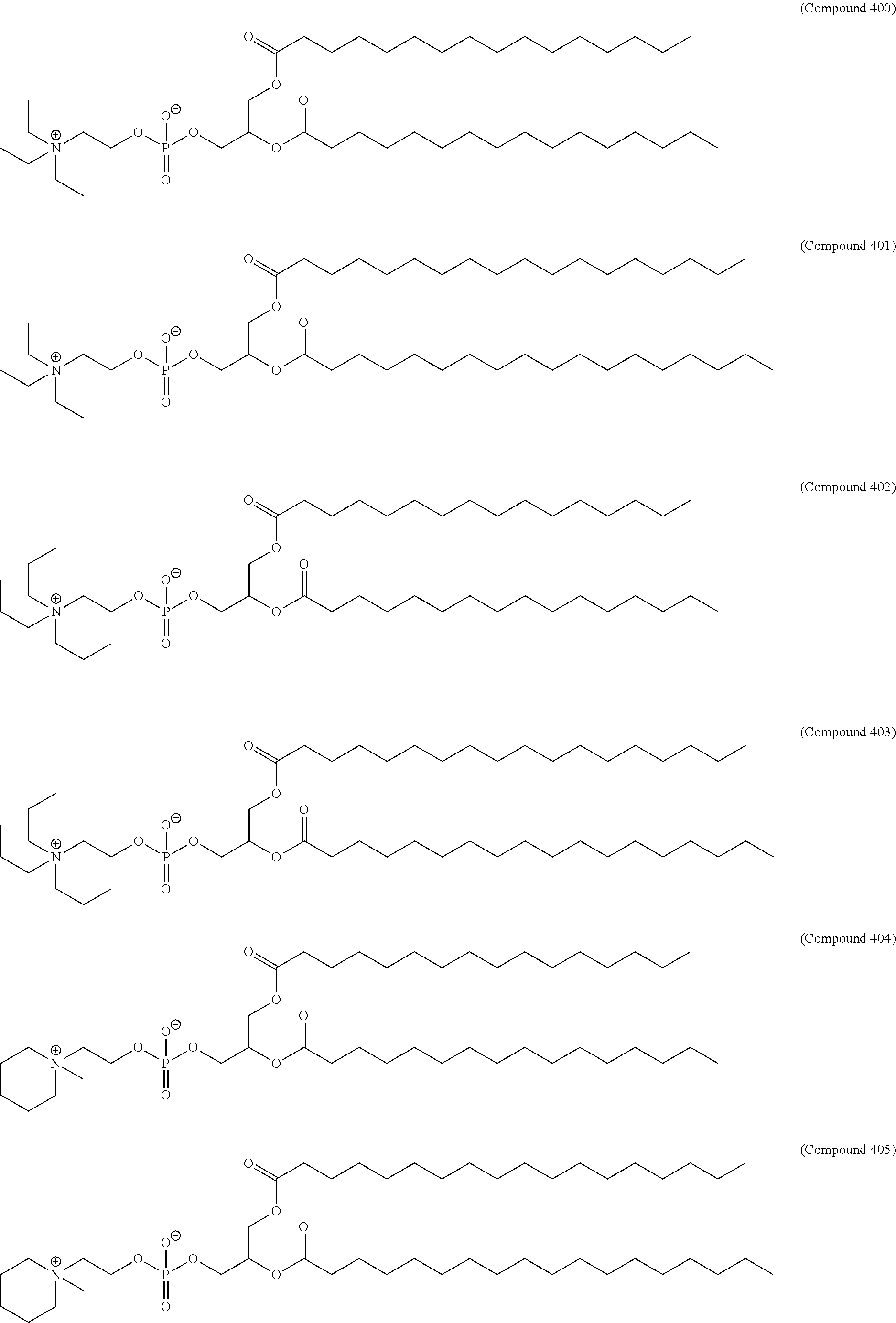

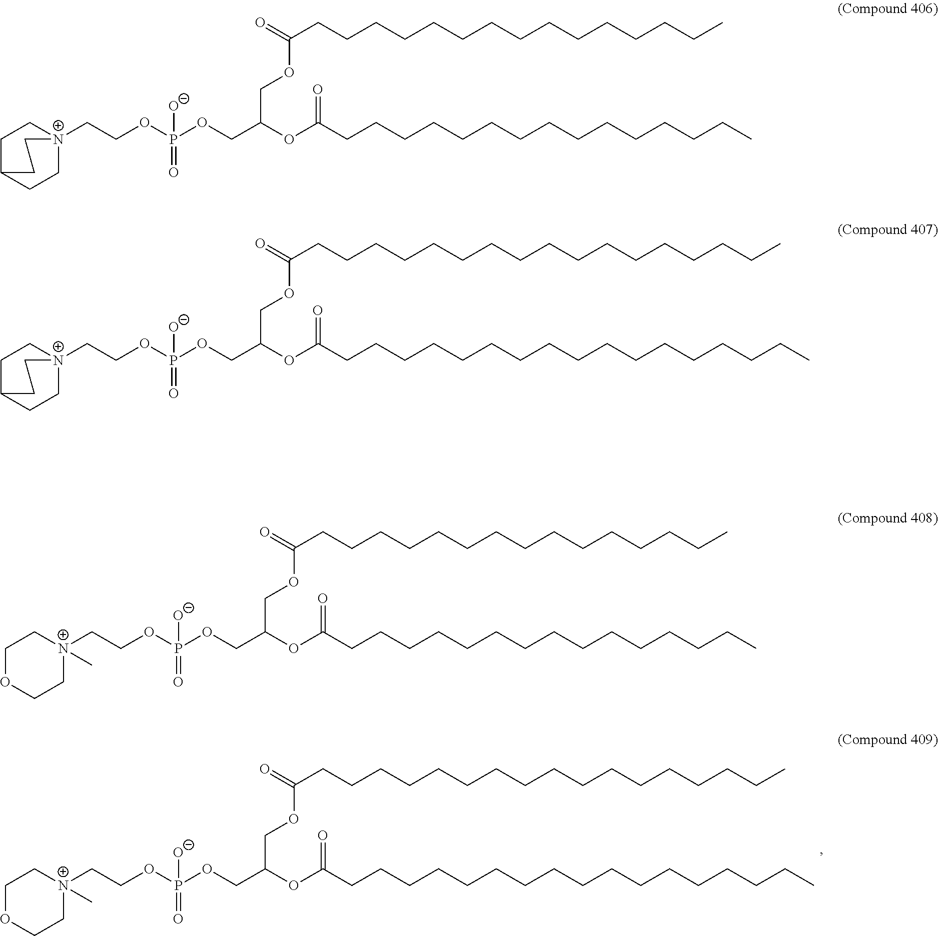 [SMILES: CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In certain embodiments, a compound of Formula (PHL-I) is of Formula (PHL-I-a):

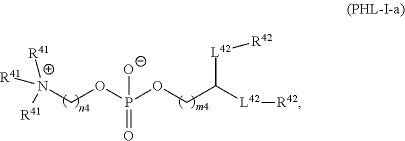

or a salt thereof.

In certain embodiments, suitable phospholipids comprise a modified core. In certain embodiments, a phospholipid with a modified core described herein is DSPC, or analog thereof, with a modified core structure. For example, in certain embodiments of Formula (PHL-I-a), group A is not of the following formula:

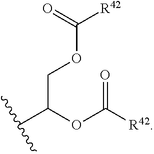

In certain embodiments, the compound of Formula (PHL-I-b-4) is one of the following formulae:

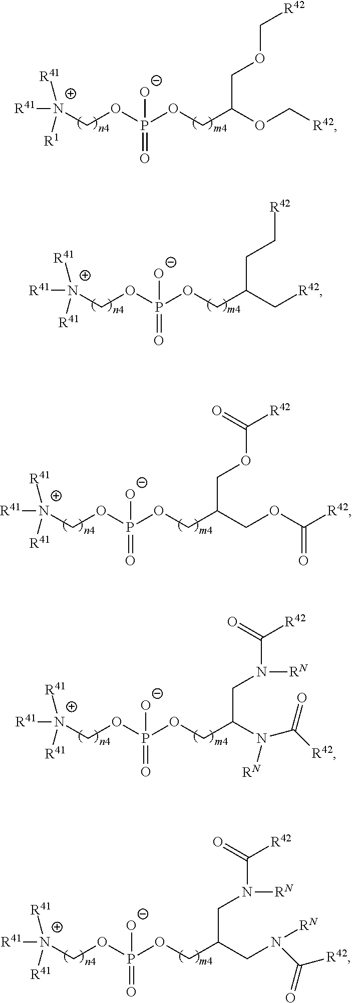

or a salt thereof.

In certain embodiments, a suitable phospholipid comprises a modified phosphocholine moiety, wherein the alkyl chain linking the quaternary amine to the phosphoryl group is not ethylene (e.g., n4 is not 2). Therefore, in certain embodiments, a phospholipid is a compound of Formula (PHL-I), wherein n is 1, 3, 4, 5, 6, 7, 8, 9, or 10. For example, in certain embodiments, a compound of Formula (PHL-I) is of one of the following formulae:

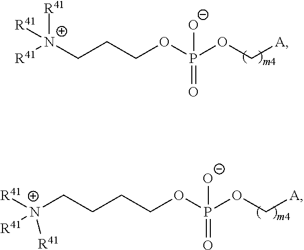

or a salt thereof.

In certain embodiments, a compound of Formula (PHL-I) is one of the following:

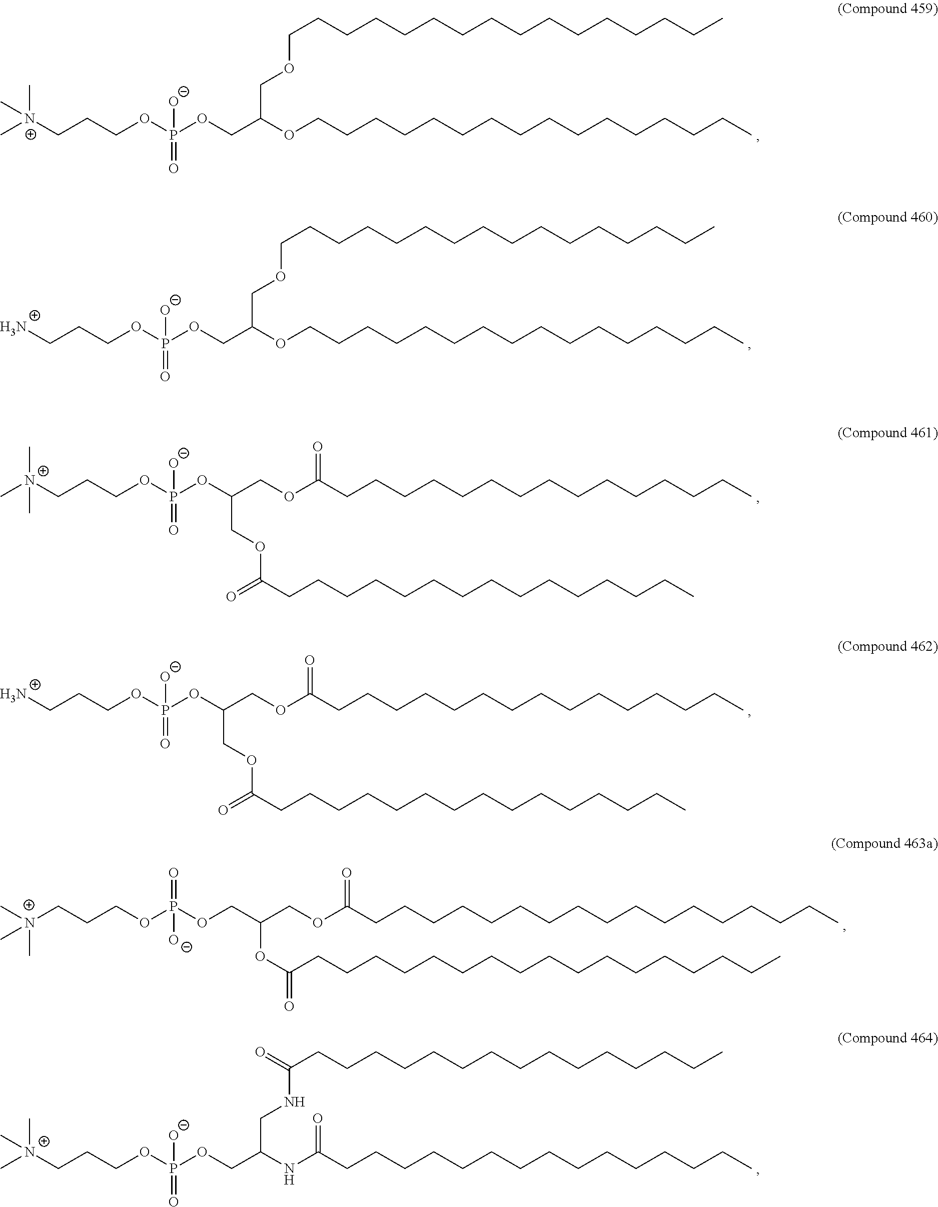

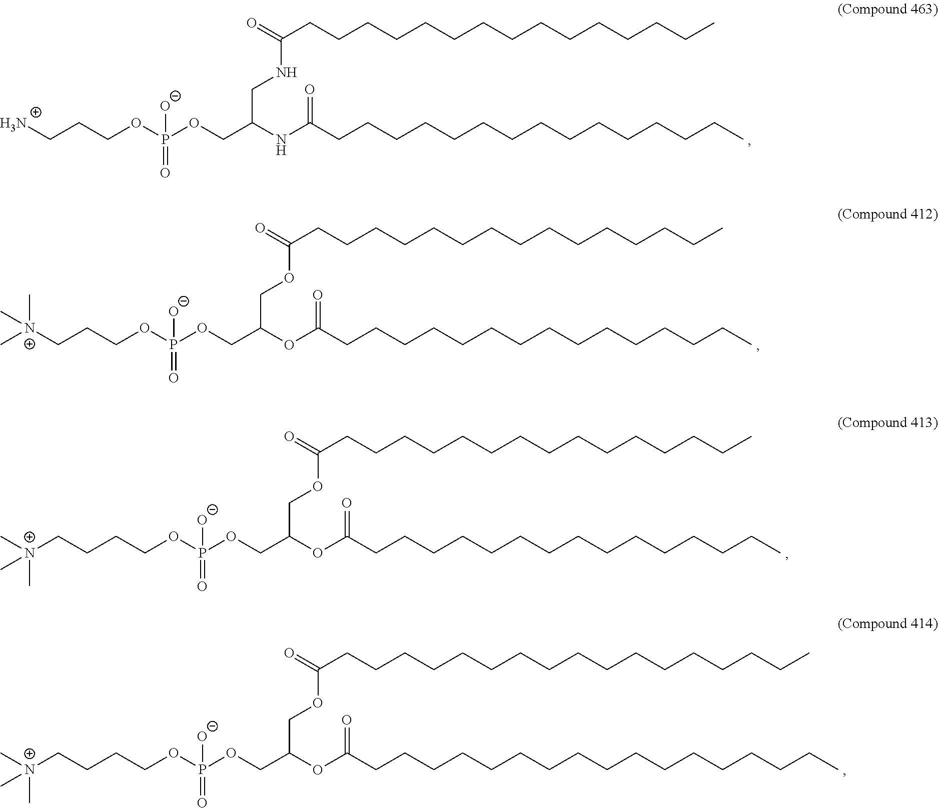

or a salt thereof.

### Structural Lipids

The TLA and lipid nanoparticle compositions of the present technology may include one or more structural lipids. As used herein, the term “structural lipid” refers to sterols and also to lipids containing sterol moieties. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

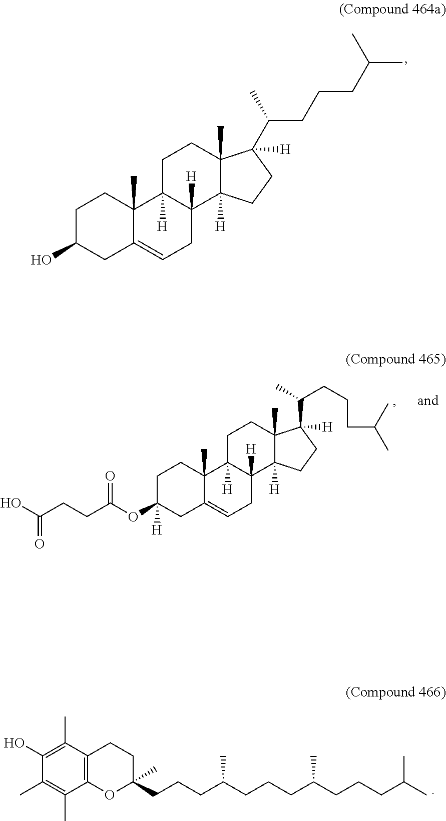

In some embodiments, the structural lipid is selected from the group consisting of a sterol, a tocopherol, and mixtures of two or more thereof. In some embodiments, the structural lipid is selected from the group consisting of cholesterol and alpha-tocopherol.

In some embodiments, the lipid nanoparticle compositions described herein can comprise about 20 mol % to about 60 mol % structural lipid, including about 30 mol % to about 50 mol %. In some embodiments, the lipid nanoparticle compositions comprise about 15 mol % to about 45 mol % of structural lipid. In some embodiments, the lipid nanoparticle compositions comprise about 35 mol % to about 45 mol % of structural lipid. In some embodiments, the lipid nanoparticle compositions comprise about 37 mol % to about 42 mol % of structural lipid. In some embodiments, the lipid nanoparticle compositions comprise about 20 mol %, 25 mol %, 30 mol %, 35 mol %, 40 mol %, 45 mol %, 50 mol %, 55 mol %, or 60 mol %, of structural lipid, or a range between and including any two of the foregoing values. In some embodiments, the lipid nanoparticle compositions comprise about 35, about 36, about 37, about 38, about 39, or about 40 mol % of structural lipid. In some embodiments, the nanoparticle comprises about 39 to about 40 mol % structural lipid. In some embodiments, the structural lipid is cholesterol or a compound having the following structure:

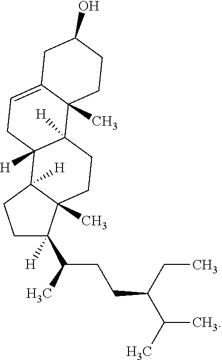

### Therapeutic and Prophylactic Agents as Payloads

The TLA and lipid nanoparticle compositions of the disclosure can be used to deliver a wide variety of different payloads to a subject, including therapeutic agents to a patient. It is to be understood that reference to therapeutic agents is a reference to “therapeutic and/or prophylactic agents” unless otherwise indicated. Typically, the payload delivered by the composition is a nucleic acid, although non-nucleic acid agents, such as small molecules, chemotherapy drugs, peptides, polypeptides, and other biological molecules are also payloads encompassed by the disclosure. Nucleic acids that can be delivered include DNA-based molecules (i.e., comprising deoxyribonucleotides) and RNA-based molecules (i.e., comprising a ribonucleotides). Furthermore, the nucleic acid can be a naturally occurring form of the molecule or a chemically-modified form of the molecule (e.g., comprising one or more modified nucleotides).

In some embodiments, the therapeutic agent is an agent that enhances (i.e., increases, stimulates, upregulates) protein expression. Non-limiting examples of types of therapeutic agents that can be used for enhancing protein expression include RNAs, mRNAs, dsRNAs, CRISPR/Cas9 technology, ssDNAs and DNAs (e.g., expression vectors).

In some embodiments, the therapeutic agent is a DNA therapeutic agent. The DNA molecule can be a double-stranded DNA, a single-stranded DNA (ssDNA), or a molecule that is a partially double-stranded DNA, i.e., has a portion that is double-stranded and a portion that is single-stranded. In some cases the DNA molecule is triple stranded or is partially triple-stranded, i.e., has a portion that is triple stranded and a portion that is double stranded. The DNA molecule can be a circular DNA molecule or a linear DNA molecule.

A DNA therapeutic agent can be a DNA molecule that is capable of transferring a gene into a cell, e.g., that encodes and can express a transcript. In some embodiments, the DNA molecule can be naturally-derived, e.g., isolated from a natural source. In other embodiments, the DNA molecule is a synthetic molecule, e.g., a synthetic DNA molecule produced in vitro. In some embodiments, the DNA molecule is a recombinant molecule. Non-limiting exemplary DNA therapeutic agents include plasmid expression vectors and viral expression vectors.

The DNA therapeutic agents described herein, e.g., DNA vectors, can include a variety of different features. The DNA therapeutic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

In some embodiments, the therapeutic agent is an RNA therapeutic agent. The RNA molecule can be a single-stranded RNA, a double-stranded RNA (dsRNA) or a molecule that is a partially double-stranded RNA, i.e., has a portion that is double-stranded and a portion that is single-stranded. The RNA molecule can be a circular RNA molecule or a linear RNA molecule.

An RNA therapeutic agent can be an RNA therapeutic agent that is capable of transferring a gene into a cell, e.g., encodes a protein of interest, to thereby increase expression of the protein of interest in a mammalian target cell. In some embodiments, the RNA molecule can be naturally-derived, e.g., isolated from a natural source. In other embodiments, the RNA molecule is a synthetic molecule, e.g., a synthetic RNA molecule produced in vitro.

Non-limiting examples of RNA therapeutic agents include messenger RNAs (mRNAs) (e.g., encoding a protein of interest), modified mRNAs (mmRNAs), mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA), locked nucleic acids (LNAs) and CRISPR/Cas9 technology, each of which is described further below.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a nonnaturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyluridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine(mnm5se2U), 5-carbamoylmethyl-uridine (ncm5U), 5-carboxymethylaminomethyluridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(rm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thiopseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thiopseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-Omethyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-Omethyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-Epropenylamino)]uridine.

In some embodiments, the modified nucleobase is a modified cytosine. Exemplary nucleobases and nucleosides having a modified cytosine include 5-azacytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methylpseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thiozebularine, 2-methoxy-cytidine, 2-methoxy-5-methyl-cytidine, 4-methoxypseudoisocytidine, 4-methoxy-1-methyl-pseudoisocytidine, lysidine (k2C), α-thiocytidine, 2′-O-methyl-cytidine (Cm), 5,2′-O-dimethyl-cytidine (m5Cm), N4-acetyl-2′-Omethyl-cytidine (ac4Cm), N4,2′-O-dimethyl-cytidine (m4Cm), 5-formyl-2′-O-methylcytidine (f5Cm), N4,N4,2′-O-trimethyl-cytidine (m42Cm), 1-thio-cytidine, 2′-F-aracytidine, 2′-F-cytidine, and 2′-OH-ara-cytidine.

In some embodiments, the modified nucleobase is a modified adenine. Exemplary nucleobases and nucleosides having a modified adenine include a-thioadenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azidoadenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyladenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-Omethyl-adenosine (Am), N6,2′-O-dimethyl-adenosine (m6Am), N6,N6,2′-O-trimethyladenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-aminopentaoxanonadecyl)-adenosine.

In some embodiments, the modified nucleobase is a modified guanine. Exemplary nucleobases and nucleosides having a modified guanine include a-thioguanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methylguanosine (m2,7Gm), 2′-O-methyl-inosine (Im), 1,2′-O-dimethyl-inosine (m1Im), 2′-Oribosylguanosine (phosphate) (Gr(p)), 1-thio-guanosine, 06-methyl-guanosine, 2′-F-araguanosine, and 2′-F-guanosine.

In some embodiments, an mRNA of the disclosure includes a combination of one or more of the aforementioned modified nucleobases (e.g., a combination of 2, 3, or 4 of the aforementioned modified nucleobases).

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thiopseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3, or 4 of the aforementioned modified nucleobases). In some embodiments, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine. Exemplary nucleobases and nucleosides having a modified cytosine include N4-acetylcytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3, or 4 of the aforementioned modified nucleobases).

In some embodiments, the modified nucleobase is a modified adenine. Exemplary nucleobases and nucleosides having a modified adenine include 7-deazaadenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3, or 4 of the aforementioned modified nucleobases).

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methylguanosine (m7G), 1-methyl-guanosine (m1G), 8-oxo-guanosine, 7-methyl-8-oxoguanosine. In some embodiments, an mRNA of the disclosure includes a combination of one or more of the aforementioned modified nucleobases (e.g., a combination of 2, 3, or 4 of the aforementioned modified nucleobases).

In some embodiments, the modified nucleobase is 1-methyl-pseudouridine (m1ψ), 5-methoxy-uridine (mo5U), 5-methyl-cytidine (m5C), pseudouridine (ψ), α-thioguanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methylcytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mRNAs of the present disclosure include, but are not limited to, those described in PCT Patent Application Publications: WO2012045075, WO2014081507, WO2014093924, WO2014164253, and WO2014159813.

The mRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In some embodiments, the mRNA sequence is optimized using optimization algorithms, e.g., to optimize expression in mammalian cells or enhance mRNA stability.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In some embodiments, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments, the payload therapeutic agent is a therapeutic agent that reduces (i.e., decreases, inhibits, downregulates) protein expression. Non-limiting examples of types of therapeutic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

In some embodiments, the therapeutic agent is a peptide therapeutic agent. In some embodiments the therapeutic agent is a polypeptide therapeutic agent.

In some embodiments, the peptide or polypeptide is naturally-derived, e.g., isolated from a natural source. In other embodiments, the peptide or polypeptide is a synthetic molecule, e.g., a synthetic peptide or polypeptide produced in vitro. In some embodiments, the peptide or polypeptide is a recombinant molecule. In some embodiments, the peptide or polypeptide is a chimeric molecule. In some embodiments, the peptide or polypeptide is a fusion molecule. In some embodiments, the peptide or polypeptide therapeutic agent of the composition is a naturally occurring peptide or polypeptide. In some embodiments, the peptide or polypeptide therapeutic agent of the composition is a modified version of a naturally occurring peptide or polypeptide (e.g., contains less than 3, less than 5, less than 10, less than 15, less than 20, or less than 25 amino substitutions, deletions, or additions compared to its wild-type, naturally occurring peptide or polypeptide counterpart).

### Genome Editing Techniques

In some embodiments, the nucleic acid is suitable for a genome editing technique. In some embodiments, the genome editing technique is clustered regularly interspaced short palindromic repeats (CRISPR) or transcription activator-like effector nuclease (TALEN). In some embodiments, the nucleic acid is at least one nucleic acid suitable for a genome editing technique selected from the group consisting of a CRISPR RNA (crRNA), a trans-activating crRNA (tracrRNA), a single guide RNA (sgRNA), and a DNA repair template. In some embodiments, the TLA and lipid nanoparticle compositions of the disclosure may further include one or more of the CRISPR related proteins (e.g., Cas9, dCas9, or any other CRISPR related protein described herein or known in the art) or a polynucleotide (e.g., an mRNA) encoding the same. The CRISPR related protein can be from any number of species including but not limited to Streptococcus pyogenes, Listeria innocua, and Streptococcus thermophilus. The polynucleotide can encode the wild-type sequence of the CRISPR related protein or a variant CRISPR related protein. CRISPR-associated proteins for use in conjunction with the compositions and methods of the disclosure also include catalytically inactive Cas proteins, such as dCas9. Accordingly, the term “CRISPR related protein” refers to Cas proteins, such as (without limitation) Cas9, CSY4, dCas9, and dCas9-effector domain (activator and/or inhibitor domain) fusion proteins.

### Other Components

A TLA may include one or more components in addition to those described in the preceding sections. In some embodiments, a TLA may include one or more small hydrophobic molecules such as a vitamin (e.g., vitamin A or vitamin E) or a sterol.

Lipid assemblies may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a TLA. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. In some embodiments, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 04, dornase alfa, neltenexine, and erdosteine), and DNases (e.g., rhDNase). A surface altering agent may be disposed within a nanoparticle and/or on the surface of a TLA (e.g., by coating, adsorption, covalent linkage, or other process).

A TLA may also comprise one or more functionalized lipids. In some embodiments, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a TLA may also be conjugated with one or more useful antibodies. Functional groups and conjugates useful in targeted cell delivery, imaging, and membrane permeation are well known in the art.

In addition to these components, lipid assemblies may include any substance useful in pharmaceutical compositions. In some embodiments, the TLA may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{St} Edition, A. R. Gennaro; Lippincott, Williams & Wilkins, Baltimore, MD, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Methods of Preparing the Targeted Lipid Assemblies

In some aspects, the present disclosure provides a method of preparing the population of lipid assemblies described herein.

In some embodiments, the method comprises: i) mixing an ionizable lipid, a structural lipid, a PEG targeting compound and a phospholipid, with a first buffer, thereby forming a population of intermediate empty lipid assemblies.

In some embodiments, the method comprises: i) mixing an ionizable lipid, a structural lipid, a phospholipid, a PEG targeting compound and a PEG lipid, with a first buffer, thereby forming a population of intermediate empty lipid assemblies.

In some embodiments, the method further comprises: ii) adding a second buffer to the intermediate empty lipid assemblies, thereby forming a population of empty lipid assemblies.

In some embodiments, the method further comprises: iii) mixing a therapeutic agent (e.g., a nucleic acid) with the empty-lipid assemblies, thereby forming a population of filled lipid assemblies.

In some embodiments, the method further comprises processing the empty lipid assemblies or the filled lipid assemblies.

In some embodiments, the step of processing comprises:
a) adding a cryoprotectant to the empty lipid assemblies or the filled lipid assemblies;
b) lyophilizing the empty lipid assemblies or the filled lipid assemblies;
c) storing the lyophilized empty lipid assemblies or the lyophilized filled lipid assemblies; and/or
d) adding a buffering solution to the lyophilized empty lipid assemblies or the lyophilized filled lipid assemblies.

Suitable methods for preparing the population of lipid assemblies described herein are also described in PCT Application Publication No. WO/2020/160397, WO/2021/155274, and WO/2022/032087, each of which is incorporated herein by reference.

### Pharmaceutical Compositions

The present disclosure provides pharmaceutical compositions that comprise any of the TLA or lipid nanoparticle compositions described herein together with one or more pharmaceutically acceptable excipients.

Pharmaceutical compositions can optionally comprise one or more additional active substances, e.g., therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21^{st} ed., Lippincott Williams & Wilkins, 2005 (incorporated herein by reference in its entirety). In some embodiments, compositions are administered to humans, human patients or subjects. For the purposes of the present disclosure, the phrase “active ingredient” generally refers to the nanoparticle comprising the polynucleotides or polypeptide payload to be delivered as described herein.

Pharmaceutical compositions described herein can be prepared by any method known or hereafter developed in the art of pharmacology. In general, such preparatory methods include the step of associating the nanoparticle with an excipient and/or one or more other accessory ingredients, and then, if necessary and/or desirable, dividing, shaping and/or packaging the product into a desired single- or multi-dose unit.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject to which the compositions are administered and further depending upon the route by which the composition is to be administered.

Although the descriptions of pharmaceutical compositions provided herein are principally directed to pharmaceutical compositions that are suitable for administration to humans, it will be understood by the skilled artisan that such compositions are generally suitable for administration to any other animal, e.g., to non-human animals, e.g. non-human mammals.

A pharmaceutically acceptable excipient, as used herein, includes, but is not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006; incorporated herein by reference in its entirety).

Oxidation is a potential degradation pathway for mRNA, especially for liquid mRNA formulations. In order to prevent oxidation, antioxidants can be added to the formulations. Exemplary antioxidants include, but are not limited to, alpha tocopherol, ascorbic acid, acorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

The pharmaceutical compositions can be administered in an effective amount to cause a desired biological effect, e.g., a therapeutic or prophylactic effect, e.g., owing to expression of a normal gene product to supplement or replace a defective protein or to reduce expression of an undesired protein. The formulations may be administered in an effective amount to deliver TLNP.

Pharmaceutical compositions may be prepared in a variety of forms suitable for a variety of routes and methods of administration. In some embodiments, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed includingsynthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

The pharmaceutical composition may be prepared, packaged, and/or sold in a formulation suitable for pulmonary administration. Such a formulation may comprise dry particles which comprise the active ingredient. Such compositions can be in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and can be provided in a unit dose form.

Low boiling propellants generally include liquid propellants having a boiling point of below about 65° F. at atmospheric pressure. The propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 □m to 500 □m. Such a formulation is administered in the manner by rapid inhalation through the nasal passage from a container of the powder held close to the nose. Formulations suitable for nasal administration may, for example, comprise from about as little as 0.1% (wt/wt) and as much as 100% (wt/wt) of active ingredient, and may comprise one or more of the additional ingredients described herein.

### Methods of Delivering Therapeutic Agents to Cells and Organs

In another aspect, there are provided methods of specifically delivering a payload such as a therapeutic and/or prophylactic agent to a target cell. The methods may include contacting the target cell with a filled TLA composition (i.e., one including a therapeutic and/or prophylactic agent) as described herein, e.g., a filled TLNP composition. The target cell can be part of an in vitro or ex vivo sample. The target cell can also present within a subject, i.e., within a patient. The target cell may be part of a tissue or organ. In some embodiments, the target cell is a mammalian cell, e.g., a hepatocyte. In some embodiments, the tissue comprises hepatocytes. The target cell may be part of the liver. Thus, the methods may also include administering to a subject any of the TLAs disclosed herein which include the therapeutic and/or prophylactic agent, e.g., those TLAs comprising a compound of any one of Formulas I, IA, IB, IC, ID, and IE, or a pharmaceutical composition as described herein. In some embodiments, the therapeutic agent comprises a polyribonucleotide. In some embodiments, the polyribonucleotide comprises an mRNA coding for a therapeutic protein. In some embodiments, the therapeutic agent comprises a gene editing system. Delivery of the payload can be carried out by any of the means described herein, including administration of the filled TLA composition to a patient by any one or more routes of administration typically employed to deliver therapeutics and/or prophylactics to a subject.

In some aspects, the present disclosure provides the population of lipid assemblies or pharmaceutical composition described herein for use in specifically delivering a therapeutic and/or prophylactic agent to a targeted cell in a subject.

In some aspects, the present disclosure provides use of the TLA or TLNP compositions described herein in the manufacture of a medicament for delivering a therapeutic agent to a cell in a subject.

In some embodiments, the subject is human.

The present disclosure provides methods of delivering a therapeutic and/or prophylactic, such as a nucleic acid, to a mammalian cell or organ. Delivery of a therapeutic and/or prophylactic to a cell involves administering a formulation of the disclosure that comprises a TLA including the therapeutic and/or prophylactic, such as a nucleic acid, to a subject, where administration of the composition involves contacting the cell with the composition. In some embodiments, a protein, cytotoxic agent, radioactive ion, chemotherapeutic agent, or nucleic acid (such as an RNA, e.g., mRNA) may be delivered to a cell or organ. In the instance that a therapeutic and/or prophylactic is an mRNA, upon contacting a cell with the TLA, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a TLA may target a particular type or class of cells (e.g., cells of a particular organ or system thereof). In some embodiments, a TLA including a therapeutic and/or prophylactic of interest may be specifically delivered to a mammalian liver, kidney, spleen, femur, or lung. Specific delivery to a particular class of cells, an organ, or a system or group thereof implies that a higher proportion of lipid assemblies including a therapeutic and/or prophylactic are delivered to the destination (e.g., tissue) of interest relative to other destinations, e.g., upon administration of a TLA to a mammal. In some embodiments, specific delivery may result in a greater than 2 fold, 5 fold, 10 fold, 15 fold, or 20 fold increase in the amount of therapeutic and/or prophylactic per 1 g of tissue of the targeted destination (e.g., tissue of interest, such as a liver) as compared to another destination (e.g., the spleen). In some embodiments, the tissue of interest is selected from the group consisting of a liver, kidney, a lung, a spleen, a femur, vascular endothelium in vessels (e.g., intra-coronary or intra-femoral) or kidney, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in a TLA. An mRNA may additionally or instead be used to direct the synthesis and extracellular localization of lipids, carbohydrates, or other biological moieties. Alternatively, other therapeutics and/or prophylactics or elements (e.g., lipids or ligands) of a TLA may be selected based on their affinity for particular receptors (e.g., low density lipoprotein receptors) such that a TLA may more readily interact with a target cell population including the receptors. In some embodiments, ligands may include, but are not limited to, members of a specific binding pair, antibodies, monoclonal antibodies, Fv fragments, single chain Fv (scFv) fragments, Fab′ fragments, F(ab′)₂ fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell.

In some embodiments, a TLA may target hepatocytes. Apolipoproteins such as apolipoprotein E (apoE) have been shown to associate with neutral or near neutral lipid-containing lipid assemblies in the body, and are known to associate with receptors such as low-density lipoprotein receptors (LDLRs) found on the surface of hepatocytes. Thus, a TLA including a lipid component with a neutral or near neutral charge that is administered to a subject may acquire apoE in a subject's body and may subsequently deliver a therapeutic and/or prophylactic (e.g., an RNA) to hepatocytes including LDLRs in a targeted manner.

### Methods of Producing Polypeptides in Cells

Also provided herein are methods of producing a polypeptide of interest in a target cell within a subject, comprising administering to the subject any of the filled TLA compositions described herein. In some embodiments, the target cell is a mammalian cell.

The present disclosure provides methods of producing a polypeptide of interest in a mammalian cell. Methods of producing polypeptides involve contacting a cell with a formulation of the disclosure comprising a TLA including an mRNA encoding the polypeptide of interest. Upon contacting the cell with the TLA, the mRNA may be taken up and translated in the cell to produce the polypeptide of interest.

In general, the step of contacting a mammalian cell with a TLA including an mRNA encoding a polypeptide of interest may be performed in vivo, ex vivo, in culture, or in vitro. The amount of TLA contacted with a cell, and/or the amount of mRNA therein, may depend on the type of cell or tissue being contacted, the means of administration, the physiochemical characteristics of the TLA and the mRNA (e.g., size, charge, and chemical composition) therein, and other factors. In general, an effective amount of the TLA will allow for efficient polypeptide production in the cell. Metrics for efficiency may include polypeptide translation (indicated by polypeptide expression), level of mRNA degradation, and immune response indicators.

The step of contacting a TLA including an mRNA with a cell may involve or cause transfection. A phospholipid including in the lipid component of a TLA may facilitate transfection and/or increase transfection efficiency, for example, by interacting and/or fusing with a cellular or intracellular membrane. Transfection may allow for the translation of the mRNA within the cell.

In some embodiments, the lipid assemblies described herein may be used therapeutically. For example, an mRNA included in a TLA may encode a therapeutic polypeptide (e.g., in a translatable region) and produce the therapeutic polypeptide upon contacting and/or entry (e.g., transfection) into a cell. In other embodiments, an mRNA included in a TLA may encode a polypeptide that may improve or increase the immunity of a subject. In some embodiments, an mRNA may encode a granulocyte-colony stimulating factor or trastuzumab.

In some embodiments, an mRNA included in a TLA may encode a recombinant polypeptide that may replace one or more polypeptides that may be substantially absent in a cell contacted with the TLA. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some embodiments, contacting a cell with a TLA including an mRNA may reduce the innate immune response of a cell to an exogenous nucleic acid. A cell may be contacted with a first TLA including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

### Methods of Treating Diseases and Disorders

In some aspects, the present disclosure provides a method of treating or preventing a disease or disorder, the method comprising administering to a subject in need thereof the TLA, TLNP or pharmaceutical composition described herein (e.g., in a therapeutically effective amount).

In some aspects, the present disclosure provides the TLA, TLNP or pharmaceutical composition described herein for use in treating or preventing a disease or disorder in a subject.

In some aspects, the present disclosure provides use of the TLA, TLNP or pharmaceutical composition described herein in the manufacture of a medicament for treating or preventing a disease or disorder.

In some embodiments, the disease or disorder is a liver disease or liver disorder. For example, in some embodiments, the disease or disorder is hemophilia A, hemophilia B, Wilson disease, transthyretin amyloidosis (TTR), hereditary angioedema (HAE), familial hyperlipidemias/hypercholesterolemias/hypertriglyceridemia, or homocystinuria. In some embodiments, the disease or disorder is PA (propionic acidemia), MMA (methylmalonic acidemia), GSD1a (glycogen storage disease type I), PKU (phenylketonuria), or OTC (ornithine transcarbamylase deficiency). In some embodiments, the disease or disorder is galactosemia, MSUD (maple syrup urine disease), UCDs (urea cycle disorders such as urea cycle disorders associated with ASL or ASS1 activity), GSD1b (glycogen storage disease type 1B), or PFICs (progressive familial intrahepatic cholestasis, such as PFIC type 1, PFIC type 2, or PFIC type 3). In some embodiments, the disease or disorder is associated with the activity of one or more of PCSK9, ANGPTL3, LP(a), and APOC3. In some embodiments, the disease or disorder is acute hepatic porphyrias (e.g., acute intermittent porphyria). In some embodiments, the disease or disorder is primary hyperoxaluria (PH1).

In some embodiments, the TLA, TLNP or pharmaceutical composition is administered parenterally.

In some embodiments, the TLA, TLNP or pharmaceutical composition is administered intramuscularly, intradermally, subcutaneously, and/or intravenously.

TLA and TLNP compositions may be useful for treating a disease, disorder, or condition. In particular, such compositions may be useful in treating a disease, disorder, or condition characterized by missing or aberrant protein or polypeptide activity. In some embodiments, a formulation of the disclosure that comprises a TLA/TLNP including an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment of acute diseases, disorders, or conditions such as sepsis, stroke, and myocardial infarction. A therapeutic and/or prophylactic included in a TLA may also be capable of altering the rate of transcription of a given species, thereby affecting gene expression.

The disclosure provides methods involving administering TLA, TLNP or pharmaceutical composition thereof including one or more therapeutic and/or prophylactic agents, such as a nucleic acid, and pharmaceutical compositions including the same. The terms therapeutic and prophylactic can be used interchangeably herein with respect to features and embodiments of the present disclosure. Therapeutic compositions, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more therapeutics and/or prophylactics employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A TLA or TLNP including one or more therapeutics and/or prophylactics, such as a nucleic acid, may be administered by any route. In some embodiments, compositions, including prophylactic, diagnostic, or imaging compositions including one or more lipid assemblies described herein, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, topical (e.g., by powders, ointments, creams, gels, lotions, and/or drops), mucosal, nasal, buccal, enteral, intravitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery or administration of compositions described herein by any appropriate route taking into consideration likely advances in the sciences of drug delivery. In general, the most appropriate route of administration will depend upon a variety of factors including the nature of the TLA including one or more therapeutics and/or prophylactics (e.g., its stability in various bodily environments such as the bloodstream and gastrointestinal tract), the condition of the patient (e.g., whether the patient is able to tolerate particular routes of administration), etc.

A TLA or TLNP including one or more therapeutics and/or prophylactics, such as a nucleic acid, may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. In some embodiments, one or more TLAs or TLNPs including one or more different therapeutics and/or prophylactics may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

A TLA or TLNP may be used in combination with an agent to increase the effectiveness and/or therapeutic window of the composition. Such an agent may be, for example, an anti-inflammatory compound, a steroid (e.g., a corticosteroid), a statin, an estradiol, a BTK inhibitor, an S1P1 agonist, a glucocorticoid receptor modulator (GRM), or an anti-histamine. In some embodiments, a TLA may be used in combination with dexamethasone, methotrexate, acetaminophen, an H1 receptor blocker, or an H2 receptor blocker. In some embodiments, a method of treating a subject in need thereof or of delivering a therapeutic and/or prophylactic to a subject (e.g., a mammal) may involve pre-treating the subject with one or more agents prior to administering a TLA. In some embodiments, a subject may be pre-treated with a useful amount (e.g., 10 mg, 20 mg, 30 mg, 40 mg, 50 mg, 60 mg, 70 mg, 80 mg, 90 mg, 100 mg, or any other useful amount) of dexamethasone, methotrexate, acetaminophen, an H1 receptor blocker, or an H2 receptor blocker. Pre-treatment may occur 24 or fewer hours (e.g., 24 hours, 20 hours, 16 hours, 12 hours, 8 hours, 4 hours, 2 hours, 1 hour, 50 minutes, 40 minutes, 30 minutes, 20 minutes, or 10 minutes) before administration of the TLA and may occur one, two, or more times in, for example, increasing dosage amounts.

### Combination Therapy

The TLA and TLNP compositions filled with one or more payload therapeutics and/or prophylactics, such as a nucleic acid, may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Lipid nanoparticle compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually. The particular combination of therapies (therapeutics or procedures) to employ in a combination regimen will take into account compatibility of the desired therapeutics and/or procedures and the desired therapeutic effect to be achieved.

A filled lipid nanoparticle composition may be used in combination with an agent to increase the effectiveness and/or therapeutic window of the composition. Such an agent may be, for example, an antiinflammatory compound, a steroid (e.g., a corticosteroid), a statin, an estradiol, a BTK inhibitor, an S1P1 agonist, a glucocorticoid receptor modulator (GRM), or an anti-histamine. In some embodiments, the lipid nanoparticle composition may be used in combination with dexamethasone, methotrexate, acetaminophen, an H1 receptor blocker, or an H2 receptor blocker. In some embodiments, a subject may be pre-treated with one or more agents prior to administering lipid nanoparticle composition.

### Kits and Devices

The present disclosure provides kits for conveniently and/or effectively using the lipid nanoparticle compositions (or TLA) of the present disclosure. Typically kits will comprise sufficient amounts and/or numbers of components to allow a user to perform multiple administrations to one or more subjects and/or to perform multiple experiments.

In one aspect, the present disclosure provides kits comprising the lipid nanoparticles of the present disclosure.

The kit can further comprise packaging and instructions and/or a delivery agent to form a formulation composition. The delivery agent can comprise a saline, a buffered solution, or a lipidoid.

In some aspects, the kit can include an empty lipid nanoparticle composition (or empty TLA) and a nucleic acid solution. In some aspects, the kit comprises a first container comprising an empty lipid nanoparticle composition, and a second container comprising a solution having a therapeutic or prophylactic agent. In some aspects, the kit further comprises instructions for combining (e.g., mixing) the content of the first container and the second container. In some embodiments the container can comprise a polytetrafluoroethylene (PTFE) bag.

### Definitions

In order that the present disclosure can be more readily understood, certain terms are first defined. As used in this application, except as otherwise expressly provided herein, each of the following terms shall have the meaning set forth below. Additional definitions are set forth throughout the application.

The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the present disclosure. Where a value is explicitly recited, it is to be understood that values which are about the same quantity or amount as the recited value are also within the scope of the present disclosure. Where a combination is disclosed, each subcombination of the elements of that combination is also specifically disclosed and is within the scope of the present disclosure. Conversely, where different elements or groups of elements are individually disclosed, combinations thereof are also disclosed. Where any element of an present disclosure is disclosed as having a plurality of alternatives, examples of that present disclosure in which each alternative is excluded singly or in any combination with the other alternatives are also hereby disclosed; more than one element of an present disclosure can have such exclusions, and all combinations of elements having such exclusions are hereby disclosed.

The term “about” as used in connection with a numerical value throughout the specification and the claims refers to an interval of accuracy, familiar and acceptable to a person skilled in the art such as, for example, an interval of accuracy of ±10%, unless otherwise specified.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

As used herein, the term “administered in combination” or “combined administration” or “combination therapy” means that two or more agents are administered to a subject at the same time or within an interval such that there can be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “delivering” means providing an entity to a destination. For example, delivering a polynucleotide to a subject can involve administering a nanoparticle composition including the polynucleotide to the subject (e.g., by an intravenous, intramuscular, intradermal, or subcutaneous route). Administration of a nanoparticle composition to a mammal or mammalian cell can involve contacting one or more cells with the lipid nanoparticle composition.

As used herein, “delivery agent” refers to any substance that facilitates, at least in part, the in vivo, in vitro, or ex vivo delivery of a polynucleotide to targeted cells.

As used herein, the term “effective amount” of an agent is that amount sufficient to effect beneficial or desired results, and, as such, an “effective amount” depends upon the context in which it is being applied. For example, in the context of administering an agent that increases protein expression in a target tissue, an effective amount of an agent is, for example, an amount of mRNA expressing sufficient amount of said protein to increase protein expression in the target tissue, as compared to the protein expression observed without administration of the agent. The term “effective amount” can be used interchangeably with “effective dose.”

As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

As used herein, the term “lipid assembly” or “lipid assemblies”, refers to a composition having a structure by the assembly of one or more lipids. The assembled one or more lipids may form a lipid single later, a lipid bilayer, or a combination thereof. In some embodiments, the lipid assembly comprises a lipid nanoparticle, a liposome, or a combination thereof. In some embodiments, the lipid assembly has a size of about 500 nm or less, about 450 nm or less, about 400 nm or less, about 350 nm or less, about 300 nm or less, about 250 nm or less, about 200 nm or less, about 150 nm or less, or about 100 nm or less. In some embodiments, the lipid assembly has a size ranging from about 1 nm to about 100 nm. In some embodiments, about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, or about 95% or greater of the surface area of the lipid assemblies comprises a lipid bilayer. In some embodiments, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, or about 5% or less of the surface area of the lipid assemblies comprises a lipid bilayer.

As used herein, the term “lipid nanoparticle” or “LNP” refers to a nanoparticle comprising one or more lipids. In some embodiments, the LNP has a size of about 500 nm or less, about 450 nm or less, about 400 nm or less, about 350 nm or less, about 300 nm or less, about 250 nm or less, about 200 nm or less, about 150 nm or less, or about 100 nm or less. In some embodiments, the LNP has a size ranging from about 1 nm to about 100 nm.

As used herein, the term “liposome” refers to a composite having at least one lipid bilayer. In some embodiments, the liposome has a size of about 500 nm or less, about 450 nm or less, about 400 nm or less, about 350 nm or less, about 300 nm or less, about 250 nm or less, about 200 nm or less, about 150 nm or less, or about 100 nm or less. In some embodiments, the liposome has a size ranging from about 1 nm to about 100 nm.

As used herein, the term “total lipids” refers to the collection of ionizable lipids, structural lipids, and phospholipids, and PEG lipids (to the extent of their existence) in a given composition (e.g., a population of lipid assemblies). In some embodiments, when a population of lipid assemblies is free of PEG lipid, the total lipids in the population is the total amount of the ionizable lipid, the structural lipid, and the phospholipid in the population. In some embodiments, when a population of lipid assemblies comprises a PEG lipid, the total lipids in the population is the total amount of the ionizable lipid, the structural lipid, the phospholipid, and the PEG lipid in the population.

As used herein, the term “lipid amine” refers to a lipid molecule having one or more amine functional groups appended thereto. The amine functional group can include one or more primary (NH₂), secondary (NHR), or tertiary amine groups (NR₂), where R denotes a non-hydrogen group such as an alkyl group, carbocyclic group, heterocyclic group, or substituted derivatives of the same. The lipid amine include sterol amines, where the lipid portion of the molecule is a steroid, such as cholesterol or a related moiety.

As used herein, the phrase, “moiety cleavable under physiological conditions” refers to, for example, an ester, amide, carbonate, carbamate, or urea moiety.

As used herein, “patient” refers to a subject (e.g., a human subject) who seeks or is in need of treatment, requires treatment, is receiving treatment, will receive treatment, or a subject who is under care by a trained professional for a particular disease or condition. The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration.

The present disclosure also includes salts of the compounds described herein. As used herein, “salts” refers to derivatives of the disclosed compounds wherein the parent compound is modified by converting an existing acid or base moiety to its salt form (e.g., by reacting the free base group with a suitable organic acid). Examples of salts include, but are not limited to, mineral or organic acid salts of basic residues such as amines; alkali or organic salts of acidic residues such as carboxylic acids; and the like. In some embodiments, the salt is a pharmaceutically acceptable salt. Lists of pharmaceutically acceptable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient.

As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

The term “therapeutic agent” refers to an agent that, when administered to a subject, has a therapeutic, diagnostic, and/or prophylactic effect and/or elicits a desired biological and/or pharmacological effect. For example, in some embodiments, an mRNA encoding a polypeptide can be a therapeutic agent.

As used herein, the term “alkyl” or “alkyl group” means a linear or branched, saturated hydrocarbon including one to twenty carbon atoms (e.g., one, two, three, four, five, six, seven, eight, nine, ten, eleven, twelve, thirteen, fourteen, fifteen, sixteen, seventeen, eighteen, nineteen, twenty carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkylene” refers to a linking alkyl group.

As used herein, the term “alkenyl” or “alkenyl group” means a linear or branched hydrocarbon including two to twenty carbon atoms (e.g., two, three, four, five, six, seven, eight, nine, ten, eleven, twelve, thirteen, fourteen, fifteen, sixteen, seventeen, eighteen, nineteen, twenty carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. In some embodiments, C₁₈ alkenyl may include one or more double bonds, e.g., one, two, or three double bonds. A C₁₈ alkenyl group including two double bonds may be a linoleyl group. Unless otherwise specified, an alkenyl group described herein refers to both unsubstituted and substituted alkenyl groups.

As used herein, the term “carbocycle,” “carbocyclyl,” or “carbocyclic group” means an optionally substituted mono- or multi-cyclic system including one or more rings of carbon atoms, e.g., one, two, three, or four rings. Rings may be three, four, five, six, seven, eight, nine, ten, eleven, twelve, thirteen, fourteen, fifteen, sixteen, seventeen, eighteen, nineteen, or twenty membered rings. In some embodiments, the rings may be three, four, five, six, seven, eight membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2-dihydronaphthyl groups.

The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “alkynyl” or “alkynyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one triple bond.

As used herein, the term “carbocyclylene” refers to a linking carbocyclyl group.

As used herein, the term “carbocyclylalkyl” refers to an alkyl group substituted by a carbocyclyl group. An example carbocyclylalkyl group is benzyl.

As used herein, the term “heteroalkyl” or “heteroalkyl group” means an optionally substituted alkyl group in which one, two, three, four, five, six, or seven of the alkyl carbons has been replaced by one or more heteroatoms selected from O, S, or N, provided that not more than two consecutive alkyl carbons are replaced. A heteroalkyl group may be linear or branched. Examples of heteroalkyl groups include CH₂CH₂OCH₃, OCH₂CH₂OCH₂CH₂CH₃, CH₂NHCH₃, CH₂CH₂N(CH₃)CH₂CH₃, and CH₂CH₂OCH₃.

As used herein, the term “heteroalkylene” refers to a linking heteroalkyl group.

As used herein, the term “heterocycle,” “heterocyclyl,” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. Unless otherwise specified, heterocycles described herein refers to both unsubstituted and substituted heterocycle groups, i.e., optionally substituted heterocycles.

As used herein, the term “heterocycloalkyl” refers to a non-aromatic heterocycle, and represents a subset of heterocycles. Example heterocycloalkyl groups include azetidinyl, pyrolidinyl, piperidinyl, morpholinyl, and the like.

As used herein, the term “heterocyclylalkyl” refers to an alkyl group substituted by a heterocyclyl group.

As used herein, the term “heterocyclylene” refers to a linking heterocyclyl group.

As used herein, an “aryl group” is a carbocyclic group including one or more carbocyclic aromatic rings. Examples of aryl groups include phenyl and naphthyl groups.

As used herein, the term “arylene” refers to a linking aryl group.

As used herein, a “heteroaryl group” is a heterocyclic group including one or more heterocyclic aromatic rings. Examples of heteroaryl groups include pyrrolyl, furyl, thiophenyl, imidazolyl, oxazolyl, and thiazolyl. Both aryl and heteroaryl groups can be optionally substituted.

As used herein, the term “heteroarylene” refers to a linking heteroaryl group.

As used herein, the term “oxygen protecting group” refers to an oxo substituent that can be selectively removed under certain conditions (e.g., acidic or basic conditions).

Example oxygen protecting groups can include optionally substituted alkyl, carbocyclyl, heterocyclyl, carbocyclylalkyl, and heterocyclylalkyl groups.

As used herein, the term “nitrogen protecting group” refers to a nitrogen substituent (e.g., an amino substituent) that can be selectively removed under certain conditions (e.g., acidic of basic conditions). In some embodiments, the nitrogen protecting group is 9-fluorenylmethoxycarbonyl (Fmoc) or tert-butyloxycarbonyl (Boc).

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR are alkoxy groups that can be the same or different and R″″ is an alkyl or alkenyl group), a phosphate (e.g., P(O)₄³⁻), a thiol (e.g., —SH), a sulfoxide (e.g.,

—S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂, or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. In some embodiments, a C₁₋₆ alkyl group may be further substituted with one, two, three, four, five, or six substituents as described herein.

As used herein, the terms “approximately” and “about”, as applied to one or more values of interest, refer to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, when used in the context of an amount of a given compound in a lipid component of a lipid assembly, “about” may mean+/−10% of the recited value. For instance, a lipid assembly including a lipid component having about 40% of a given compound may include 30-50% of the compound.

As used herein, the term “compound,” is meant to include all isomers and isotopes of the structure depicted. “Isotopes” refers to atoms having the same atomic number but different mass numbers resulting from a different number of neutrons in the nuclei. In some embodiments, isotopes of hydrogen include tritium and deuterium. Further, a compound, salt, or complex of the present disclosure can be prepared in combination with solvent or water molecules to form solvates and hydrates by routine methods.

As used herein, the term “upon” intends to refer to the time point being after an action happens. For example, “upon administration” refers to the time point being after the action of administration.

As used herein, the term “contacting” means establishing a physical connection between two or more entities. In some embodiments, contacting a mammalian cell with a TLA means that the mammalian cell and a nanoparticle are made to share a physical connection. Methods of contacting cells with external entities both in vivo and ex vivo are well known in the biological arts. In some embodiments, contacting a TLA and a mammalian cell disposed within a mammal may be performed by varied routes of administration (e.g., intravenous, intramuscular, intradermal, and subcutaneous) and may involve varied amounts of lipid assemblies. Moreover, more than one mammalian cell may be contacted by a TLA.

As used herein, the term “comparable method” refers to a method with comparable parameters or steps, as of the method being compared (e.g., the producing the TLA formulation of the present disclosure). In some embodiments, the “comparable method” is a method with one or more of steps i), ia), iaa), ib), ii), iia), iib), iic), iid), and iie) of the method being compared. In some embodiments, the “comparable method” is a method without one or more of steps i), ia), iaa), ib), ii), iia), iib), iic), iid), and iie) of the method being compared. In some embodiments, the “comparable method” is a method without one or more of steps ia) and ib) of the method being compared. In some embodiments, the “comparable method” is a method employing a water-soluble salt of a nucleic acid. In some embodiments, the “comparable method” is a method employing an organic solution that does not comprise an organic solvent-soluble nucleic acid. In some embodiments, the “comparable method” is a method comprising processing the TLA prior to administering the TLA formulation.

As used herein, the term “delivering” means providing an entity to a destination. In some embodiments, delivering a therapeutic and/or prophylactic to a subject may involve administering a TLA including the therapeutic and/or prophylactic to the subject (e.g., by an intravenous, intramuscular, intradermal, or subcutaneous route). Administration of a TLA to a mammal or mammalian cell may involve contacting one or more cells with the TLA.

As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to the level of delivery of a therapeutic and/or prophylactic by a control nanoparticle to a target tissue of interest (e.g., MC3, KC2, or DLinDMA). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. It will be understood that the enhanced delivery of a nanoparticle to a target tissue need not be determined in a subject being treated, it may be determined in a surrogate such as an animal model (e.g., a rat model).

As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to an off-target tissue (e.g., mammalian spleen). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. In some embodiments, for renovascular targeting, a therapeutic and/or prophylactic is specifically provided to a mammalian kidney as compared to the liver and spleen if 1.5, 2-fold, 3-fold, 5-fold, 10-fold, 15 fold, or 20 fold more therapeutic and/or prophylactic per 1 g of tissue is delivered to a kidney compared to that delivered to the liver or spleen following systemic administration of the therapeutic and/or prophylactic. It will be understood that the ability of a nanoparticle to specifically deliver to a target tissue need not be determined in a subject being treated, it may be determined in a surrogate such as an animal model (e.g., a rat model).

As used herein, “encapsulation efficiency” refers to the amount of a therapeutic and/or prophylactic that becomes part of a TLA, relative to the initial total amount of therapeutic and/or prophylactic used in the preparation of a TLA. In some embodiments, if 97 mg of therapeutic and/or prophylactic are encapsulated in a TLA out of a total 100 mg of therapeutic and/or prophylactic initially provided to the composition, the encapsulation efficiency may be given as 97%.

As used herein, “encapsulation”, “encapsulated”, “loaded”, and “associated” may refer to complete, substantial, or partial enclosure, confinement, surrounding, or encasement. As used herein, “encapsulation” or “association” may refer to the process of confining an individual nucleic acid molecule within a nanoparticle and/or establishing a physiochemical relationship between an individual nucleic acid molecule and a nanoparticle. As used herein, an “empty nanoparticle” may refer to a nanoparticle that is substantially free of a therapeutic or prophylactic agent. As used herein, an “empty nanoparticle” may refer to a nanoparticle that is substantially free of a nucleic acid. As used herein, an “empty nanoparticle” may refer to a nanoparticle that consists substantially of only lipid components.

As used herein, “expression” of a nucleic acid sequence refers to translation of an mRNA into a polypeptide or protein and/or post-translational modification of a polypeptide or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

As used herein, a “lipid component” is that component of a TLA that includes one or more lipids. In some embodiments, the lipid component may include one or more cationic/ionizable, PEGylated, structural, or other lipids, such as phospholipids.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. In some embodiments, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. In some embodiments, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. In some embodiments, a modified nucleobase species may include one or more substitutions that are not naturally occurring.

As used herein, the “N:P ratio” is the molar ratio of ionizable (in the physiological pH range) nitrogen atoms in a lipid to phosphate groups in an RNA, e.g., in a TLA including a lipid component and an RNA.

As used herein, “naturally occurring” means existing in nature without artificial aid.

As used herein, “patient” refers to a subject who may seek or be in need of treatment, requires treatment, is receiving treatment, will receive treatment, or a subject who is under care by a trained professional for a particular disease or condition.

As used herein, a “PEG lipid” or “PEGylated lipid” refers to a lipid comprising a polyethylene glycol component.

As used herein, a “polymeric lipid” refers to a lipid comprising repeating subunits in its chemical structure. In some embodiments, the polymeric lipid is a lipid comprising a polymer component. In some embodiments, the polymeric lipid is a PEG lipid. In some embodiments, the polymeric lipid is not a PEG lipid. In some embodiments, the polymeric lipid is Brij or OH-PEG-stearate.

The phrase “pharmaceutically acceptable” is used herein to refer to those compounds, materials, composition, and/or dosage forms which are, within the scope of sound medical judgment, suitable for use in contact with the tissues of human beings and animals without excessive toxicity, irritation, allergic response, or other problems or complication, commensurate with a reasonable benefit/risk ratio.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In some embodiments, the nonaqueous media are ether, ethyl acetate, ethanol, isopropanol, or acetonitrile. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. In some embodiments, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the “polydispersity index” is a ratio that describes the homogeneity of the particle size distribution of a system. A small value, e.g., less than 0.3, indicates a narrow particle size distribution.

As used herein, an amphiphilic “polymer” is an amphiphilic compound that comprises an oligomer or a polymer. In some embodiments, an amphiphilic polymer can comprise an oligomer fragment, such as two or more PEG monomer units. In some embodiments, an amphiphilic polymer described herein can be PS 20.

As used herein, the term “polypeptide” or “polypeptide of interest” refers to a polymer of amino acid residues typically joined by peptide bonds that can be produced naturally (e.g., isolated or purified) or synthetically.

As used herein, an “RNA” refers to a ribonucleic acid that may be naturally or non-naturally occurring. In some embodiments, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. In some embodiments, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

As used herein, a “single unit dose” is a dose of any therapeutic administered in one dose/at one time/single route/single point of contact, i.e., single administration event.

As used herein, a “split dose” is the division of a single unit dose or total daily dose into two or more doses.

As used herein, a “total daily dose” is an amount given or prescribed in a 24 hour period. It may be administered as a single unit dose.

As used herein, the term “subject” refers to any organism to which a composition or formulation in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, “targeted cells” refers to any one or more cells of interest. The cells may be found in vitro, in vivo, in situ, or in the tissue or organ of an organism. The organism may be an animal. In some embodiments, the organism is a mammal. In some embodiments, the organism is a human. In some embodiments, the organism is a patient.

As used herein, “target tissue” refers to any one or more tissue types of interest in which the delivery of a therapeutic and/or prophylactic would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, the term “transfection” refers to the introduction of a species (e.g., an RNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. In some embodiments, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “zeta potential” refers to the electrokinetic potential of a lipid, e.g., in a particle composition.

As used herein, the term “polydispersity”, “polydispersity index”, or “PDI” refers to a measurement of the distribution of molecular mass in a given sample. The polydispersity is calculated as M_{w}/Mₘ in which M_{w} is the mass-average molar mass (or molecular weight) and Mₙ is the number-average molar mass (or molecular weight).

It is understood that some properties of lipid assemblies disclosed herein may be characterized by capillary zone electrophoresis (CZE). Capillary zone electrophoresis (CZE) refers to a separation technique which uses high voltage across a capillary to separate charged species based on their electrophoretic mobility. In some embodiments, the CZE is conducted with an acetate buffer (e.g., 50 mM sodium acetate at pH 5). In some embodiments, the CZE is conducted with a reverse voltage of about 10 kV across a 75 um capillary of 20 cm effective length. In some embodiments, the capillary is coated with polyethyleneimine.

The term “mobility peak”, as used herein, refers to a peak representing the distribution of a substance (e.g., a population of lipid assemblies) as measured by CZE. In some embodiments, the intensity of the mobility peak is detected by scattered light. It is understood that the intensity of the peak may indicate the amount of the portion of the substance at the position of the peak. In some embodiments, the position of the peak is calculated against a neutral reference standard (e.g., DMSO) being characterized by a mobility peak at 0, and a charged reference standard (e.g., benzylamine) being characterized by a mobility peak at 1.0. In some embodiments, a population of lipid assemblies may exhibit more than one peaks as measured by CZE, and unless indicated otherwise, the mobility peak refers to the peak having the greatest peak area among the more than one peaks.

The term “free of”, as used herein, means not comprising the referenced component. For example, when a population, solution, or formulation is described as being “free of PEG lipid”, the population, solution, or formulation does not comprise PEG lipid (e.g., does not comprise a PEG lipid described herein (e.g., does not comprise PEG-DMG)).

Those skilled in the art will recognize, or be able to ascertain using no more than routine experimentation, many equivalents to the specific embodiments in accordance with the present disclosure described herein. The scope of the present disclosure is not intended to be limited to the above Description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the present disclosure (e.g., any nucleic acid or protein encoded thereby; any method of production; any method of use; etc.) can be excluded from any one or more claims, for any reason, whether or not related to the existence of prior art.

It is further appreciated that certain features, which are, for clarity, described in the context of separate embodiments, can also be provided in combination in a single embodiment. Conversely, various features which are, for brevity, described in the context of a single embodiment, can also be provided separately or in any suitable subcombination.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

The following examples are put forth so as to provide those of ordinary skill in the art with a description of how the compositions and methods claimed herein can be performed, made, and evaluated, and are intended to be purely exemplary and are not intended to limit the scope of the present disclosure.

### Example 1. Synthesis of Exemplary Compounds

Materials and Analytical Techniques. All chemicals were purchased from Sigma Aldrich, Fisher Scientific and Combi-Blocks and were used without purification unless noted otherwise. NMR spectra were obtained on Bruker 300 Ultrashield™.

### Procedure

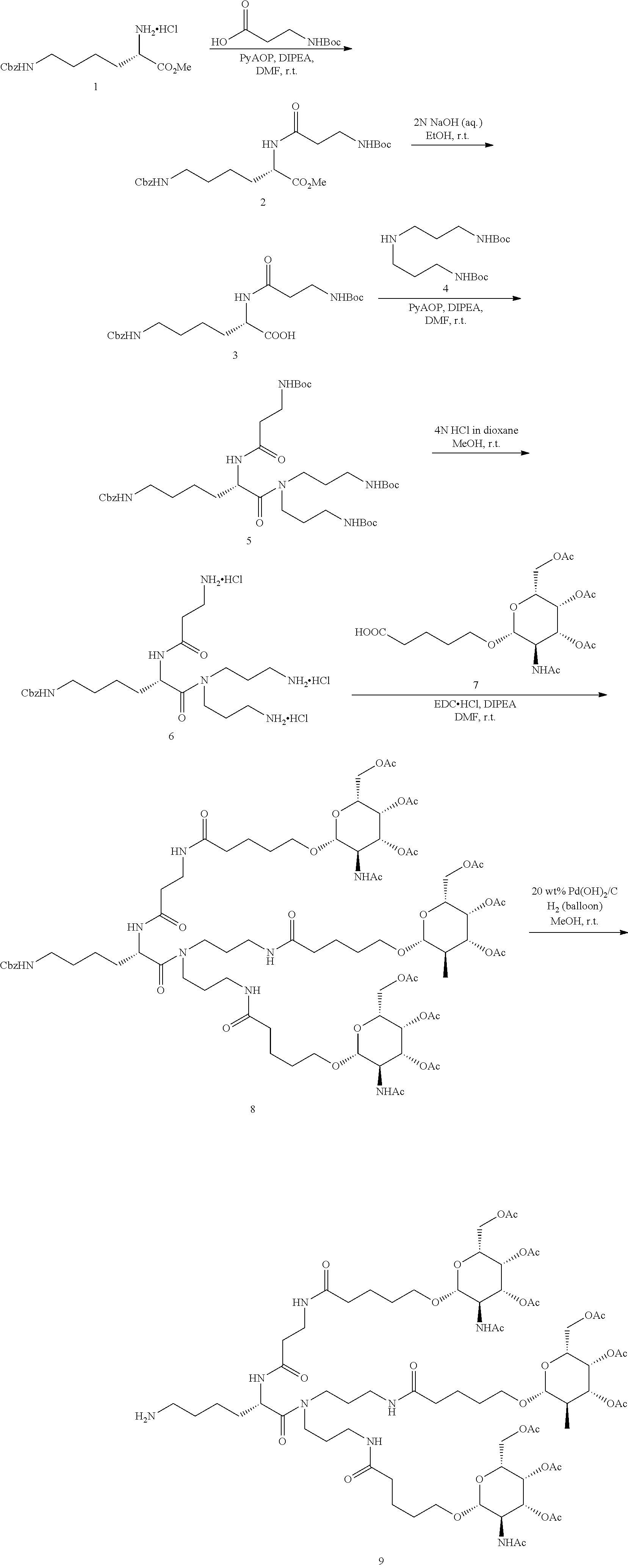

L-lysine methyl ester hydrochloride (1, 1 equiv.) and boc-beta-alanine (1.2 equiv.) were stirred with PyAOP (1.2 equiv.) in the presence of diisopropylethylamine (2.4 equiv) in DMF (0.3 M) at room temperature for overnight. The reaction mixture was poured into water and extracted with EtOAc. Combined organic layers were washed with water followed by brine, dried over MgSO₄, and concentrated under vacuo. Crude product was purified by column chromatography to give product 2 as a viscous oil.

Compound 2 (1 equiv.) was stirred in ethanol (0.4 M) and 2 N NaOH aqueous solution (0.4 M) at room temperature for overnight. Ethanol was removed under reduced pressure and resulting aqueous solution was acidified to pH 2-3 by adding 2 N HCl aqueous solution. Aqueous layer was extracted with DCM. Combined organic layers were dried over MgSO₄ and concentrated under vacuo to afford compound 3 as a viscous yellow oil. Crude reaction product was carried to the next step without purification.

Compound 3 (1 equiv.) and compound 4 (1.1 equiv.) were stirred with PyAOP (1.2 equiv.) in the presence of diisopropylethylamine (2.4 equiv.) in DMF (0.2 M) at room temperature for overnight. The reaction mixture was poured into water and extracted with EtOAc. Combined organic layers were washed with water followed by brine, dried over MgSO₄, and concentrated under vacuo. Crude product was purified by column chromatography to give product 5 as a clear viscous oil.

Compound 5 (1 equiv.) was stirred with 4N HCl solution in dioxane (6 equiv.) in MeOH (0.2 M) at room temperature for overnight. Solvent was removed under reduced pressure to give product 6 as a white powder. Crude reaction product was carried to the next step without purification.

Then, compound 6 (1 equiv.) and compound 7 (3 equiv.) were stirred with EDC-HCl (4 equiv.) in the presence of diisopropylethylamine (10 equiv.) in DCM (0.1 M) at room temperature for overnight. The reaction mixture was poured into water and extracted with DCM. Combined organic layers were dried over MgSO₄ and concentrated under vacuo. Crude product was purified by column chromatography to give product 8 as a clear viscous oil.

Then, compound 8 (1 equiv.) was stirred with 20 wt % palladium hydroxide on carbon (0.5 equiv.) in MeOH (0.05 M) at room temperature. Reaction was initially sparged with H₂ for 10 min and stirred for additional 1 h under H₂ atmosphere (balloon). The reaction mixture was filtered through a pad of celite and washed with MeOH. Solvent was removed under reduced pressure to give compound 9 as a clear viscous oil. Crude reaction product was carried to the next step without purification.

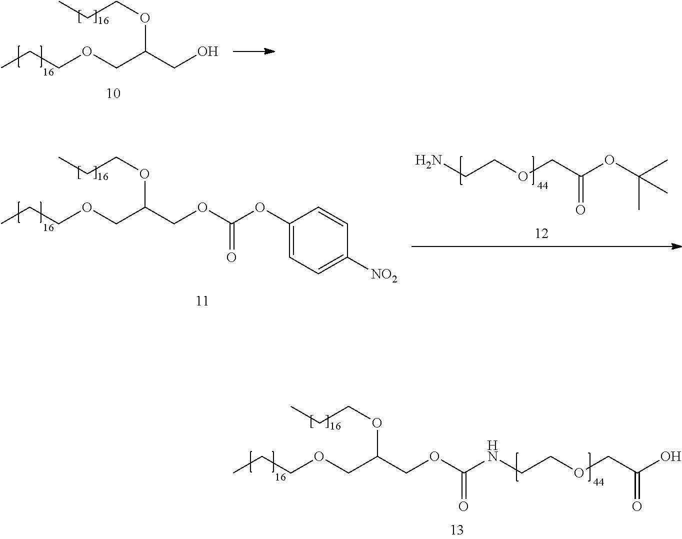

Compound 10 (1 equiv.) and 4-nitrophenyl chloroformate (2 equiv.) were stirred with diisopropylethylamine (2 equiv.) and 4-dimethylaminopyridine (0.1 equiv.) in THF (0.1 M) at room temperature for overnight. The reaction mixture was poured into saturated aqueous NaHCO₃ solution and extracted with diethyl ether. Combined organic layers were dried over MgSO₄ and concentrated under vacuo. Crude reaction mixture was purified by column chromatography to give compound 11 as a white solid.

H₂N-PEG2k-COOtBu (12, 1 equiv.) and compound 11 (1.2 equiv.) were stirred in DCM (0.08 M) in the presence of diisopropylethylamine (3 equiv.) at room temperature for overnight. Then, THF (0.24 M) was added to the reaction and stirred at room temperature for additional 6 h. Reaction was concentrated under reduced pressure. The reaction mixture was added into diethyl ether and stored at −20° C. for 30 min. Resulting white precipitate was collected by centrifugation and supernatant was discarded. Resulting solid was purified by preparative HPLC to give product 13 as a white solid.

### Synthesis of Compound 14

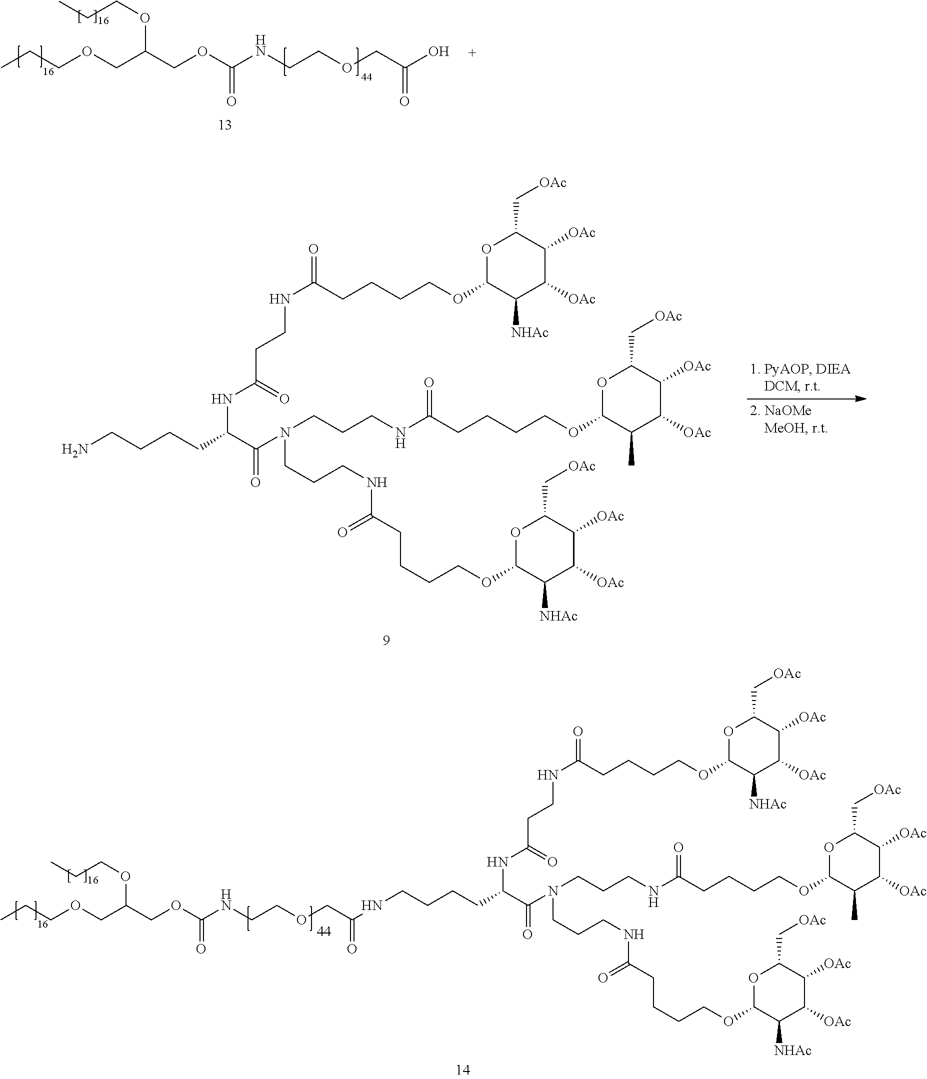

Compound 13 (1 equiv.) and compound 9 (1.5 equiv.) were stirred with PyAOP (1.5 equiv.) in the presence of diisopropylethylamine (5 equiv.) in DCM (0.02 M) at room temperature for overnight. Solvent was removed under reduced pressure and resulting solid was stirred with NaOMe (0.5 M in MeOH, 2 equiv.) in MeOH (0.02 M) at room temperature for overnight. The reaction mixture was purified by preparative HPLC to give 14 as a white solid.

Compound 14: ¹H NMR (300 MHz, CD₃OD) δ: ppm 4.36 (d, J=8.7 Hz, 3H), 3.97-3.73 (m, 15H), 3.64 (s, 116H), 3.60-3.38 (m, 15H), 2.52-2.42 (m, 6H), 2.28-2.14 (m, 6H), 1.98 (s, 9H), 1.75-1.48 (m, 28H), 1.30 (s, 60H), 0.97-0.83 (m, 6H).

### Synthesis of Compound 17

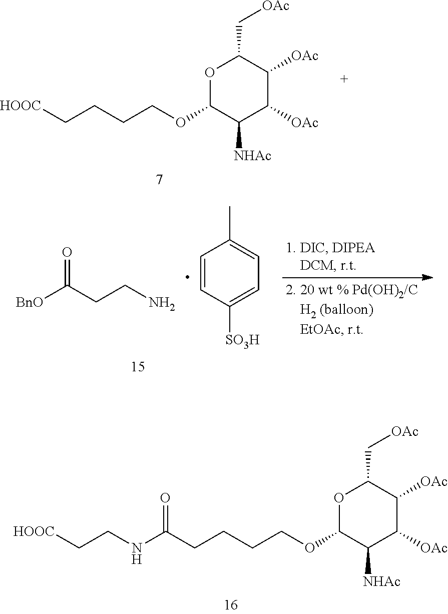 [SMILES: CC(=O)N[C@H]1[C@H](OCCCCC(=O)O)O[C@H](COC(C)=O)[C@H](OC(C)=O)[C@@H]1OC(C)=O ; NCCC(=O)OCc1ccccc1 ; Cc1ccc(S(=O)(=O)O)cc1 ; CC(=O)N[C@H]1[C@H](OCCCCC(=O)NCCC(=O)O)O[C@H](COC(C)=O)[C@H](OC(C)=O)[C@@H]1OC(C)=O]

Compound 7 (1 equiv.) and diisopropylcarbodiimide (1 equiv.) were stirred in the presence of diisopropylethylamine (2 equiv.) in DCM (0.2 M) at room temperature for 1 h. Then, compound 15 (1 equiv.) was added to the reaction and the reaction mixture was stirred at room temperature for overnight. The reaction mixture was poured into water and extracted with EtOAc. Combined organic layers were washed with brine, dried over MgSO₄, and concentrated under vacuo. Crude product was purified by column chromatography to give product as a clear viscous oil.

Then, the product was was stirred with 20 wt % palladium hydroxide on carbon (5 mol %) in EtOAc (0.1 M) at room temperature. Reaction was initially sparged with H₂ for 10 min and stirred for overnight under H₂ atmosphere (balloon). The reaction mixture was filtered through a pad of celite and washed with EtOAc. Solvent was removed under reduced pressure. Crude product was purified by column chromatography to give product 16 as a foamy solid.

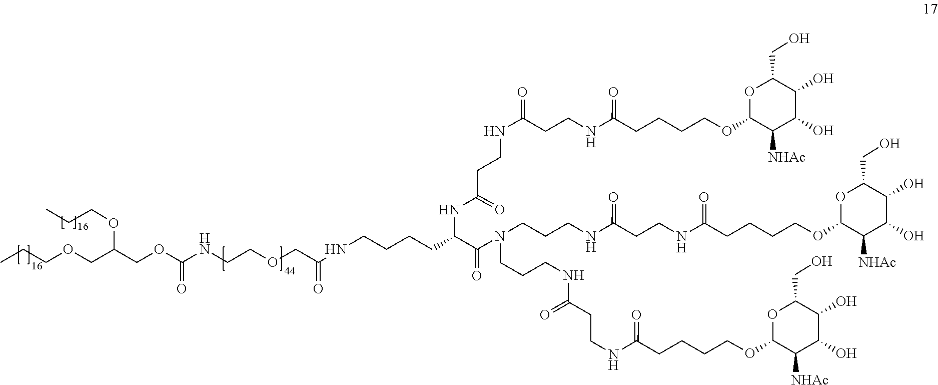

Compound 17 was synthesized by following the general procedure from the amide coupling between compound 6 and compound 16. Compound 17: ¹H NMR (300 MHz, CD₃OD) δ: ppm 4.38 (d, J=8.4 Hz, 3H), 4.10 (dt, J=11.4, 5.2 Hz, 3H), 3.99-3.74 (m, 15H), 3.66 (s, 149H), 3.58-3.38 (m, 26H), 2.54-2.36 (m, 12H), 2.22 (t, J=7.2 Hz, 6H), 2.01 (s, 9H), 1.92 (q, J=7.4 Hz, 3H), 1.81-1.50 (m, 22H), 1.32 (s, 60H), 0.97-0.88 (m, 6H).

### Synthesis of Compound 2

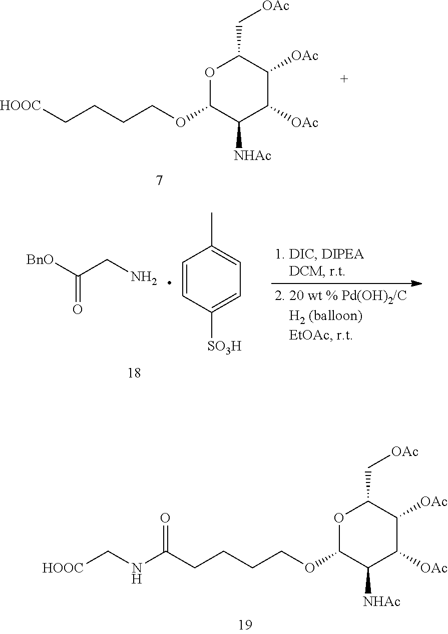 [SMILES: CC(=O)N[C@H]1[C@H](OCCCCC(=O)O)O[C@H](COC(C)=O)[C@H](OC(C)=O)[C@@H]1OC(C)=O ; NCC(=O)OCc1ccccc1 ; Cc1ccc(S(=O)(=O)O)cc1 ; CC(=O)N[C@H]1[C@H](OCCCCC(=O)NCC(=O)O)O[C@H](COC(C)=O)[C@H](OC(C)=O)[C@@H]1OC(C)=O]

Compound 7 (1 equiv.) and diisopropylcarbodiimide (1 equiv.) were stirred in the presence of diisopropylethylamine (2 equiv.) in DCM (0.2 M) at room temperature for 1 h. Then, compound 18 (1 equiv.) was added to the reaction and the reaction mixture was stirred at room temperature for overnight. The reaction mixture was poured into water and extracted with EtOAc. Combined organic layers were washed with brine, dried over MgSO₄, and concentrated under vacuo. Crude product was purified by column chromatography to give product as a clear viscous oil.

Then, the product was was stirred with 20 wt % palladium hydroxide on carbon (5 mol %) in EtOAc (0.1 M) at room temperature. Reaction was initially sparged with H₂ for 10 min and stirred for overnight under H₂ atmosphere (balloon). The reaction mixture was filtered through a pad of celite and washed with EtOAc. Solvent was removed under reduced pressure. Crude product was purified by column chromatography to give product 19 as a foamy solid.

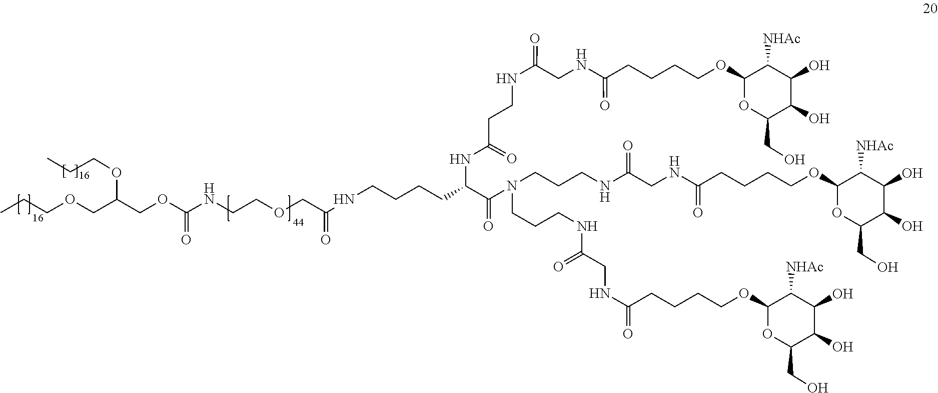

Compound 20 was synthesized by following the general procedure from the amide coupling between compound 6 and compound 19. Compound 20: ¹H NMR (300 MHz, CD₃OD) δ: ppm 4.36 (d, J=8.4 Hz, 3H), 4.09 (dt, J=11.5, 5.0 Hz, 3H), 3.98-3.69 (m, 22H), 3.64 (s, 130H), 3.59-3.37 (m, 24H), 2.50-2.26 (m, 12H), 1.99 (s, 9H), 1.93-1.49 (m, 29H), 1.30 (s, 60H), 0.95-0.86 (m, 6H).

### Synthesis of Compound 23

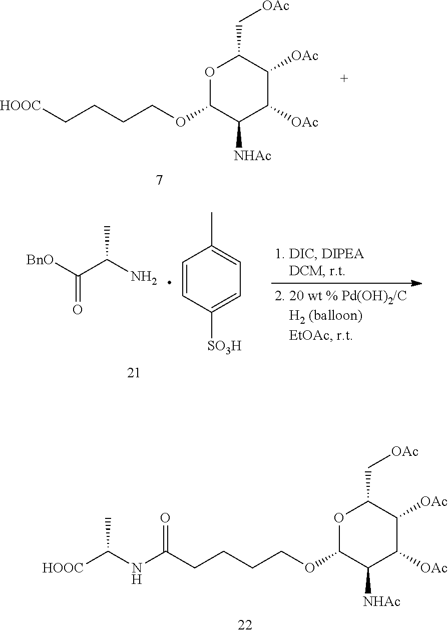 [SMILES: CC(=O)N[C@H]1[C@H](OCCCCC(=O)O)O[C@H](COC(C)=O)[C@H](OC(C)=O)[C@@H]1OC(C)=O ; C[C@H](N)C(=O)OCc1ccccc1 ; Cc1ccc(S(=O)(=O)O)cc1 ; CC(=O)N[C@H]1[C@H](OCCCCC(=O)N[C@@H](C)C(=O)O)O[C@H](COC(C)=O)[C@H](OC(C)=O)[C@@H]1OC(C)=O]

Compound 7 (1 equiv.) and diisopropylcarbodiimide (1 equiv.) were stirred in the presence of diisopropylethylamine (2 equiv.) in DCM (0.2 M) at room temperature for 1 h. Then, compound 21 (1 equiv.) was added to the reaction and the reaction mixture was stirred at room temperature for overnight. The reaction mixture was poured into water and extracted with EtOAc. Combined organic layers were washed with brine, dried over MgSO₄, and concentrated under vacuo. Crude product was purified by column chromatography to give product as a clear viscous oil.

Then, the product was was stirred with 20 wt % palladium hydroxide on carbon (5 mol %) in EtOAc (0.1 M) at room temperature. Reaction was initially sparged with H₂ for 10 min and stirred for overnight under H₂ atmosphere (balloon). The reaction mixture was filtered through a pad of celite and washed with EtOAc. Solvent was removed under reduced pressure. Crude product was purified by column chromatography to give product 22 as a foamy solid.

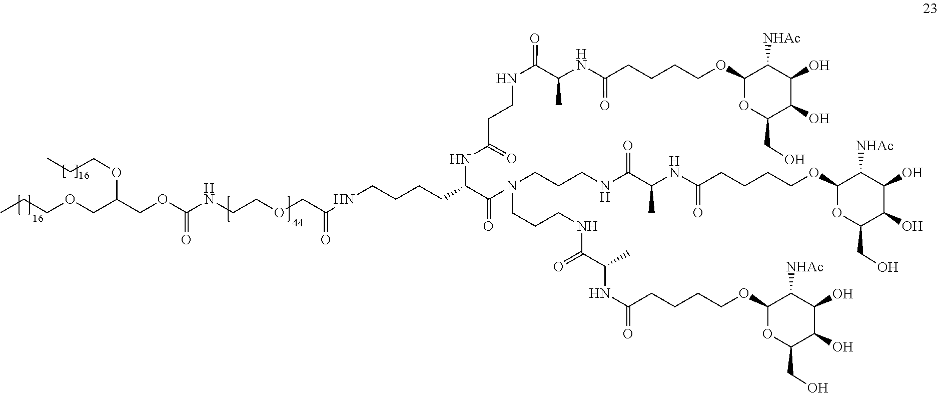

Compound 23 was synthesized by following the general procedure from the amide coupling between compound 6 and compound 22. Compound 23: ¹H NMR (300 MHz, CD₃OD) δ: ppm 4.38 (dd, J=8.4, 3.7 Hz, 3H), 4.31 (s, 3H), 4.15-3.71 (m, 19H), 3.66 (s, 126H), 3.60-3.36 (m, 23H), 2.50-2.25 (m, 12H), 2.01 (s, 9H), 1.94-1.49 (m, 28H), 1.32 (s, 60H), 0.97-0.86 (m, 6H).

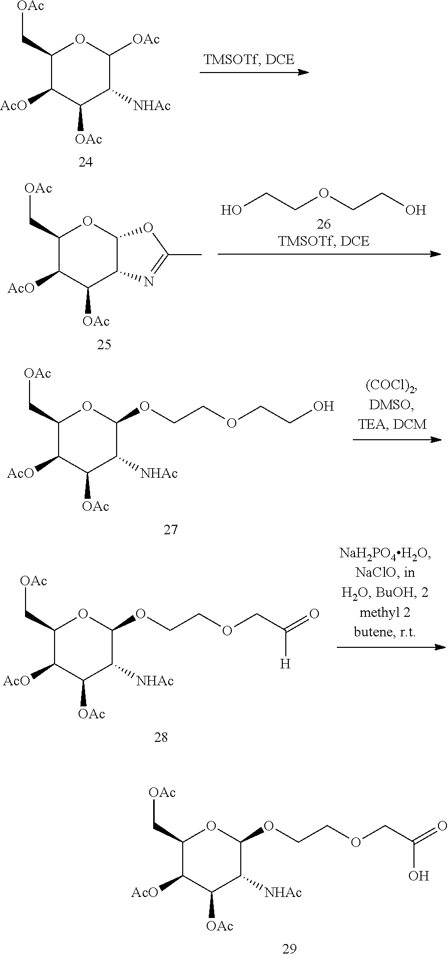 [SMILES: CC(=O)N[C@H]1C(OC(C)=O)O[C@H](COC(C)=O)[C@H](OC(C)=O)[C@@H]1OC(C)=O ; CC(=O)OC[C@H]1O[C@@H]2OC(C)=N[C@@H]2[C@@H](OC(C)=O)[C@H]1OC(C)=O ; OCCOCCO ; CC(=O)N[C@H]1[C@H](OCCOCCO)O[C@H](COC(C)=O)[C@H](OC(C)=O)[C@@H]1OC(C)=O ; CC(=O)N[C@H]1[C@H](OCCOCC=O)O[C@H](COC(C)=O)[C@H](OC(C)=O)[C@@H]1OC(C)=O ; CC(=O)N[C@H]1[C@H](OCCOCC(=O)O)O[C@H](COC(C)=O)[C@H](OC(C)=O)[C@@H]1OC(C)=O]

To a stirred solution of compound 24 (1 equiv.) in DCE (10 vol), TMSOTf (0.5 equiv.) was added at 0° C. and the reaction mixture was stirred at room temperature for 4 h. The reaction mixture was quenched with saturated aqueous NaHCO₃ solution and extracted with DCM. The organic layer was dried over Na₂SO₄ and concentrated under reduced pressure to afford compound 26 as a colorless oil.

To a stirred solution of 25 (1 equiv.) in DCE (10 vol), compound 26 (5 equiv.) and TMSOTf (0.5 equiv.) were added at 0° C. and stirred for 14 h at room temperature. The reaction mixture was washed with saturated aqueous NaHCO₃ and brine. The organic layer was dried over Na₂SO₄ and concentrated under reduced pressure. The crude residue was purified by column chromatography to afford compound 27 as a yellow oil.

To a stirred solution of DMSO (2.2 equiv.) and oxalyl Chloride (1.1 equiv.) in DCM (10 vol) at −40° C., a solution of compound 27 (1 equiv.) in DCM (10 vol) was added and stirred for 40 min at −40° C. Then triethylamine (10 equiv.) was added to the reaction mixture. The reaction mixture was quenched with saturated aqueous NaHCO₃ and brine. The organic layer was dried over Na₂SO₄ and concentrated under reduced pressure to afford 28 as a thick syrup.

To a stirred solution of 28 (1 equiv.) in in tert-butyl alcohol (20 vol) and 2-methyl-2-butene (5 vol), a solution of sodium chlorite (100 wt %) and sodium dihydrogen phosphate (100 wt %) in water (10 vol) was added dropwise over a 30 minute and the reaction mixture was stirred at room temperature for 12 h. Solvent was removed under reduced pressure and the residue was dissolved in water, acidified to pH 3 with 1 M HCl, and extracted with ethyl acetate. The organic layer was washed with brine, dried over Na₂SO₄ and concentrated under reduced pressure to afford compound 29 as a white solid.

Compound 29: ¹H NMR (400 MHz, DMSO-d₆) δ: ppm 12.5 (s, 1H), 7.81 (d, J=8.8 Hz, 1H), 5.21 (d, J=2 Hz, 1H), 4.98 (dd, J=3.6, 7.6 Hz, 1H), 4.86 (m, 1H), 4.08-3.95 (m, 4H), 3.93-3.70 (m, 2H), 3.65-3.40 (m, 4H), 2.10 (s, 3H), 2.0 (s, 3H), 1.89 (s, 3H), 1.77 (s, 3H). MS: 450.22 (M+H)⁺.

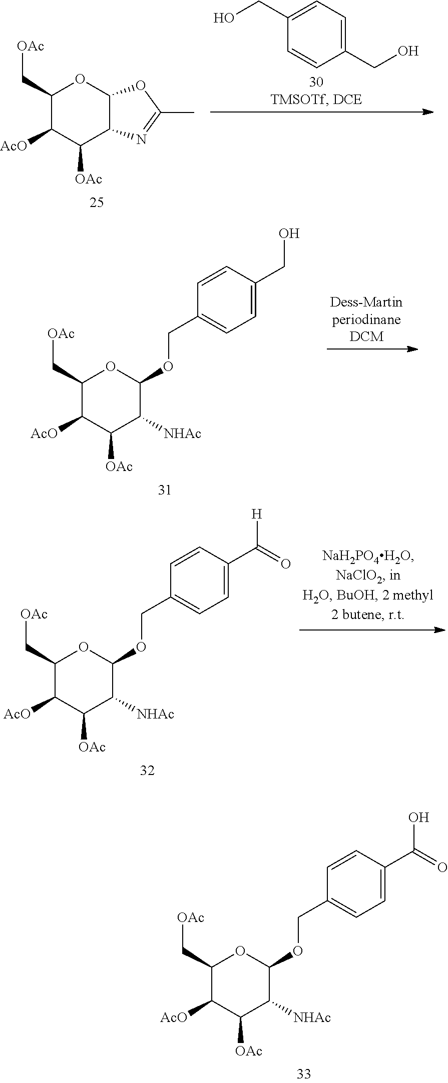 [SMILES: CC(=O)OC[C@H]1O[C@@H]2OC(C)=N[C@@H]2[C@@H](OC(C)=O)[C@H]1OC(C)=O ; OCc1ccc(CO)cc1 ; CC(=O)N[C@H]1[C@H](OCc2ccc(CO)cc2)O[C@H](COC(C)=O)[C@H](OC(C)=O)[C@@H]1OC(C)=O ; CC(=O)N[C@H]1[C@H](OCc2ccc(C=O)cc2)O[C@H](COC(C)=O)[C@H](OC(C)=O)[C@@H]1OC(C)=O ; CC(=O)N[C@H]1[C@H](OCc2ccc(C(=O)O)cc2)O[C@H](COC(C)=O)[C@H](OC(C)=O)[C@@H]1OC(C)=O]

To a stirred solution of 25 (1 equiv.) in dry DCE (10 vol) was added TMSOTf (0.5 equiv.) at 0° C. and stirred for 5 min. Compound 30 (2 equiv.) was added and the reaction mixture was stirred for 12 h at room temperature. The reaction mixture was diluted with DCM and washed with saturated aqueous NaHCO₃ solution. The organic layer was concentrated under reduced pressure and the crude was purified by silica gel column chromatography to afford compound 31 as a colorless oil.

To a stirred solution of 31 (1 equiv.) in DCM (10 vol), Dess-Martin periodinane (2 equiv.) was added and the suspension was stirred at room temperature for 3 h. The reaction mixture was filtered through celite and the filtrate was diluted with ethyl acetate, washed with saturated sodium thiosulfate solution, saturated NaHCO₃ solution and brine. The organic layer was dried over Na₂SO₄ and concentrate under reduced pressure to afford 32 as a colorless oil.

To a stirred solution of 32 (1 equiv.) in in tert-butyl alcohol (20 vol) and 2-methyl-2-butene (5 vol), a solution of sodium chlorite (100 wt %) and sodium dihydrogen phosphate (100 wt %) in water (10 vol) was added dropwise over a 30 minute and the reaction mixture was stirred at room temperature for 12 h. Solvent was removed under reduced pressure and the residue was dissolved in water, acidified to pH 3 with 1 M HCl, and extracted with ethyl acetate. The organic layer was washed with brine, dried over Na₂SO₄ and concentrated under reduced pressure to afford compound 33 as a white solid.

Compound 33: ¹H NMR (400 MHz, DMSO-d₆) δ: ppm 12.98 (s, 1H), 7.90 (t, J=7 Hz, 3H), 7.41 (d, J=7.6 Hz, 2H), 5.24 (d, J=3.2 Hz, 1H), 5.0 (dd, J=3.2, 11.2 Hz, 1H), 4.84 (d, J=13.2 Hz, 1H), 4.62 (d, J=10.8 Hz, 2H), 4.093-3.98 (m, 4H), 2.11 (s, 3H), 2.01 (s, 3H), 1.90 (s, 3H), 1.80 (s, 3H). MS: 482.25 (M+H)+.

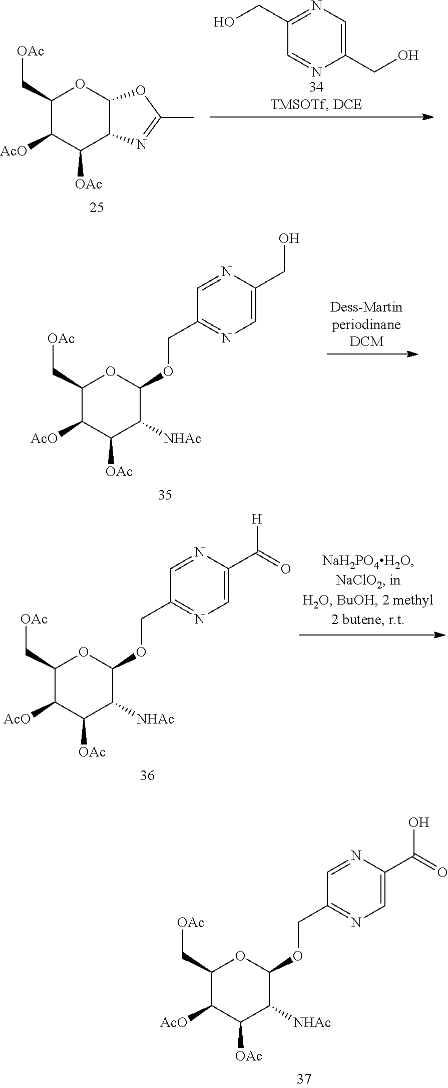 [SMILES: CC(=O)OC[C@H]1O[C@@H]2OC(C)=N[C@@H]2[C@@H](OC(C)=O)[C@H]1OC(C)=O ; OCc1cnc(CO)cn1 ; CC(=O)N[C@H]1[C@H](OCc2cnc(CO)cn2)O[C@H](COC(C)=O)[C@H](OC(C)=O)[C@@H]1OC(C)=O ; CC(=O)N[C@H]1[C@H](OCc2cnc(C=O)cn2)O[C@H](COC(C)=O)[C@H](OC(C)=O)[C@@H]1OC(C)=O ; CC(=O)N[C@H]1[C@H](OCc2cnc(C(=O)O)cn2)O[C@H](COC(C)=O)[C@H](OC(C)=O)[C@@H]1OC(C)=O]

To a stirred solution of 25 (1 equiv.) in dry DCE (10 vol) was added TMSOTf (0.5 equiv.) at 0° C. and stirred for 5 min. Compound 34 (2 equiv.) was added and the reaction mixture was stirred for 12 h at room temperature. The reaction mixture was diluted with DCM and washed with saturated aqueous NaHCO₃ solution. The organic layer was concentrated under reduced pressure and the crude was purified by silica gel column chromatography to afford compound 35 as a colorless oil.

To a stirred solution of 35 (1 equiv.) in DCM (10 vol), Dess-Martin periodinane (2 equiv.) was added and the suspension was stirred at room temperature for 3 h. The reaction mixture was filtered through celite and the filtrate was diluted with ethyl acetate, washed with saturated sodium thiosulfate solution, saturated NaHCO₃ solution and brine. The organic layer was dried over Na₂SO₄ and concentrate under reduced pressure to afford 36 as a colorless oil.

To a stirred solution of 36 (1 equiv.) in in tert-butyl alcohol (20 vol) and 2-methyl-2-butene (5 vol), a solution of sodium chlorite (100 wt %) and sodium dihydrogen phosphate (100 wt %) in water (10 vol) was added dropwise over a 30 minute and the reaction mixture was stirred at room temperature for 12 h. Solvent was removed under reduced pressure and the residue was dissolved in water, acidified to pH 3 with 1 M HCl, and extracted with ethyl acetate. The organic layer was washed with brine, dried over Na₂SO₄ and concentrated under reduced pressure to afford compound 37 as a white solid.

Compound 37: ¹H NMR (400 MHz, DMSO-d₆) δ: ppm 9.11 (s, 1H), 8.71 (s, 1H), 7.94 (d, J=8.8 Hz, 1H), 5.24 (s, 1H), 5.01-4.94 (m, 2H), 4.85-4.82 (m, 1H), 4.69 (d, J=8.4 Hz, 1H), 4.10-3.99 (m, 4H), 2.16 (s, 3H), 2.05 (s, 3H), 1.94 (s, 3H), 1.74 (s, 3H). MS: 484.40 (M+H)+.

Compounds 38, 39, and 40 were synthesized following the general procedure from compounds 29, 33, and 37.

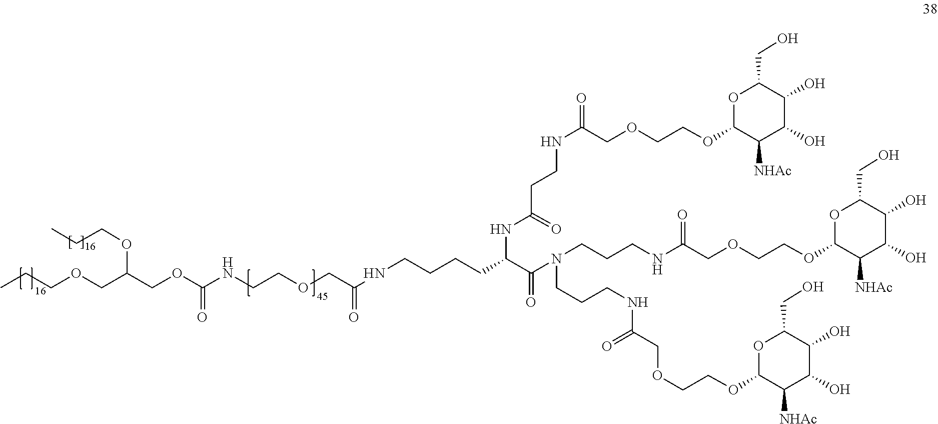

Compound 38: ¹H NMR (300 MHz, CD₃OD) δ: ppm 4.38 (d, J=8.5 Hz, 2H), 4.08-3.75 (m, 14H), 3.56 (s, 192H), 3.48-3.34 (m, 13H), 2.46 (t, J=6.9 Hz, 2H), 2.37 (t, J=6.3 Hz, 2H), 1.91 (d, J=3.4 Hz, 9H), 1.22 (s, 57H), 0.89-0.77 (m, 6H).

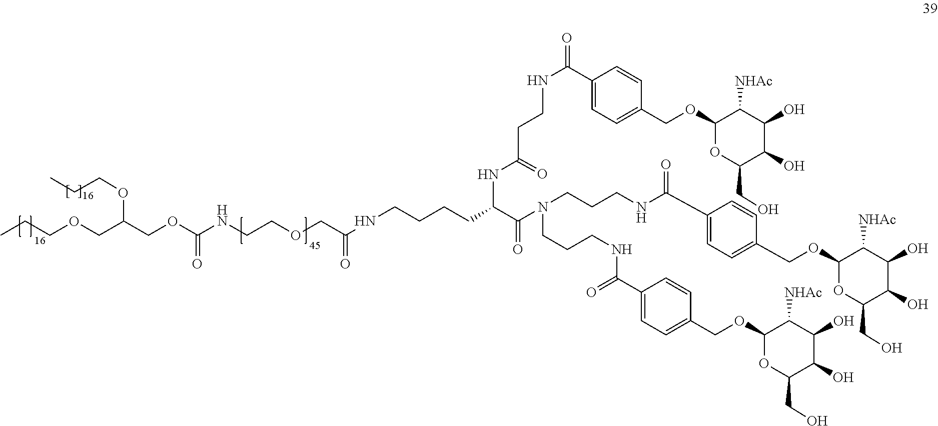

Compound 39: ¹H NMR (300 MHz, CD₃OD) δ: ppm 7.84-7.64 (m, 4H), 7.41 (t, J=9.0 Hz, 2H), 4.49 (t, J=6.8 Hz, 2H), 4.12-3.78 (i, 11H), 3.68 (s, 210H), 2.61 (s, 2H), 2.48-2.31 (m, 3H), 1.85 (d, J, 7.3 Hz, 2H), 1.58 (s, 5H), 1.31 (s, 57H), 1.00-0.82 (m, 6H).

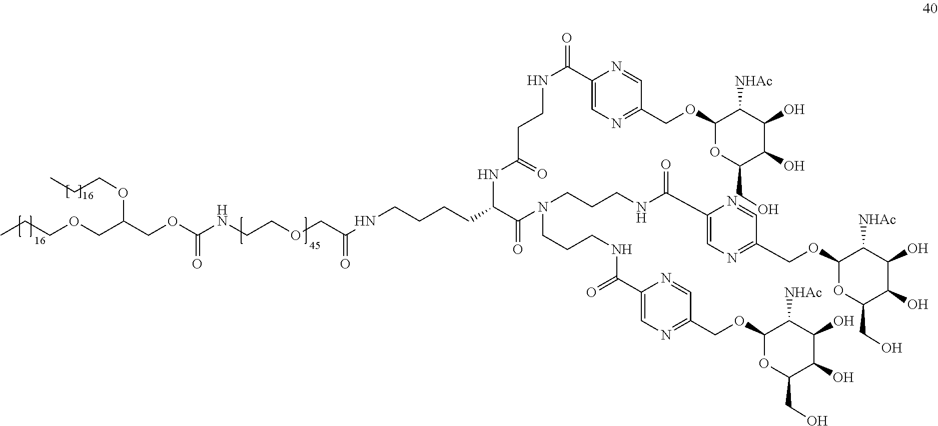

Compound 40: ¹H NMR (300 MHz, CD₃OD) δ: ppm 9.05-8.91 (m, 3H), 8.76-8.59 (m, 3H), 4.04-3.74 (m, 7H), 3.58 (s, 178H), 2.60-2.48 (m, 2H), 2.36 (t, J=6.2 Hz, 2H), 1.61 (d, J=79.0 Hz, 6H), 1.21 (s, 53H), 0.82 (t, J=6.4 Hz, 6H).

### Example 2. Formulation of the Lipid Nanoparticles

PEG targeting compounds were solubilized in water at a concentration of 10 mg/mL and then diluted to 1 mg/mL using 200 proof ethanol. Lipid stock solutions were prepared at a final concentration of 12.5 mM in 200 proof ethanol by combining the PEG targeting compound, ionizable lipid, phospholipid, structural lipid, and PEG lipid at a molar ratio of 0.5:48:11:38:1.5, or 48:11:38:2 for control samples with no PEG targeting compound. A solution of mRNA/sgRNA was diluted with 50 mM sodium acetate (pH 5) to a final concentration of 25 mM sodium acetate. A syringe pump fitted to a mixer was used to combine an aqueous solution of mRNA/sgRNA and the lipid stock solution at a flow rate ratio of 3:1 and N:P ratio of 4.9. After mixing, the product was transferred to Slide-A-Lyzer dialysis cassettes (Thermo Scientific, Rockford, IL, USA) with a molecular weight cutoff of 10 kDa. The cassettes were dialyzed for at least 18 hours against 20 mM tris/8% sucrose mM sodium chloride (pH 7.4) at a buffer volume at least 300 times greater than the product volume. The first dialysis was carried out at room temperature on a digital orbital shaker at 60-90 rpm for 3-4 h, then the buffer was replaced, and a second dialysis was carried out statically overnight at 4° C. Formulations were concentrated by Amicon centrifugal filters (MilliporeSigma, Burlington, MA, USA) with a molecular weight cutoff of 100 kDa, run through 0.22-μm syringe tip filters, and stored at 4° C. until use. Sample diameter and PDI were determined by dynamic light scattering, and nucleic acid concentration and encapsulation efficiency were determined by Ribogreen assay.

TABLE 1
Composition of targeted nanoparticles comprising lipids of the disclosure.

| | PEG | | | | | |
|---|---|---|---|---|---|---|
| TLNP (PEG | targeting | Ionizable | | Structural | | |
| targeting agent | agent | Lipid | Phospholipid | lipid | PEG lipid | |
| identity) | (% mol) | (% mol) | (% mol) | (% mol) | (% mol) | N:P |
| Base LNP | — | IL-1 (48) | DSPC (11.0) | Cholesterol | PGL-1 | 4.9 |
| (Control) | | | | (39) | (2.0) | |
| TLNP-14 | 0.5 | IL-1 (48) | DSPC (11.0) | Cholesterol | PGL-1 | 4.9 |
| | | | | (39) | (1.5) | |
| TLNP-17 | 0.5 | IL-1 (48) | DSPC (11.0) | Cholesterol | PGL-1 | 4.9 |
| | | | | (39) | (1.5) | |
| TLNP-20 | 0.5 | IL-1 (48) | DSPC (11.0) | Cholesterol | PGL-1 | 4.9 |
| | | | | (39) | (1.5) | |
| TLNP-23 | 0.5 | IL-1 (48) | DSPC (11.0) | Cholesterol | PGL-1 | 4.9 |
| | | | | (39) | (1.5) | |
| TLNP-38 | 0.5 | IL-1 (48) | DSPC (11.0) | Cholesterol | PL-1 | 4.9 |
| | | | | (39) | (1.5) | |
| TLNP-39 | 0.5 | IL-1 (48) | DSPC (11.0) | Cholesterol | PL-1 | 4.9 |
| | | | | (39) | (1.5) | |
| TLNP-40 | 0.5 | IL-1 (48) | DSPC (11.0) | Cholesterol | PL-1 | 4.9 |
| | | | | (39) | (1.5) | |

TABLE 2
Characteristics of nanoparticles comprising lipids of the disclosure.

| | | Diameter/Size | | % En- | |
|---|---|---|---|---|---|
| TLNP | Construct(s) | (nm) | PDI | capsulation | N:P |
| Base LNP | Cas9 + sgRNA | 72 | 0.17 | 94 | 4.9 |
| (Control) | | | | | |
| TLNP-14 | Cas9 + sgRNA | 78 | 0.22 | 93 | 4.9 |
| TLNP-17 | Cas9 + sgRNA | 76 | 0.17 | 93 | 4.9 |
| TLNP-20 | Cas9 + sgRNA | 69 | 0.14 | 93 | 4.9 |
| TLNP-23 | Cas9 + sgRNA | 75 | 0.18 | 89 | 4.9 |
| TLNP-38 | Cas9 + sgRNA | 59 | 0.19 | 98 | 4.9 |
| TLNP-39 | Cas9 + sgRNA | 61 | 0.23 | 98 | 4.9 |
| TLNP-40 | Cas9 + sgRNA | 57 | 0.20 | 99 | 4.9 |

### Example 3. Dosing and Analysis of Gene Editing Efficiency

### Dosing

To assess the effects of novel targeting ligands on tissue expression profile and expression strength of genome editors in vivo, female 9-11-week-old C57BL6/J (JAX #000664) mice as well as age- and sex-matched LDLR-ko mice (B6.129S7-LdlrtmlHer/J, JAX #002207) were used. Animals were purchased from Jackson labs at 7-8 weeks of age and allowed to acclimate for 2 weeks in Moderna's facilities. Routinely, mice were housed in groups 5 animals per cage. The mice were housed in a temperature- and humidity-controlled environment on a 12 hr-12 hr light-dark cycle with ad libitum access to water and diet (Prolab® Isopro® RMH 3000 5P76).

Mice were dosed intravenously with test articles at 0.2 mg/kg in a volume of 5 ml/kg. One week after dosing, the animals were sacrificed by CO2 asphyxiation followed by cervical dislocation. Blood was collected by cardiac puncture, and livers and spleens were harvested and snap frozen in liquid nitrogen for further analysis. Serum was prepared by incubating blood samples in serum separator tubes (BD) for >10 min, followed by centrifugation at 7000 g for 7 min. Serum was then snap frozen for further analysis.

All research involving animals was conducted in accordance with the Moderna Tx, Inc. Animal Care and Use guidelines and approved by the IACUC.

### Analysis of Gene Editing Efficiency

Mouse tissue from mice treated with TLNPs containing gene editing cargo or cells transfected with gene editing cargo were lysed and genomic DNA was extracted using Maxwell (Promega) or Quick Extract (Lucigen), respectively. Sequencing of the resulting genomic DNA was used to determine editing efficiencies by measuring insertions and deletions at the target site. Primers were designed flanking the target region to generate an amplicon. Additional PCR was performed to add adapters to allow for Illumina based sequencing on a MiSeq instrument. The resulting reads were aligned to a reference amplicon generated from a reference genome using CRISPResso 2.0 software.

Libraries which passed QC for read number, alignment, and substitution rate were evaluated for number of wild-type reads versus edited reads (i.e. read that contain an insertion or deletion). Editing percentage was evaluated by dividing the number of reads containing an insertion or deletion by the total number of reads, including edited plus wild-type reads. Results are shown in Table 3.

TABLE 3
Editing efficiency in liver following administration
of loaded TLNPs of the disclosure

% InDel

| TLNP | WT (wild-type) | KO (knockout) |
|---|---|---|
| Base | 3.4 | 3.4 |
| LNP (Control) | | |
| TLNP-14 | 35.0 | 55.6 |
| TLNP-17 | 46.1 | 42.9 |
| TLNP-20 | 39.2 | 38.0 |
| TLNP-23 | 33.8 | 39.2 |
| TLNP-38 | 19.2 | 28.4 |
| TLNP-39 | 15.3 | 14.8 |
| TLNP-40 | 9.4 | 12.9 |

What is claimed is:

1. A targeting compound of Formula I, a stereoisomer thereof, a tautomer thereof, and/or a pharmaceutically acceptable salt thereof: 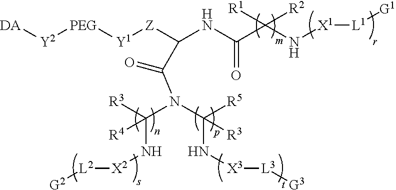
wherein,
DA is di(C₁₂₋₂₄alkanoyl)glycero or di(C₁₂₋₂₄alkyl)glycero;
G¹, G², and G³ are each independently selected from an asialoglycoprotein receptor targeting monosaccharide;
L¹, L², and L³ are, at each occurrence, independently selected from alkylene, alkenylene, heteroalkylene, cycloalkylene, heterocyclylene, arylene, or heteroarylene groups, or any combination of 2 or 3 of the foregoing groups;
PEG is a poly(ethylene glycol) having 1 to 100 ethylene oxy subunits;
X¹, X² and X³ are, at each occurrence, independently absent, C(O), C(O)O, or C(O)NH;
Y¹ is absent or a C₁₋₆ alkylene, O, C₁₋₆ alkylene-O, NH, C₁₋₆ alkylene-NH, C(O), C(O)NH, or C₁₋₆ alkylene-C(O)NH group;
Y² is absent or an O, C₁₋₆ alkylene-O, NH, C₁₋₆ alkylene-NH, C(O), C(O)O, or C(O)NH group;
Z is C₁₋₆ alkylene or 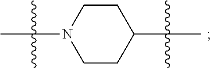 [SMILES: *C1CCN(*)CC1]
R¹, R², R³, R⁴, R⁵, and R⁶ at each occurrence are independently H or a C₁₋₆ alkyl group;
m is 1, 2, 3, 4, 5, or 6;
n, and p are each independently selected from 2, 3, 4, 5 or 6; and
r, s, and t are each independently 1, 2, 3, 4, or 5.

2. The targeting compound of claim 1 having a structure of Formula IA1 or Formula IA2:

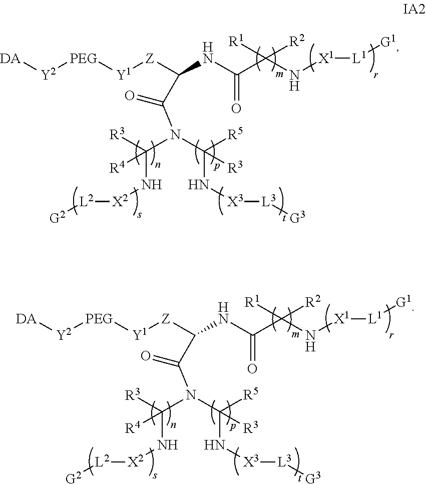

3. The targeting compound of claim 1, wherein:
R¹ and R² at each occurrence are H; or
R³ and R⁴ at each occurrence are H; or
R⁵ and R⁶ at each occurrence are H.

4. The targeting compound of claim 1, wherein at least one occurrence of one of R¹, R², R³, R⁴, R⁵, and R⁶ is not H.

5. The targeting compound of claim 1, wherein at least one occurrence of one of R¹, R², R³, R⁴, R⁵, and R⁶ is a methyl group.

6. The targeting compound of claim 1, having a structure of Formula IB:

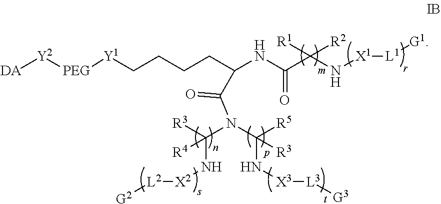

7. The targeting compound of claim 1, having a structure of any one of Formulae IC, ID, IE, IF, or IG:

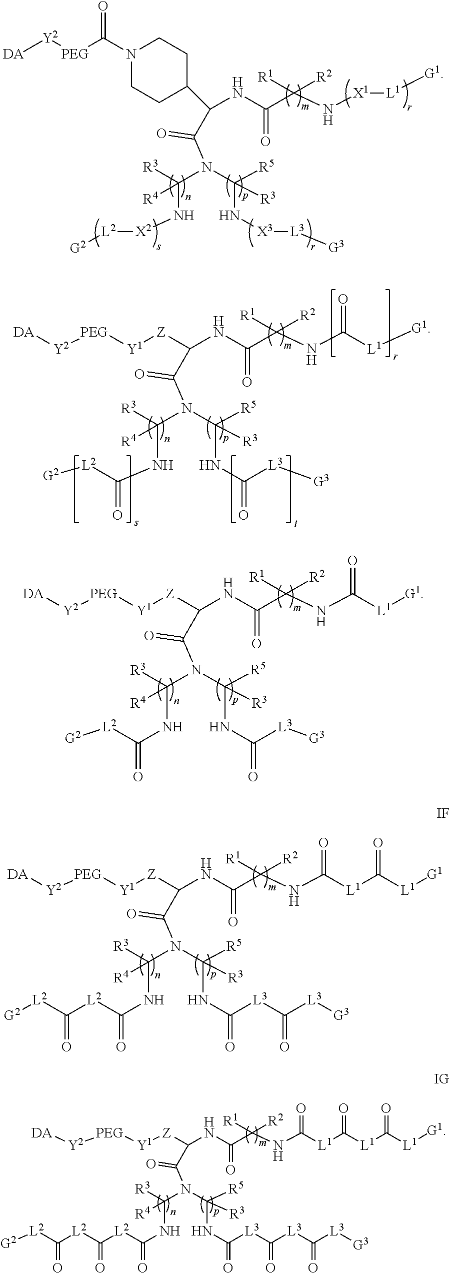

8. The targeting compound of claim 1, wherein G¹, G², and G³ each independently have the structure of Formula A or B: 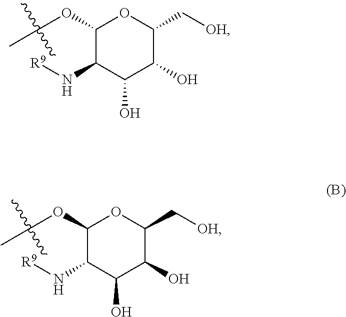
wherein
R⁹ is R¹⁰C(O), R¹⁰S(O)₂, or R¹⁰OC(O); and
R¹⁰ is an alkyl or alkenyl group.

9. The targeting compound of claim 1, wherein the targeting compound is selected from the group consisting of: 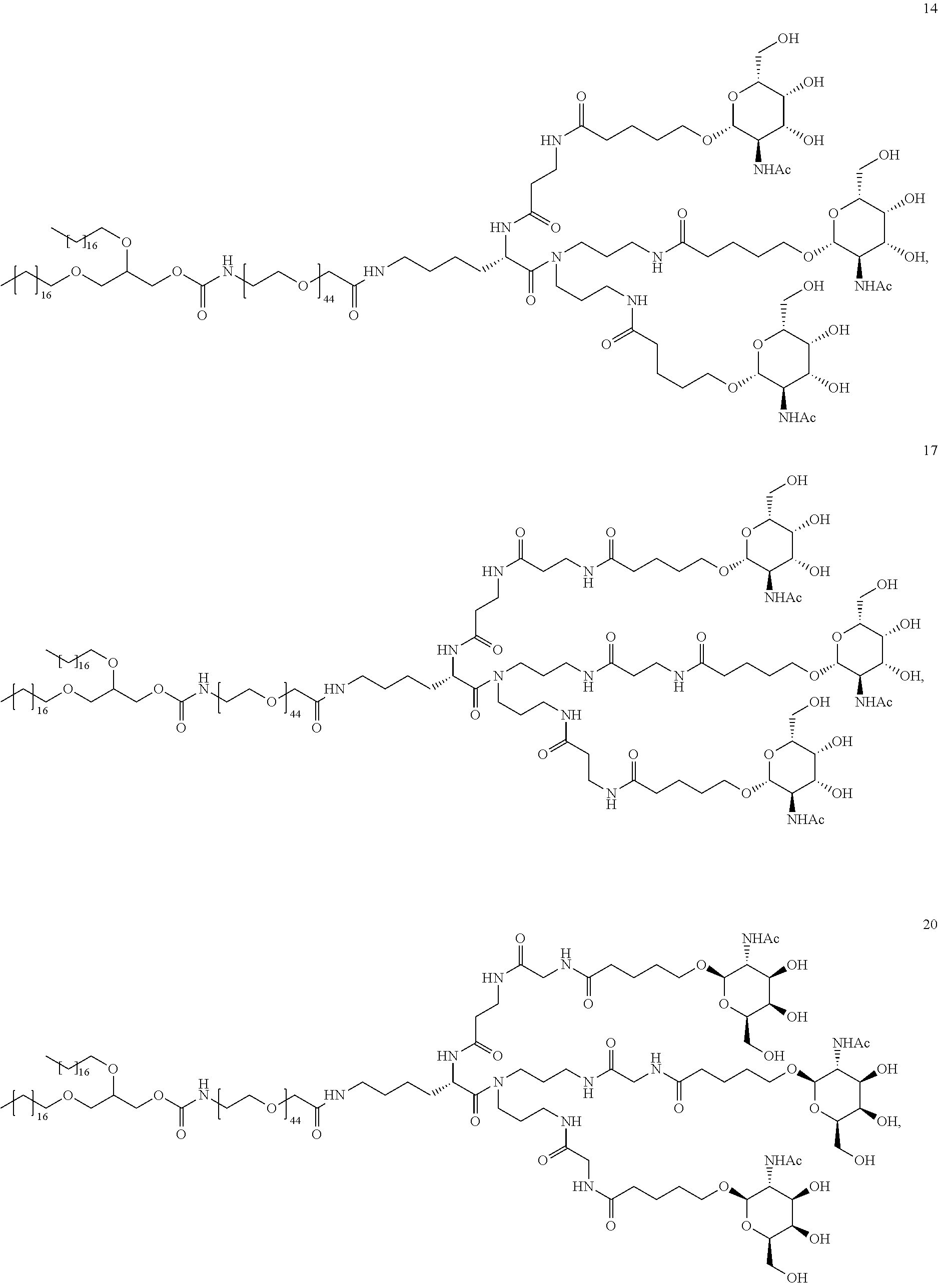 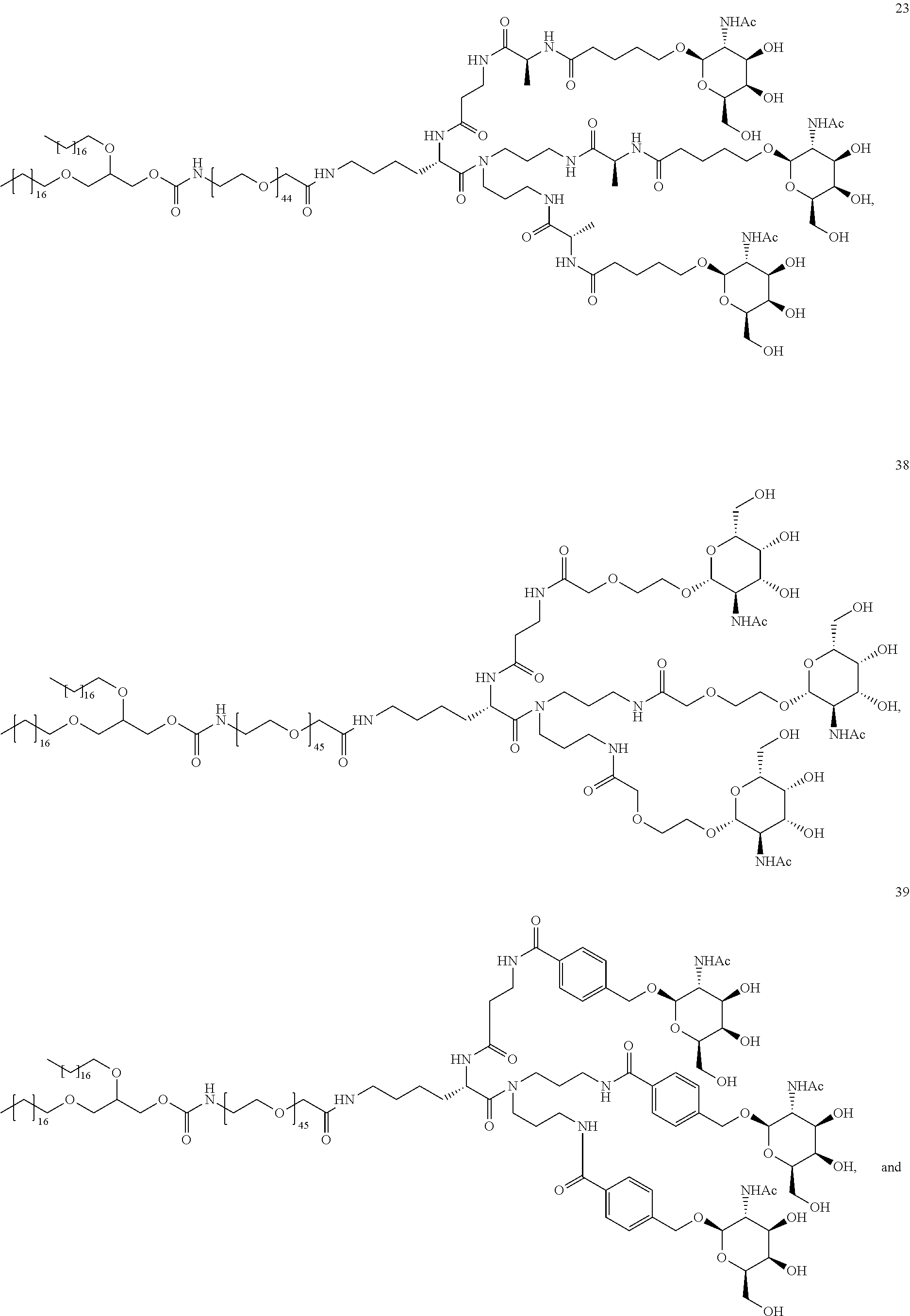 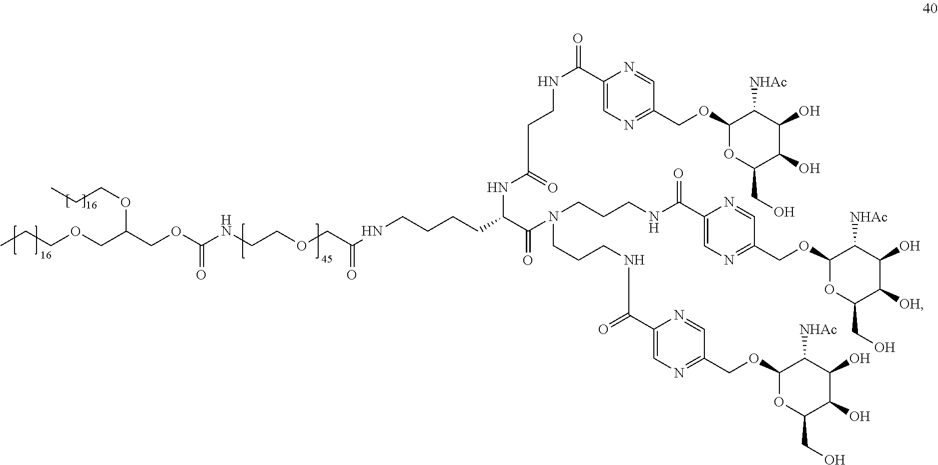
a stereoisomer thereof, a tautomer thereof, and/or a pharmaceutically acceptable salt thereof.

10. A targeted lipid assembly (TLA) or pharmaceutically acceptable salts thereof,
wherein the TLA comprises:
the targeting compound of claim 1;
an ionizable lipid;
a structural lipid;
a phospholipid; and
a PEG-lipid.

11. The TLA of claim 10, wherein the targeting compound is present at 0.025 to 10 mol %.

12. The TLA of claim 10, wherein the ionizable lipid comprises a compound of Formula IL, 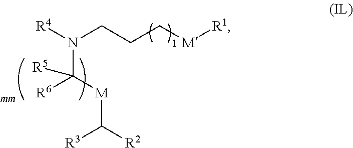
and pharmaceutically acceptable salts thereof, or an N-oxide or a salt thereof, wherein:
R²¹ is 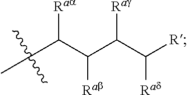
wherein 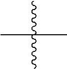
denotes a point of attachment;
R^{aα}, R^{aβ}, R^{aγ}, and R^{aδ} are each independently selected from H, C₂₋₁₂ alkyl, and C₂₋₁₂ alkenyl;
R²² and R²³ are each independently selected from C₁₋₁₄ alkyl and C₂₋₁₄ alkenyl;
R²⁴ is selected from —(CH₂)ₙOH and 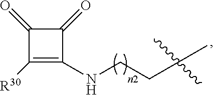
wherein n is selected from 1, 2, 3, 4, and 5;
wherein 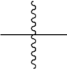
denotes a point of attachment,
wherein R³⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R²⁵ is independently selected from C₁₋₃ alkyl, C₂₋₃ alkenyl, and H;
each R²⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
ll is selected from 1, 2, 3, 4, and 5; and
mm is selected from 5, 6, 7, 8, 9, 10, 11, 12, and 13.

13. The TLA of claim 12, wherein
R²¹ is 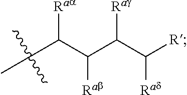
wherein 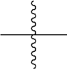
denotes a point of attachment;
R^{aα}, R^{aβ}, R^{aγ}, and R^{aδ} are each H;
R²¹ is 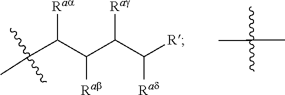
wherein 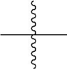
denotes a point of attachment;
R^{aα}, R^{aβ}, R^{aγ}, and R^{aδ} are each H;
R²² and R²³ are each C₁₋₁₄ alkyl;
R²⁴ is —(CH₂)ₙₙOH;
nn is 2;
each R²⁵ is H;
each R²⁶ is H;
M and M′ are each —C(O)O—;
R′ is C₁₋₁₂ alkyl;
ll is 5; and
mm is 7.

14. The TLA of claim 12, wherein
R²¹ is 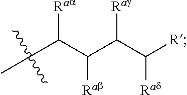
wherein 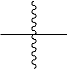
denotes a point of attachment;
R^{aα} is C₂₋₁₂ alkyl;
R^{aβ}, R^{aγ}, and R^{aδ} are each H;
R²² and R²³ are each C₁₋₁₄ alkyl;
R²⁴ is 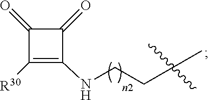
R³⁰ is —NH(C₁₋₆ alkyl);
n2 is 2;
each R²⁵ is H;
each R²⁶ is H;
M and M′ are each —C(O)O—;
R′ is C₁₋₁₂ alkyl;
ll is 5; and
mm is 7.

15. The TLA of claim 12, wherein
R²¹ is 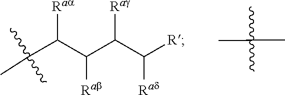
wherein 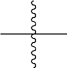
denotes a point of attachment;
R^{aα}, R^{aβ}, and R^{aδ} are each H;
R^{aγ} is C₂₋₁₂ alkyl;
R²² and R²³ are each C₁₋₁₄ alkyl;
R²⁴ is —(CH₂)ₙₙOH;
nn is 2;
each R²⁵ is H;
each R²⁶ is H;
M and M′ are each —C(O)O—;
R′ is C₁₋₁₂ alkyl;
ll is 5; and
mm is 7.

16. The TLA of claim 10, wherein the ionizable lipid is a compound selected from the group consisting of 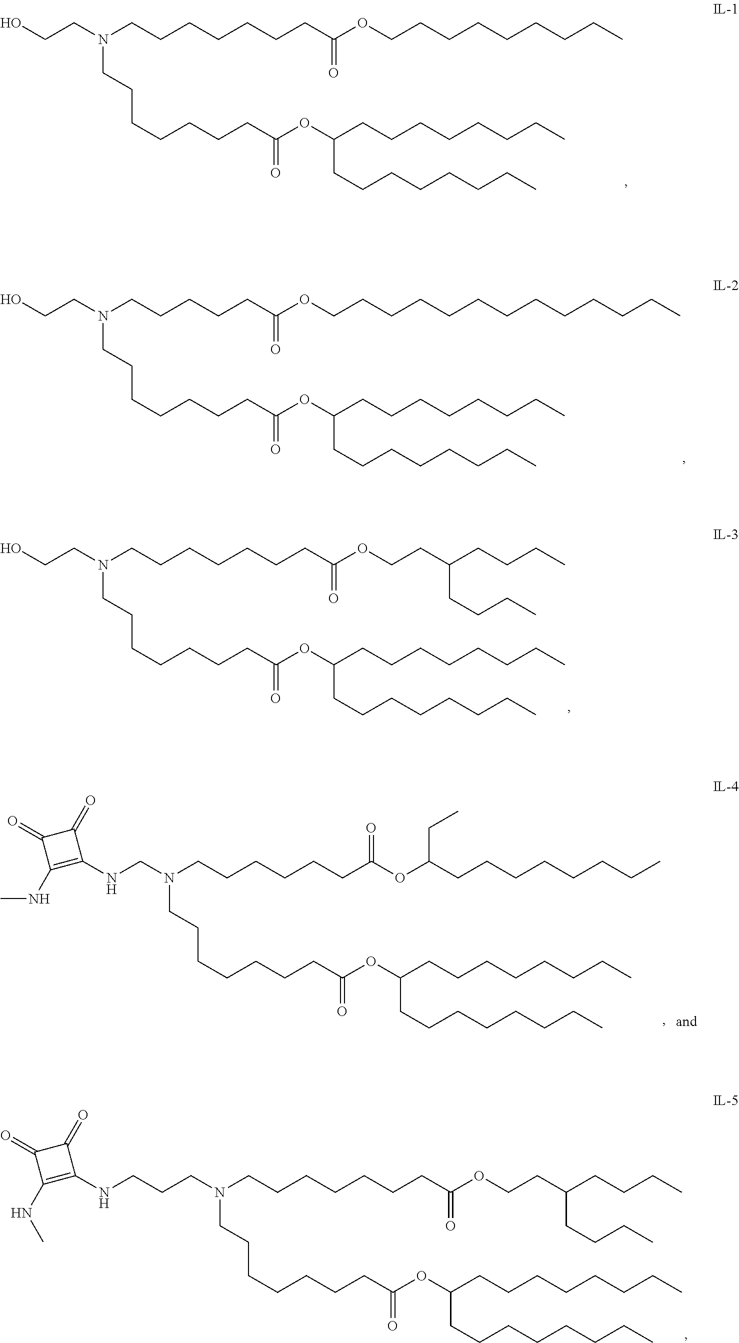
an N-oxide and/or salt of any of the foregoing, or a combination of any two or more thereof.

17. The TLA of claim 10, wherein the TLA comprises 30 to 65 mol % ionizable lipid.

18. The TLA of claim 10, wherein the structural lipid is selected from the group consisting of a sterol, a tocopherol, and mixtures of two or more thereof.

19. The TLA of claim 10, further comprising a therapeutic and/or prophylactic agent comprising a polyribonucleotide.

20. A method of specifically delivering a therapeutic and/or prophylactic agent to a target cell within a subject, producing a polypeptide of interest in a target cell within a subject, method of editing a gene in a target cell within a subject, the method comprising administering the TLA of claim 19 to the subject.
